(19) United States
(12) Patent Application Publication
(10) Pub. No.: US 2023/0203122 A1
(43) Pub. Date: Jun. 29, 2023

(54) POLYNUCLEOTIDES COMPRISING AN ANTIGENIC PAYLOAD

(71) Applicant: NUTCRACKER THERAPEUTICS, INC., Emeryville, CA (US)

(72) Inventors: Daniel Omar Frimannsson, Emeryville, CA (US); Ole Haabeth, Emeryville, CA (US)

(21) Appl. No.: 17/998,574

(22) PCT Filed: May 12, 2021
(86) PCT No.: PCT/US2021/031947

§ 371 Date: Nov. 11, 2022

Related U.S. Application Data
(60) Provisional application No. 63/024,604, filed on May 14, 2020.

(51) Int. Cl. C07K 14/705; A61K 39/39; A61K 45/06

(52) U.S. Cl. CPC C07K 14/705; A61K 39/39; A61K 45/06; A61K 2039/6018

(57) ABSTRACT

Polynucleotides, scaffolds, and cassettes are presently disclosed and described. In particular, these polynucleotides may have a formula comprising Signal/Leader-payload-PRM, wherein the Signal/Leader encodes a signal sequence, a leader sequence, or a sorting sequence, in frame with and upstream of a payload; the payload is an antigenic payload region, a detectable agent, and a therapeutic agent; and the PRM encodes all or a portion of at least one parental receptor molecule region from one or more isoforms or proteins selected from the group consisting of CD1d, CD1e, LDLR, LDLRP, and LRP1 proteins.

### BACKGROUND

Stimulation of both CD8+ and CD4+ lymphocytes is desirable for effective immunotherapy with recombinant vaccines, and in recent years, vaccines based on DNA or RNA nucleic acids have become increasingly important. However, these types of vaccines suffer from little or no stimulation of CD4+ lymphocytes, an element important for the efficacy of recombinant vaccines. Thus, a number of genetic manipulations have been developed to increase the immunogenicity of vaccines, e.g., by altering the primary sequence of fusion to foreign epitopes from bacteria or viruses and by chimeric products consisting of an antigen and immunomodulators such as cytokines or chemokines.

### SUMMARY

The present disclosure provides examples related to polynucleotides, scaffolds, and cassettes. The present disclosure also provides examples related to fusion molecules which comprise one or more polypeptide antigens such as tumor antigens, neoantigens, patient-specific antigens, shared antigens, and infectious agent antigens, engineered as a payload incorporated into a scaffold where such scaffold comprises one or more regions of a parental receptor molecule, e.g., signal sequence, extracellular region, transmembrane region and/or cytoplasmic region, for antigen presentation at the surface of a cell or at a specific cellular compartment. The present disclosure also provides examples related to polynucleotides and scaffolds that can be used for many applications, including inducing an immune or therapeutic response in an animal. Specifically, the polynucleotides and scaffolds of the disclosure are based on designs exploiting the CD1 and other cell receptors.

The present disclosure also provides alternative vaccine modalities, including scaffolds and cassettes incorporating antigenic payloads for use as vaccines.

The present disclosure further describes polynucleotides, e.g., DNA, RNA, or mRNA, encoding the scaffolds and cassettes, and methods of making and using them.

One aspect of the disclosure relates to a polynucleotide having the formula: Signal/Leader-payload-TMD-CYD wherein the Signal/Leader encodes a signal sequence, a leader sequence, or a sorting sequence, in frame with and upstream of a payload; the payload is selected from the group consisting of an antigenic payload region, a detectable agent, and a therapeutic agent; the TMD encodes a portion of a transmembrane region from one or more proteins or isoforms selected from the group consisting of CD1d, CD1e, LDLR, LDLRP, and LRP1 proteins; and the CYD encodes all or a portion of a cytoplasmic region from one or more proteins or isoforms selected from the group consisting of CD1d, CD1e, LDLR, LDLRP, and LRP1 proteins.

In some aspects, the payload is an antigenic payload region having the formula (An1)n-Xo-(An2)p comprising: a first encoded antigenic payload (An1), wherein n is an integer from 1 to 10; an encoded linker region (X), wherein o is an integer from 0 to 10; and a second encoded antigenic payload (An2), wherein p is an integer from 0 to 10.

In some aspects, the first encoded or second encoded antigenic payload encodes all or a portion of a tumor antigen or an infectious agent antigen.

In some aspects, the first encoded or second encoded antigenic payload comprises sequence SIINFEKL.

In an aspect, the payload is a detectable agent selected from the group consisting of organic small molecules, inorganic compounds, nanoparticles, enzymes or enzyme substrates, fluorescent materials, luminescent materials, bioluminescent materials, chemiluminescent materials, radioactive materials, contrast agents, gadolinium, iron oxides, monocrystalline iron oxide nanoparticles, ultrasmall superparamagnetic iron oxide, manganese chelates, barium sulfate, iodinated contrast media, microbubbles, and perfluorocarbons.

In one aspect, TMD and the CYD are derived from the same isoform or protein. In another aspect, the TMD and the CYD are derived from different isoforms or proteins.

In an aspect, the Signal/Leader encodes a signal sequence, a leader sequence, or a sorting sequence from the same isoform or protein as the TMD, the CYD, or both. In one aspect, the TMD encodes the sequence MGLIALAVLACLLFLLIVGFT. In another aspect, the CYD encodes the sequence SRFKRQTSYQGVL. In yet another aspect, the signal sequence encodes the sequence MGCLLFLLLWALLQAWGSA.

One aspect of the disclosure relates to a polynucleotide having the formula Signal/Leader-payload-PRM wherein the Signal/Leader encodes a signal sequence, a leader sequence, or a sorting sequence, in frame with and upstream of a payload; the payload is selected from the group consisting of an antigenic payload region, a detectable agent, and a therapeutic agent; and the PRM encodes all or a portion of at least one parental receptor molecule region from one or more proteins or isoforms selected from the group consisting of CD1d, CD1e, LDLR, LDLRP, and LRP1 proteins.

In an aspect, the parental receptor molecule is selected from the group consisting of an extracellular region, a transmembrane region, and a cytoplasmic region.

One aspect of the disclosure relates to a host cell comprising at least one of the disclosed polynucleotides.

One aspect of the disclosure relates to a pharmaceutical composition comprising at least one of the disclosed polynucleotides or a host cell. In an aspect, the pharmaceutical composition is in the form of a vaccine. In another aspect, the pharmaceutical composition further comprises one or more pharmaceutically acceptable excipients or one or more additional pharmaceutically active ingredients. In another aspect, the pharmaceutically acceptable excipients are selected from the group consisting of antiadherents, antioxidants, binders, coatings, compression aids, disintegrants, dyes, emollients, emulsifiers, fillers, film formers or coatings, flavors, fragrances, glidants, lubricants, preservatives, printing inks, sorbents, suspending or dispersing agents, sweeteners, and waters of hydration

One aspect of the disclosure relates to a therapeutic polynucleotide comprising at least one of the disclosed polynucleotides formulated with a delivery vehicle. In an aspect, the polynucleotide is encapsulated with the delivery vehicle. In another aspect, the delivery vehicle is selected from the group consisting of amphipathic molecules, amino-lipidated peptides, and tertiary amino lipidated cationic peptides.

One aspect of the disclosure relates to a therapeutic composition comprising a therapeutic polynucleotide. In another aspect, the therapeutic composition is in the form of a vaccine. In a further aspect, the therapeutic composition further comprises one or more therapeutically acceptable excipients or one or more additional therapeutically active ingredients. In an aspect, the therapeutically acceptable excipients are selected from the group consisting of antiadherents, antioxidants, binders, coatings, compression aids, disintegrants, dyes, emollients, emulsifiers, fillers, film formers or coatings, flavors, fragrances, glidants, lubricants, preservatives, printing inks, sorbents, suspending or dispersing agents, sweeteners, and waters of hydration.

An aspect of the disclosure includes administering at least one of the disclosed pharmaceutical compositions or therapeutic compositions, in particular, wherein a therapeutically effective dose, prophylactically effective dose, or appropriate imaging dose of the pharmaceutical composition or therapeutic composition is administered to a subject in need thereof.

One aspect of the disclosure includes methods of treating, vaccinating, or immunizing a subject in need thereof, the method comprising administering to the subject at least one of the disclosed polynucleotides, a host cell, at least one of the disclosed pharmaceutical compositions, or at least one of the disclosed therapeutic composition.

In an aspect, the subject is a mammal. In another aspect, the subject is a human.

In one aspect of the disclosure, the disclosed polynucleotides, including, but not limited to, the disclosed host cell, the disclosed pharmaceutical compositions, the disclosed therapeutic polynucleotides, the disclosed therapeutic compositions, or the disclosed methods, wherein the polynucleotide is to perform one of the following: a) enable antigen processing and presentation; b) traffic protein to the antigen presentation pathway; c) improve T cell activation; d) increase clonal diversity; and e) any combination thereof.

One aspect of the disclosure relates to a polynucleotide having the formula [Signal/Leader]—[(An1)n-Xo-(An2)p]—[TMD]—[CYD]] wherein [Signal/Leader] encodes any signal, leader, or sorting sequence in frame with, and upstream of, an antigenic payload region; [(An1)n-Xo-(An2)p] comprises an antigenic payload region, said antigenic payload region comprising (a) a first encoded antigenic payload (An1) which may be duplicated “n” number of times, (b) optionally, an encoded linker region (X) which may be duplicated “o” number of times, and (c) optionally, a second encoded antigenic payload (An2) which, when present, may be duplicated “p” number of times; TMD encodes a portion of a transmembrane region from one or more proteins selected from the group consisting of CD1, LDLR, LDLRP and/or LRP1 proteins; and CYD encodes all or portion of a cytoplasmic region from one or more proteins selected from the group consisting of CD1, LDLR, LDLRP and/or LRP1 proteins.

In an aspect, TMD and CYD are derived from a CD1 isoform. In another aspect, TMD and CYD are derived from the CD1d isoform. In an aspect, said TMD encodes the sequence MGLIALAVLACLLFLLIVGFT. In another aspect, said CYD encodes the sequence SRFKRQTSYQGVL. In yet another aspect, the signal sequence encodes the sequence MGCLLFLLLWALLQAWGSA. In another aspect, the encoded antigenic payload comprises the sequence SIINFEKL.

Other aspects and features of the present disclosure will become apparent to those ordinarily skilled in the art upon review of the following description of specific aspects of the disclosure in conjunction with the accompanying figures.

It should be appreciated that all combinations of the foregoing concepts and additional concepts discussed in greater detail below (provided such concepts are not mutually inconsistent) are contemplated as being part of the inventive subject matter disclosed herein and may be employed to achieve the benefits as described herein.

### BRIEF DESCRIPTION OF THE DRAWINGS

FIG. 1A depicts, in one example, flow cytometry results comparing antigen presentation in the JAWS dendritic cell model for an epitope in the context of different scaffolds; untreated and murine.

FIG. 1B depicts, in one example, flow cytometry results comparing antigen presentation in the JAWS dendritic cell model for an epitope in the context of different scaffolds; human CD1d and human CD1b.

FIGS. 2A and 2B depict, in one example, flow cytometry results of mRNA with hCD1d MHC trafficking signal enhances CD8 T cell re-activation in vitro.

FIG. 3A illustrates, in one example, a comparison of IFNg T cell responses observed with Sec-hCD1d MHC-sorting sequences over peptides, native pp65 mRNA, and pp65 mRNA Sec-MITD.

FIG. 3B depicts, in one example, flow cytometry results of activated CD8 T cells in samples treated with Sec-hCD1d pp65 mRNA nanoparticles compared to native pp65 mRNA, and pp65 mRNA Sec-MITD.

FIG. 4A depicts, in one example, the comparison of CD8 T cell growth in cultures treated with 1 μg/mL of non-coding mRNA-nanoparticles, 2 μg/mL of pp65 peptides, and 1 μg/mL of mRNA encoding pp65 with Sec-hCD1d.

FIG. 4B depicts, in one example, flow cytometry results of T2 target cells.

FIG. 4C depicts, in one example, flow cytometry results for untreated target cells, peptide induced CD8 T cells, and mRNA induced CD8 T cells.

FIG. 4D illustrates, in one example, a comparison of % PI positive target cells for peptide induced CD8 T cells, mRNA induced CD8 T cells, antigen loaded T2 target cells, and antigen negative T2 target cells.

FIG. 5 depicts, in one example, clonal diversity among CD8 T cells sorted from pp65 Sec-hCD1d mRNA nanoparticle treated PBMCs compared to pp65 peptides treated.

FIGS. 6A and 6B depict, in one example, the HPV16 E7 protein expression in HEK293.

### DETAILED DESCRIPTION

Compositions

Scaffolds

Scaffolds of the present disclosure are derived from one or more regions of one or more parental polypeptides, e.g., receptor molecule(s). Such parental molecules may include, but are not limited to, CD1, LDLR, LDLRP and/or LRP1 families of receptors or proteins.

In some aspects, the parental molecule is selected from the CD1 glycoprotein family of receptors. CD1 proteins are encoded in a locus on human chromosome 1. This region encodes five CD1 isoforms (CD1a-e). These proteins are expressed at the cell surface and function as antigen-presenting molecules, except for CD1e, which is only expressed intracellularly and is involved in processing and editing lipid for presentations by the other human CD1 isoforms. The CD1 isomers traffic around the cell by association with various chaperons such as calnexin, calreticulin, and even B2M. The newly-synthesized unoccupied CD1 isomer egress to the plasma membrane from the ER and Golgi, followed by internalization and entry into different compartments through tyrosine-based sorting motifs that permits their binding with adapter proteins complex 2 and 3, which facilitates entry into a variety of endosomal compartments (early endosomes, recycling endosomes, late endosomes) and lysosomes, ultimately undertaking a similar trafficking pathway to that of MHC I molecules. Furthermore, CD1 isomers traffic via these endosomal compartments to load antigen, and in many instances, CD1 and MHC I and MHC II molecules are detected within the same compartment.

Cassettes

Disclosed herein are constructs and scaffolds for pharmaceutical and therapeutic compositions. As provided herein, a pharmaceutical or therapeutic composition described herein may comprise a scaffold that may carry or convey a payload, such as an antigenic payload, a detectable agent, or a therapeutic agent. The combination of the scaffold and an antigenic payload is referred herein as a cassette. In the example where the composition is a vaccine composition, the composition comprises a scaffold that is to carry and convey an antigenic payload; and the combination of this scaffold and the payload is a vaccine cassette. As used herein, a “cassette” is a polynucleotide (or its encoded polypeptide) encoding a scaffold and an antigenic payload. In one aspect, a cassette, with the scaffod and antigentic payload thereof, may function as a vaccine. A vaccine may be referred to as a substance used to stimulate the production of antibodies and provide immunity against one or several diseases, prepared from the causative agent of a disease, its products, or a synthetic substitute. Cassettes may be configured for administration directly or to be encoded in one or more polynucleotides for expression in a cell and may be encoded in DNA, RNA, or mRNA for administration.

According to the present disclosure, a cassette may comprise the following formula:

5′UTR-Signal/Leader-(An1)n-Xo-(An2)p-TMD-CYD-3′ UTR-PolyA

where “UTRs” are the untranslated regions located at the 5′ and 3′ ends of an mRNA construct, and “PolyA” refers to the polyadenylation site of the mRNA;

Signal/Leader refers to a suitable signal sequence, leader sequence, sorting sequence, in frame with and upstream of the antigenic payload region;

(An1)n-Xo-(An2)p refers to any suitable antigenic payload region comprising a first antigenic payload (An1), a spacer or linker region (X), and a second antigenic payload (An2). In some examples, n is an integer greater than 1. For example, n can be 1, 2, 3, 4, 5, 6, 7, 8, 9, or 10. In some examples, n can be greater than 10. In some examples, o is an integer greater than 0. For example, o can be 0, 1, 2, 3, 4, 5, 6, 7, 8, 9, or 10. In some examples, o can be greater than 10. In some examples, p is an integer greater than 0. For example, p can be 0, 1, 2, 3, 4, 5, 6, 7, 8, 9, or 10. In some examples, p can be greater than 10;

TMD refers to all or a portion of a transmembrane region from one or more CD1 isoform, LDLR, LDLRP and/or LRP1 proteins; and

CYD refers to all or portion of a cytoplasmic region from one or more CD1 isoform, LDLR, LDLRP and/or LRP1 proteins.

In some aspects, a cassette may comprise the following formula:

5′UTR-Signal/Leader-payload-PRM-3′ UTR-PolyA

where “UTRs” are the untranslated regions located at the 5′ and 3′ ends of an mRNA construct, and “PolyA” refers to the polyadenylation site of the mRNA;

Signal/Leader refers to a suitable signal sequence, leader sequence, sorting sequence, in frame with and upstream of the antigenic payload region;

payload refers to an antigenic payload region, a detectable agent, or a therapeutic agent;

and

PRM refers to all or a portion of at least one parental receptor molecule region from one or more proteins selected from one or more CD1 isoform, LDLR, LDLRP, and LRP1 proteins. For example, the parental receptor molecule region could be independently selected from an extracellular region, a transmembrane region, or a cytoplasmic region, or any combination thereof.

In some aspects, the scaffolds or cassettes of the disclosure include one or more of the signal sequence and/or cytoplasmic sorting signal of CD1 isoform, LDLR, LDLRP and/or LRP1 isomers to facilitate antigen routing into the endosomal and/or lysosomal compartments, ultimately allowing the processing and loading of MHC Class I and MHC Class II molecules.

In some aspects, the signal sequence is selected from Human CD1a (MLFLLLPLLAVLPGDG); Human CD1b (MLLLPFQLLAVLFPGGN); Human CD1c (MLFLQFLLLALLLPGGD); Human CD1d (MGCLLFLLLWALLQAWGSA); Human CD1e (MLLLFLLFEGLCCPGENTA); Human LDLR (MGPWGWKLRWTVALLLAAAGT); or Human LRP1 (MLTPPLLLLLPLLSALVAA). According to the present disclosure, signal sequences may be derived from any protein. Signal sequences may range from 4-50 amino acids and may be chimeric, tandom, repeated, or inverted. Signal sequences may include those taught herein or any signal sequences that are at least about 50—e.g., at least about 60, about 70, about 80, about 90, about 95, about 99%, or higher, identical to those taught herein, as long as the signaling function is substantially retained.

In some aspects, the transmembrane domain sequence is selected from Human CD1a (GFIILAVIVPLLLLIGLALWF); Human CD1b (IVLAIIVPSLLLLLCLALWYM); Human CD1c (NWIALVVIVPLVILIVLVLWF); Human CD1d (MGLIALAVLACLLFLLIVGFT); Human CD1e (SIFLILICLTVIVTLVILVVV); Human LDLR (ALSIVLPIVLLVFLCLGVFLLW); or Human LRP1 (HIASILIPLLLLLLLVLVAGVVFWY). According to the present disclosure, transmembrane domain sequences may be derived from any protein. Transmembrane sequences may range from 10-100 amino acids and may be chimeric, tandom, repeated, or inverted. Transmembrane sequences may include those taught herein or any transmembrane sequences that are at least—e.g., at least about 60, about 70, about 80, about 90, about 95, about 99%, or higher, identical to those taught herein, as long as the function is substantially retained.

In some aspects, the cytoplasmic domain sequence is selected from Human CD1a (RKRCFC); Human CD1b (RRRSYQNIP); Human CD1c (KKHCSYQDIL); Human CD1d (SRFKRQTSYQGVL); Human CD1e (DSRLKKQSSNKNILSPHTPSPVFLMGANTQDTKNSRHQFCLAQVSWIKNRVLKKWKTR LNQLW); Human LDLR (KNWRLKNINSINFDNPVYQKTTEDEVHICHNQDGYSYPSRQMVSLEDDVA); and Human LRP1 (KRRVQGAKGFQHQRMTNGAMNVEIGNPTYKMYEGGEPDDVGGLLDADFALDPDKPT NFTNPVYATLYMGGHGSRHSLASTDEKRELLGRGPEDEIGDPLA). According to the present disclosure, cytoplasmic domain sequences may be derived from any protein. Cytoplasmic sequences may range from 10-100 amino acids and may be chimeric, tandom, repeated, or inverted. Cytoplasmic sequences may include those taught herein or any cytoplasmic sequences that are at least about 50— e.g., at least about 60, about 70, about 80, about 90, about 95, about 99%, or higher, identical to those taught herein, as long as the function is substantially retained.

It is noted that the CD1e sequence structure also contains an N-terminal propeptide sequence (APQALQSYHLAA) that is processed in endosomal compartments and is responsible for membrane association, while its absence results in a soluble molecule.

The NCBI reference for each of the above referenced parental receptor molecules is provided in Table 1.

TABLE 1
Reference Sequences

| PROTEIN ID | NCBI mRNA Reference Sequence |
|---|---|
| Human CD1a | NM_001320652.2 |
| Human CD1b | NM_001764.3 |
| Human CD1c | NM_001765.3 |
| Human CD1d | NM_001319145.2 |
| Human CD1e | NM_001042583.3 |
| Human LDLR | NM_000527.5 |
| Human LRP1 | NM_002332.3 |

Antigenic Payloads

The scaffolds of the present disclosure are engineered such that they may be loaded with or have incorporated therein at least one antigenic payload. Once an antigenic payload is combined with a scaffold, the construct is herein referred to as a cassette. In one aspect, the scaffold is a vaccine scaffold, and the construct therefore is referred to as a vaccine cassette.

Pharmaceutical and Therapeutic Compositions

Various diseases, disorders, and/or conditions may be treated with the pharmaceutical compositions. Pharmaceutical compositions may also comprise one or more pharmaceutically acceptable excipients or one or more additional pharmaceutically active ingredients.

Suitable non-limiting examples of pharmaceutically acceptable excipients include antiadherents, antioxidants, binders, coatings, compression aids, disintegrants, dyes, emollients, emulsifiers, fillers, film formers or coatings, flavors, fragrances, glidants, lubricants, preservatives, printing inks, sorbents, suspending or dispersing agents, sweeteners, and waters of hydration.

Pharmaceutically active ingredients include any substance or mixture thereof intended to provide pharmacological activity or other direct effects to diagnose, cure, mitigate, treat, or prevent a disease, disorder, and/or condition.

Therapeutic compositions may be used to treat a disease or to prevent a disease from happening, or to mitigate the symptoms of such a disease.

In some aspects, therapeutic compositions may comprise at least one polynucleotide of the present disclosure that is formulated or encapsulated by a delivery vehicle. This formulated or encapsulated polynucleotide is also referred to as a “therapeutic polynucleotide”. In some examples, the delivery vehicle is an amphipathic molecule, peptoid, amino-lipidated peptides, or tertiary amino lipidated cationic peptides. Therapeutic compositions may also comprise one or more therapeutically acceptable excipients or one or more additional therapeutically active ingredients.

Suitable non-limiting therapeutically acceptable excipients include antiadherents, antioxidants, binders, coatings, compression aids, disintegrants, dyes, emollients, emulsifiers, fillers, film formers or coatings, flavors, fragrances, glidants, lubricants, preservatives, printing inks, sorbents, suspending or dispersing agents, sweeteners, and waters of hydration.

Therapeutically active ingredients include any substance or mixture thereof intended to provide therapeutic activity or other direct effects to diagnose, cure, mitigate, treat, or prevent a disease, disorder, and/or condition.

Such diseases include cancer or infectious diseases. If cancer is the disease diagnosed, cured, mitigated, treated, or prevented with a pharmaceutical or therapeutic composition of the present disclosure, the antigenic payload may encode all or a portion of at least one tumor antigen. The tumor antigen may be a tumor-specific antigen (TSA) or a tumor-associated antigen (TAA). If an infectious disease is the disease diagnosed, cured, mitigated, treated, or prevented with the pharmaceutical or therapeutic composition of the present disclosure, the antigenic payload may encode all or a portion of at least one infectious agent antigen.

One example of such pharmaceutical compositions or therapeutic compositions is a vaccine. In an example of a vaccine of the present disclosure, the vaccine cassettes include one or more antigenic payload derived from a protein for which an immune response is desired.

As used herein, the term “cancer” refers to any of various malignant neoplasms characterized by the proliferation of anaplastic cells that tend to invade surrounding tissue and metastasize to new body sites and also refers to the pathological condition characterized by such malignant neoplastic growths. Cancers may be tumors or hematological malignancies, and include but are not limited to, all types of lymphomas/leukemias, carcinomas, and sarcomas, such as those cancers or tumors found in the anus, bladder, bile duct, bone, brain, breast, cervix, colon/rectum, endometrium, esophagus, eye, gallbladder, head and neck, liver, kidney, larynx, lung, mediastinum (chest), mouth, ovaries, pancreas, penis, prostate, skin, small intestine, stomach, spinal marrow, tailbone, testicles, thyroid, and uterus.

Types of carcinomas that may be treated with the pharmaceutical or therapeutic compositions present disclosure include, but are not limited to, soft tissue sarcoma such as alveolar soft part sarcoma, angiosarcoma, dermatofibrosarcoma, desmoid tumor, desmoplastic small round cell tumor, extraskeletal chondrosarcoma, extraskeletal osteosarcoma, fibrosarcoma, hemangiopericytoma, hemangiosarcoma, Kaposi's sarcoma, leiomyosarcoma, liposarcoma, lymphangiosarcoma, lymphosarcoma, malignant fibrous histiocytoma, neurofibrosarcoma, rhabdomyosarcoma, synovial sarcoma, and Askin's tumor, Ewing's sarcoma (primitive neuroectodermal tumor), malignant hemangioendothelioma, malignant schwannoma, osteosarcoma, and chondrosarcoma.

As a non-limiting example, the carcinoma which may be treated may be Acute granulocytic leukemia, Acute lymphocytic leukemia, Acute myelogenous leukemia, Adenocarcinoma, Adenosarcoma, Adrenal cancer, Adrenocortical carcinoma, Anal cancer, Anaplastic astrocytoma, Angiosarcoma, Appendix cancer, Astrocytoma, Basal cell carcinoma, B-Cell lymphoma), Bile duct cancer, Bladder cancer, Bone cancer, Bowel cancer, Brain cancer, Brain stem glioma, Brain tumor, Breast cancer, Carcinoid tumors, Cervical cancer, Cholangiocarcinoma, Chondrosarcoma, Chronic lymphocytic leukemia, Chronic myelogenous leukemia, Colon cancer, Colorectal cancer, Craniopharyngioma, Cutaneous lymphoma, Cutaneous melanoma, Diffuse astrocytoma, Ductal carcinoma in situ, Endometrial cancer, Ependymoma, Epithelioid sarcoma, Esophageal cancer, Ewing sarcoma, Extrahepatic bile duct cancer, Eye cancer, Fallopian tube cancer, Fibrosarcoma, Gallbladder cancer, Gastric cancer, Gastrointestinal cancer, Gastrointestinal carcinoid cancer, Gastrointestinal stromal tumors, General, Germ cell tumor, Glioblastoma multiforme, Glioma, Hairy cell leukemia, Head and neck cancer, Hemangioendothelioma, Hodgkin lymphoma, Hodgkin's disease, Hodgkin's lymphoma, Hypopharyngeal cancer, Infiltrating ductal carcinoma, Infiltrating lobular carcinoma, Inflammatory breast cancer, Intestinal Cancer, Intrahepatic bile duct cancer, Invasive/infiltrating breast cancer, Islet cell cancer, Jaw cancer, Kaposi sarcoma, Kidney cancer, Laryngeal cancer, Leiomyosarcoma, Leptomeningeal metastases, Leukemia, Lip cancer, Liposarcoma, Liver cancer, Lobular carcinoma in situ, Low-grade astrocytoma, Lung cancer, Lymph node cancer, Lymphoma, Male breast cancer, Medullary carcinoma, Medulloblastoma, Melanoma, Meningioma, Merkel cell carcinoma, Mesenchymal chondrosarcoma, Mesenchymous, Mesothelioma, Metastatic breast cancer, Metastatic melanoma, Metastatic squamous neck cancer, Mixed gliomas, Mouth cancer, Mucinous carcinoma, Mucosal melanoma, Multiple myeloma, Nasal cavity cancer, Nasopharyngeal cancer, Neck cancer, Neuroblastoma, Neuroendocrine tumors, Non-Hodgkin lymphoma, Non-Hodgkin's lymphoma, Non-small cell lung cancer, Oat cell cancer, Ocular cancer, Ocular melanoma, Oligodendroglioma, Oral cancer, Oral cavity cancer, Oropharyngeal cancer, Osteogenic sarcoma, Osteosarcoma, Ovarian cancer, Ovarian epithelial cancer, Ovarian germ cell tumor, Ovarian primary peritoneal carcinoma, Ovarian sex cord stromal tumor, Paget's disease, Pancreatic cancer, Papillary carcinoma, Paranasal sinus cancer, Parathyroid cancer, Pelvic cancer, Penile cancer, Peripheral nerve cancer, Peritoneal cancer, Pharyngeal cancer, Pheochromocytoma, Pilocytic astrocytoma, Pineal region tumor, Pineoblastoma, Pituitary gland cancer, Primary central nervous system lymphoma, Prostate cancer, Rectal cancer, Renal cell cancer, Renal pelvis cancer, Rhabdomyosarcoma, Salivary gland cancer, Sarcoma, Sarcoma, bone, Sarcoma, soft tissue, Sarcoma, uterine, Sinus cancer, Skin cancer, Small cell lung cancer, Small intestine cancer, Soft tissue sarcoma, Spinal cancer, Spinal column cancer, Spinal cord cancer, Spinal tumor, Squamous cell carcinoma, Stomach cancer, Synovial sarcoma, T-cell lymphoma), Testicular cancer, Throat cancer, Thymoma/thymic carcinoma, Thyroid cancer, Tongue cancer, Tonsil cancer, Transitional cell cancer, Transitional cell cancer, Transitional cell cancer, Triple-negative breast cancer, Tubal cancer, Tubular carcinoma, Ureteral cancer, Ureteral cancer, Urethral cancer, Uterine adenocarcinoma, Uterine cancer, Uterine sarcoma, Vaginal cancer, and Vulvar cancer.

Various infectious diseases may be treated with the pharmaceutical or therapeutic compositions of the present disclosure. In some examples, cassettes include one or more antigenic payloads derived from the infection agent or organism. As used herein, the term “infectious disease” refers to any disorders caused by organisms such as bacteria, viruses, fungi, or parasites. As a non-limiting example, the infectious disease and/or the causative agents include acute bacterial rhinosinusitis, 14-day measles, Acne, Acrodermatitis chronica atrophicans (ACA)-(late skin manifestation of latent Lyme disease), Acute hemorrhagic conjunctivitis, Acute hemorrhagic cystitis, Acute rhinosinusitis, Adult T-cell Leukemia-Lymphoma (ATLL), African Sleeping Sickness, AIDS (Acquired Immunodeficiency Sydrome), Alveolar hydatid, Amebiasis, Amebic meningoencephalitis, Anaplasmosis, Anthrax, Arboviral or parainfectious, Ascariasis —(Roundworm infections), Aseptic meningitis, Athlete's foot (Tinea pedis), Australian tick typhus, Avian Influenza, Babesiosis, Bacillary angiomatosis, Bacterial meningitis, Bacterial vaginosis, Balanitis, Balantidiasis, Bang's disease, Barmah Forest virus infection, Bartonellosis (Verruga peruana; Carrion's disease; Oroya fever), Bat Lyssavirus Infection, Bay sore (Chiclero's ulcer), Baylisascaris infection (Racoon roundworm infection), Beaver fever, Beef tapeworm, Bejel (endemic syphilis), Biphasic meningoencephalitis, Black Bane, Black death, Black piedra, Blackwater Fever, Blastomycosis, Blennorrhea of the newborn, Blepharitis, Boils, Bornholm disease (pleurodynia), Borrelia miyamotoi Disease, Botulism, Boutonneuse fever, Brazilian purpuric fever, Break Bone fever, Brill, Bronchiolitis, Bronchitis, Brucellosis (Bang's disease), Bubonic plague, Bullous impetigo, Burkholderia mallei (Glanders), Burkholderia pseudomallei (Melioidosis), Buruli ulcers (also Mycoburuli ulcers), Busse, Busse-Buschke disease (Cryptococcosis), California group encephalitis, Campylobacteriosis, Candidiasis, Canefield fever (Canicola fever; 7-day fever; Weil's disease; leptospirosis; canefield fever), Canicola fever, Capillariasis, Carate, Carbapenem-resistant Enterobacteriaceae (CRE), Carbuncle, Carrion's disease, Cat Scratch fever, Cave disease, Central Asian hemorrhagic fever, Central European tick, Cervical cancer, Chagas disease, Chancroid (Soft chancre), Chicago disease, Chickenpox (Varicella), Chiclero's ulcer, Chikungunya fever, Chlamydial infection, Cholera, Chromoblastomycosis, Ciguatera, Clap, Clonorchiasis (Liver fluke infection), Clostridium Difficile Infection, ClostriDium Perfringens (Epsilon Toxin), Coccidioidomycosis fungal infection (Valley fever; desert rheumatism), Coenurosis, Colorado tick fever, Condyloma accuminata, Condyloma accuminata (Warts), Condyloma lata, Congo fever, Congo hemorrhagic fever virus, Conjunctivitis, cowpox, Crabs, Crimean, Croup, Cryptococcosis, Cryptosporidiosis (Crypto), Cutaneous Larval Migrans, Cyclosporiasis, Cystic hydatid, Cysticercosis, Cystitis, Czechoslovak tick, D68 (EV-D68), Dacryocytitis, Dandy fever, Darling's Disease, Deer fly fever, Dengue fever (1, 2, 3 and 4), Desert rheumatism, Devil's grip, Diphasic milk fever, Diphtheria, Disseminated Intravascular Coagulation, Dog tapeworm, Donovanosis, Donovanosis (Granuloma inguinale), Dracontiasis, Dracunculosis, Duke's disease, Dum Dum Disease, Durand-Nicholas-Favre disease, Dwarf tapeworm, E. Coli infection (E. Coli), Eastern equine encephalitis, Ebola Hemorrhagic Fever (Ebola virus disease EVD), Ectothrix, Ehrlichiosis (Sennetsu fever), Encephalitis, Endemic Relapsing fever, Endemic syphilis, Endophthalmitis, Endothrix, Enterobiasis (Pinworm infection), Enterotoxin—B Poisoning (Staph Food Poisoning), Enterovirus Infection, Epidemic Keratoconjunctivitis, Epidemic Relapsing fever, Epidemic typhus, Epiglottitis, Erysipelis, Erysipeloid (Erysipelothricosis), Erythema chronicum migrans, Erythema infectiosum, Erythema marginatum, Erythema multiforme, Erythema nodosum, Erythema nodosum leprosum, Erythrasma, Espundia, Eumycotic mycetoma, European blastomycosis, Exanthem subitum (Sixth disease), Eyeworm, Far Eastern tick, Fascioliasis, Fievre boutonneuse (Tick typhus), Fifth Disease (erythema infectiosum), Filatow-Dukes' Disease (Scalded Skin Syndrome; Ritter's Disease), Fish tapeworm, Fitz-Hugh-Curtis syndrome—Perihepatitis, Flinders Island Spotted Fever, Flu (Influenza), Folliculitis, Four Corners Disease, Four Corners Disease (Human Pulmonary Syndrome (HPS)), Frambesia, Francis disease, Furunculosis, Gas gangrene, Gastroenteritis, Genital Herpes, Genital Warts, German measles, Gerstmann-Straussler-Scheinker (GSS), Giardiasis, Gilchrist's disease, Gingivitis, Gingivostomatitis, Glanders, Glandular fever (infectious mononucleosis), Gnathostomiasis, Gonococcal Infection (Gonorrhea), Gonorrhea, Granuloma inguinale (Donovanosis), Guinea Worm, Haemophilus Influenza disease, Hamburger disease, Hansen's disease—leprosy, Hantaan disease, Hantaan-Korean hemorrhagic fever, Hantavirus Pulmonary Syndrome, Hantavirus Pulmonary Syndrome (HPS), Hard chancre, Hard measles, Haverhill fever—Rat bite fever, Head and Body Lice, Heartland fever, Helicobacterosis, Hemolytic Uremic Syndrome (HUS), Hepatitis A, Hepatitis B, Hepatitis C, Hepatitis D, Hepatitis E, Herpangina, Herpes—genital, Herpes labialis, Herpes—neonatal, Hidradenitis, Histoplasmosis, Histoplasmosis infection (Histoplasmosis), His-Werner disease, HIV infection, Hookworm infections, Hordeola, Hordeola (Stye), HTLV, HTLV-associated myelopathy (HAM), Human granulocytic ehrlichiosis, Human monocytic ehrlichiosis, Human Papillomarivus (HPV), Human Pulmonary Syndrome, Hydatid cyst, Hydrophobia, Impetigo, Including congenital (German Measles), Inclusion conjunctivitis, Inclusion conjunctivitis—Swimming Pool conjunctivitis-Pannus, Infantile diarrhea, Infectious Mononucleosis, Infectious myocarditis, Infectious pericarditis, Influenza, Isosporiasis, Israeli spotted fever, Japanese Encephalitis, Jock itch, Jorge Lobo disease—lobomycosis, Jungle yellow fever, Junin Argentinian hemorrhagic fever, Kala Azar, Kaposi's sarcoma, Keloidal blastomycosis, Keratoconjunctivitis, Kuru, Kyasanur forest disease, LaCrosse encephalitis, Lassa hemorrhagic fever, Legionellosis (Legionnaires Disease), Legionnaire's pneumonia, Lemierre's Syndrome (Postanginal septicemia), Lemming fever, Leprosy, Leptospirosis (Nanukayami fever; Weil's disease), Listeriosis (Listeria), Liver fluke infection, Lobo's mycosis, Lockjaw, Loiasis, Louping Ill, Ludwig's angina, Lung fluke infection, Lung fluke infection (Paragonimiasis), Lyme disease, Lymphogranuloma venereum infection (LGV), Machupo Bolivian hemorrhagic fever, Madura foot, Mal del pinto, Malaria, Malignant pustule, Malta fever, Marburg hemorrhagic fever, Masters disease, Maternal Sepsis (Puerperal fever), Measles, Mediterannean spotted fever, Melioidosis (Whitmore's disease), Meningitis, Meningococcal Disease, MERS, Milker's nodule, Molluscum contagiosum, Moniliasis, monkeypox, Mononucleosis, Mononucleosis-like syndrome, Montezuma's Revenge, Morbilli, MRSA (methicillin-resistant Staphylococcus aureus) infection, Mucormycosis-Zygomycosis, Multiple Organ Dysfunction Syndrome or MODS, Multiple-system atrophy (MSA), Mumps, Murine typhus, Murray Valley Encephalitis (MVE), Mycoburuli ulcers, Mycoburuli ulcers-Buruli ulcers, Mycotic vulvovaginitis, Myositis, Nanukayami fever, Necrotizing fasciitis, Necrotizing fasciitis-Type 1, Necrotizing fasciitis-Type 2, Negishi, New world spotted fever, Nocardiosis, Nongonococcal urethritis, Non-Polio (Non-Polio Enterovirus), Norovirus infection, North American blastomycosis, North Asian tick typhus, Norwalk virus infection, Norwegian itch, O'Hara disease, Omsk hemorrhagic fever, Onchoceriasis, Onychomycosis, Opisthorchiasis, Opthalmia neonatorium, Oral hairy leukoplakia, Orf, Oriental Sore, Oriental Spotted Fever, Ornithosis (Parrot fever; Psittacosis), Oroya fever, Otitis externa, Otitis media, Pannus, Paracoccidioidomycosis, Paragonimiasis, Paralytic Shellfish Poisoning (Paralytic Shellfish Poisoning), Paronychia (Whitlow), Parotitis, PCP pneumonia, Pediculosis, Peliosis hepatica, Pelvic Inflammatory Disease, Pertussis (also called Whooping cough), Phaeohyphomycosis, Pharyngoconjunctival fever, Piedra (White Piedra), Piedra (Black Piedra), Pigbel, Pink eye conjunctivitis, Pinta, Pinworm infection, Pitted Keratolysis, Pityriasis versicolor (Tinea versicolor), Plague; Bubonic, Pleurodynia, Pneumococcal Disease, Pneumocystosis, Pneumonia, Pneumonic (Plague), Polio or Poliomyelitis, Polycystic hydatid, Pontiac fever, Pork tapeworm, Po sada-Wernicke disease, Postanginal septicemia, Powassan, Progressive multifocal leukencephalopathy, Progressive Rubella Panencephalitis, Prostatitis, Pseudomembranous colitis, Psittacosis, Puerperal fever, Pustular Rash diseases (Small pox), Pyelonephritis, Pylephlebitis, Q-Fever, Quinsy, Quintana fever (5-day fever), Rabbit fever, Rabies, Racoon roundworm infection, Rat bite fever, Rat tapeworm, Reiter Syndrome, Relapsing fever, Respiratory syncytial virus (RSV) infection, Rheumatic fever, Rhodotorulosis, Ricin Poisoning, Rickettsialpox, Rickettsiosis, Rift Valley Fever, Ringworm, Ritter's Disease, River Blindness, Rocky Mountain spotted fever, Rose Handler's disease (Sporotrichosis), Rose rash of infants, Roseola, Ross River fever, Rotavirus infection, Roundworm infections, Rubella, Rubeola, Russian spring, Salmonellosis gastroenteritis, San Joaquin Valley fever, Sao Paulo Encephalitis, Sao Paulo fever, SARS, Scabies Infestation (Scabies) (Norwegian itch), Scalded Skin Syndrome, Scarlet fever (Scarlatina), Schistosomiasis, Scombroid, Scrub typhus, Sennetsu fever, Sepsis (Septic shock), Severe Acute Respiratory Syndrome, Severe Acute Respiratory Syndrome (SARS), Shiga Toxigenic Escherichia coli (STEC/VTEC), Shigellosis gastroenteritis (Shigella), Shinbone fever, Shingles, Shipping fever, Siberian tick typhus, Sinusitis, Sixth disease, Slapped cheek disease, Sleeping sickness, Smallpox (Variola), Snail Fever, Soft chancre, Southern tick associated rash illness, Sparganosis, Spelunker's disease, Sporadic typhus, Sporotrichosis, Spotted fever, Spring, St. Louis encephalitis, Staphylococcal Food Poisoning, Staphylococcal Infection, Strep. throat, Streptococcal Disease, Streptococcal Toxic-Shock Syndrome, Strongyloiciasis, Stye, Subacute Sclerosing Panencephalitis, Subacute Sclerosing Panencephalitis (SSPE), Sudden Acute Respiratory Syndrome, Sudden Rash, Swimmer's ear, Swimmer's Itch, Swimming Pool conjunctivitis, Sylvatic yellow fever, Syphilis, Systemic Inflammatory Response Syndrome (SIRS), Tabes dorsalis (tertiary syphilis), Taeniasis, Taiga encephalitis, Tanner's disease, Tapeworm infections, Temporal lobe encephalitis, Temporal lobe encephalitis, tetani (Lock Jaw), Tetanus Infection, Threadworm infections, Thrush, Tick, Tick typhus, Tinea barbae, Tinea capitis, Tinea corporis, Tinea cruris, Tinea manuum, Tinea nigra, Tinea pedis, Tinea unguium, Tinea versicolor, Torulopsosis, Torulosis, Toxic Shock Syndrome, Toxoplasmosis, transmissible spongioform (CJD), Traveler's diarrhea, Trench fever 5, Trichinellosis, Trichomoniasis, Trichomycosis axillaris, Trichuriasis, Tropical Spastic Paraparesis (TSP), Trypanosomiasis, Tuberculosis (TB), Tuberculousis, Tularemia, Typhoid Fever, Typhus fever, Ulcus molle, Undulant fever, Urban yellow fever, Urethritis, Vaginitis, Vaginosis, Vancomycin Intermediate (VISA), Vancomycin Resistant (VRSA), Varicella, Venezuelan Equine encephalitis, Verruga peruana, Vibrio cholerae (Cholera), Vibriosis (Vibrio), Vincent's disease or Trench mouth, Viral conjunctivitis, Viral Meningitis, Viral meningoencephalitis, Viral rash, Visceral Larval Migrans, Vomito negro, Vulvovaginitis, Warts, Waterhouse, Weil's disease, West Nile Fever, Western equine encephalitis, Whipple's disease, Whipworm infection, White Piedra, Whitlow, Whitmore's disease, Winter diarrhea, Wolhynia fever, Wool sorters' disease, Yaws, Yellow Fever, Yersinosis, Yersinosis (Yersinia), Zahorsky's disease, Zika virus disease, Zoster, Zygomycosis, John Cunningham Virus (JCV), Human immunodeficiency virus (HIV), Influenza virus, Hepatitis B, Hepatitis C, Hepatitis D, Respiratory syncytial virus (RSV), Herpes simplex virus 1 and 2, Human Cytomegalovirus, Epstein-Barr virus, Varicella zoster virus, Coronaviruses, Poxviruses, Enterovirus 71, Rubella virus, Human papilloma virus, Streptococcus pneumoniae, Streptococcus viridans, Staphylococcus aureus (S. aureus), Methicillin-resistant Staphylococcus aureus (MRSA), Vancomycin-intermediate Staphylococcus aureus (VISA), Vancomycin-resistant Staphylococcus aureus (VRSA), Staphylococcus epidermidis (S. epidermidis), Clostridium Tetani, Bordetella pertussis, Bordetella paratussis, Mycobacterium, Francisella Tularensis, Toxoplasma gondii, Candida (C. albicans, C. glabrata, C. parapsilosis, C. tropicalis, C. krusei and C. lusitaniae) and/or any other infectious diseases, disorders or syndromes.

Various toxins may be used as a component or antigenic payload of the vaccines or cassettes of the disclosure. Non-limited examples of such antigenic payloads include Ricin, Bacillus anthracis, Shiga toxin and Shiga-like toxin, Botulinum toxins.

Various peptides or proteins from agents causing tropical diseases may be used as a component or antigenic payload of the vaccines or cassettes of the disclosure. Non-limiting examples of tropical diseases and/or the causative agent of such disease include Chikungunya fever, Dengue fever, Chagas disease, Rabies, Malaria, Ebola virus, Marburg virus, West Nile Virus, Yellow Fever, Japanese encephalitis virus, St. Louis encephalitis virus.

Various peptides or proteins from agents causing foodborne illnesses may be used as a component or antigenic payload of the vaccines or cassettes of the present disclosure. Non-limited examples of foodborne illnesses and/or the causative agent of such illnesses or gastroenteritis include Rotavirus, Norwalk virus (Norovirus), Campylobacter jejuni, Clostridium difficile, Entamoeba histolytica, Helicobacter pyroli, Enterotoxin B of Staphylococcus aureus, Hepatitis A virus (HAV), Hepatitis E, Listeria monocytogenes, Salmonella, Clostridium perfringens, and Salmonella.

Various peptides or proteins from agents causing infections may be used as a component or antigenic payload of the vaccines or vaccine cassettes of the disclosure. Non-limited examples of such infectious agents include adenoviruses, Anaplasma phagocytophilium, Ascaris lumbricoides, Bacillus anthracis, Bacillus cereus, Bacteriodes sp, Barmah Forest virus, Bartonella bacilliformis, Bartonella henselae, Bartonella quintana, beta-toxin of Clostridium perfringens, Bordetella pertussis, Bordetella parapertussis, Borrelia burgdorferi, Borrelia miyamotoi, Borrelia recurrentis, Borrelia sp., Botulinum toxin, Brucella sp., Burkholderia pseudomallei, California encephalitis virus, Campylobacter, Candida albicans, chikungunya virus, Chlamydia psittaci, Chlamydia trachomatis, Clonorchis sinensis, Clostridium difficile bacteria, Clostridium tetani, Colorado tick fever virus, Corynebacterium diphtheriae, Corynebacterium minutissimum, Coxiella burnetii, coxsackie A, coxsackie B, Crimean-Congo hemorrhagic fever virus, cytomegalovirus, dengue virus, Eastern Equine encephalitis virus, Ebola viruses, echovirus, Ehrlichia chaffeensis, Ehrlichia equi, Ehrlichia sp., Entamoeba histolytica, Enterobacter sp., Enterococcus feacalis, Enterovirus 71, Epstein-Barr virus (EBV), Erysipelothrix rhusiopathiae, Escherichia coli, Flavivirus, Fusobacterium necrophorum, Gardnerella vaginalis, Group B streptococcus, Haemophilus aegyptius, Haemophilus ducreyi, Haemophilus influenzae, hantavirus, Helicobacter pylori, Hepatitis A, Hepatitis B, Hepatitis C, Hepatitis D, Hepatitis E, herpes simplex virus 1 and 2, human herpes virus 6, human herpes Virus 8, human immunodeficiency virus 1 and 2, human T-cell leukemia viruses I and II, influenza viruses (A, B, C), Jamestown Canyon virus, Japanese encephalitis antigenic, Japanese encephalitis virus, John Cunninham virus, juninvirus, Kaposi's Sarcoma-associated Herpes Virus (KSHV), Klebsiella granulomatis, Klebsiella sp., Kyasanur Forest Disease virus, La Crosse virus, Lassavirus, Legionella pneumophila, Leptospira interrogans, Listeria monocytogenes, lymphocytic choriomeningitis virus, lyssavirus, Machupovirus, Marburg virus, measles virus, MERS coronavirus (MERS-CoV), Micrococcus sedentarius, Mobiluncus sp., Molluscipoxvirus, Moraxella catarrhalis, Morbilli-Rubeola virus, Mumpsvirus, Mycobacterium leprae, Mycobacterium tuberculosis, Mycobacterium ulcerans, Mycoplasma genitalium, Mycoplasma sp, Nairovirus, Neisseria gonorrhoeae, Neisseria meningitidis, Nocardia, Norwalk virus, norovirus, Omsk hemorrhagic fever virus, papilloma virus, parainfluenza viruses 1-3, parapoxvirus, parvovirus B19, Peptostreptococccus sp., Plasmodium sp., polioviruses types I, II, and III, Proteus sp., Pseudomonas aeruginosa, Pseudomonas pseudomallei, Pseudomonas sp., rabies virus, respiratory syncytial virus, ricin toxin, Rickettsia australis, Rickettsia conori, Rickettsia honei, Rickettsia prowazekii, Ross River Virus, rotavirus, rubellavirus, Saint Louis encephalitis, Salmonella Typhi, Sarcoptes scabiei, SARS-associated coronavirus (SARS-CoV), SARS-associated coronavirus (SARS-CoV-2)Serratia sp., Shiga toxin and Shiga-like toxin, Shigella sp., Sin Nombre Virus, Snowshoe hare virus, Staphylococcus aureus, Staphylococcus epidermidis, Streptobacillus moniliformis, Streptoccoccus pneumoniae, Streptococcus agalactiae, Streptococcus agalactiae, Streptococcus group A-H, Streptococcus pneumoniae, Streptococcus pyogenes, Treponema pallidum subsp. Pallidum, Treponema pallidum var. carateum, Treponema pallidum var. endemicum, Tropheryma whippelii, Ureaplasma urealyticum, Varicella-Zoster virus, variola virus, Vibrio cholerae, West Nile virus, yellow fever virus, Yersinia enterocolitica, Yersinia pestis, and Zika virus.

Pharmaceutical and Therapeutic Compositions: Dosing, Administration, and Delivery

Dosing

The present disclosure provides methods comprising administering any one or more pharmaceutical or therapeutic compositions described herein to a subject in need thereof. The pharmaceutical or therapeutic composition may be, for example, a vaccine. These may be administered to a subject using any amount and any route of administration effective for preventing or treating, or imaging a disease, disorder, and/or condition (e.g., a disease, disorder, and/or condition). The exact amount required will vary from subject to subject, depending on the species, age, and general condition of the subject, the severity of the disease, the particular composition, its mode of administration, its mode of activity, and the like.

Compositions described herein may be formulated in dosage unit form for ease of administration and uniformity of dosage. It will be understood, however, that the total daily usage of the compositions described herein may be decided by the attending physician within the scope of sound medical judgment. The specific therapeutically effective, prophylactically effective, or appropriate imaging dose level for any particular patient will depend upon a variety of factors, including the disorder being treated and the severity of the disorder; the activity of the specific compound employed; the specific composition employed; the age, body weight, general health, sex and diet of the patient; the time of administration, route of administration, and rate of excretion of the specific compound employed; the duration of the treatment; drugs used in combination or coincidental with the specific compound employed; and like factors well known in the medical arts.

Administration

The pharmaceutical or therapeutic compositions of the present disclosure may be administered by any route to achieve a therapeutically effective outcome. These include, but are not limited to enteral (into the intestine), gastroenteral, epidural (into the dura matter), oral (by way of the mouth), transdermal, peridural, intracerebral (into the cerebrum), intracerebroventricular (into the cerebral ventricles), epicutaneous (application onto the skin), intradermal, (into the skin itself), subcutaneous (under the skin), nasal administration (through the nose), intravenous (into a vein), intravenous bolus, intravenous drip, intraarterial (into an artery), intramuscular (into a muscle), intracardiac (into the heart), intraosseous infusion (into the bone marrow), intrathecal (into the spinal canal), intraperitoneal, (infusion or injection into the peritoneum), intravesical infusion, intravitreal, (through the eye), intracavernous injection (into a pathologic cavity) intracavitary (into the base of the penis), intravaginal administration, intrauterine, extra-amniotic administration, transdermal (diffusion through the intact skin for systemic distribution), transmucosal (diffusion through a mucous membrane), transvaginal, insufflation (snorting), sublingual, sublabial, enema, eye drops (onto the conjunctiva), in ear drops, auricular (in or by way of the ear), buccal (directed toward the cheek), conjunctival, cutaneous, dental (to a tooth or teeth), electro-osmosis, endocervical, endosinusial, endotracheal, extracorporeal, hemodialysis, infiltration, interstitial, intra-abdominal, intra-amniotic, intra-articular, intrabiliary, intrabronchial, intrabursal, intracartilaginous (within a cartilage), intracaudal (within the cauda equine), intracisternal (within the cisterna magna cerebellomedularis), intracorneal (within the cornea), dental intracornal, intracoronary (within the coronary arteries), intracorporus cavernosum (within the dilatable spaces of the corporus cavernosa of the penis), intradiscal (within a disc), intraductal (within a duct of a gland), intraduodenal (within the duodenum), intradural (within or beneath the dura), intraepidermal (to the epidermis), intraesophageal (to the esophagus), intragastric (within the stomach), intragingival (within the gingivae), intraileal (within the distal portion of the small intestine), intralesional (within or introduced directly to a localized lesion), intraluminal (within a lumen of a tube), intralymphatic (within the lymph), intramedullary (within the marrow cavity of a bone), intrameningeal (within the meninges), intramyocardial (within the myocardium), intraocular (within the eye), intraovarian (within the ovary), intrapericardial (within the pericardium), intrapleural (within the pleura), intraprostatic (within the prostate gland), intrapulmonary (within the lungs or its bronchi), intrasinal (within the nasal or periorbital sinuses), intraspinal (within the vertebral column), intrasynovial (within the synovial cavity of a joint), intratendinous (within a tendon), intratesticular (within the testicle), intrathecal (within the cerebrospinal fluid at any level of the cerebrospinal axis), intrathoracic (within the thorax), intratubular (within the tubules of an organ), intratumor (within a tumor), intratympanic (within the aurus media), intravascular (within a vessel or vessels), intraventricular (within a ventricle), iontophoresis (by means of electric current where ions of soluble salts migrate into the tissues of the body), irrigation (to bathe or flush open wounds or body cavities), laryngeal (directly upon the larynx), nasogastric (through the nose and into the stomach), occlusive dressing technique (topical route administration which is then covered by a dressing which occludes the area), ophthalmic (to the external eye), oropharyngeal (directly to the mouth and pharynx), parenteral, percutaneous, periarticular, peridural, perineural, periodontal, rectal, respiratory (within the respiratory tract by inhaling orally or nasally for local or systemic effect), retrobulbar (behind the pons or behind the eyeball), intramyocardial (entering the myocardium), soft tissue, subarachnoid, subconjunctival, submucosal, topical, transplacental (through or across the placenta), transtracheal (through the wall of the trachea), transtympanic (across or through the tympanic cavity), ureteral (to the ureter), urethral (to the urethra), vaginal, caudal block, diagnostic, nerve block, biliary perfusion, cardiac perfusion, photopheresis or spinal.

Parenteral and Injectible Administration

In some aspects, pharmaceutical or therapeutic compositions of the present disclosure may be administered parenterally. Liquid dosage forms for oral and parenteral administration include, but are not limited to, pharmaceutically or therapeutically acceptable emulsions, microemulsions, solutions, suspensions, syrups, and/or elixirs. In addition to active ingredients, liquid dosage forms may comprise inert diluents commonly used in the art such as, for example, water or other solvents, solubilizing agents and emulsifiers such as ethyl alcohol, isopropyl alcohol, ethyl carbonate, ethyl acetate, benzyl alcohol, benzyl benzoate, propylene glycol, 1,3-butylene glycol, dimethylformamide, oils (in particular, cottonseed, groundnut, corn, germ, olive, castor, and sesame oils), glycerol, tetrahydrofurfuryl alcohol, polyethylene glycols and fatty acid esters of sorbitan, and mixtures thereof. Besides inert diluents, oral compositions can include adjuvants such as wetting agents, emulsifying and suspending agents, sweetening, flavoring, and/or perfuming agents. In certain aspects of parenteral administration, compositions are mixed with solubilizing agents such as CREMOPHOR®, alcohols, oils, modified oils, glycols, polysorbates, cyclodextrins, polymers, and/or combinations thereof. In other aspects, surfactants are included, such as hydroxypropylcellulose.

Injectable preparations, for example, sterile injectable aqueous or oleaginous suspensions, may be formulated according to the known art using suitable dispersing agents, wetting agents, and/or suspending agents. Sterile injectable preparations may be sterile injectable solutions, suspensions, and/or emulsions in nontoxic parenterally acceptable diluents and/or solvents, for example, as a solution in 1,3-butanediol. Among the acceptable vehicles and solvents that may be employed are water, Ringer's solution, U.S.P., and isotonic sodium chloride solution. Sterile, fixed oils are conventionally employed as a solvent or suspending medium. For this purpose, any bland fixed oil can be employed, including synthetic mono- or diglycerides. Fatty acids such as oleic acid can be used in the preparation of injectables.

Injectable formulations may be sterilized, for example, by filtration through a bacterial-retaining filter and/or by incorporating sterilizing agents in the form of sterile solid compositions, which can be dissolved or dispersed in sterile water or other sterile injectable media prior to use.

In order to prolong the effect of active ingredients, it is often desirable to slow the absorption of active ingredients from subcutaneous or intramuscular injections. This may be accomplished by the use of liquid suspensions of crystalline or amorphous material with poor water solubility. The rate of absorption of active ingredients depends upon the rate of dissolution, which, in turn, may depend upon crystal size and crystalline form. Alternatively, delayed absorption of a parenterally administered drug form is accomplished by dissolving or suspending the drug in an oil vehicle. Injectable depot forms are made by forming microencapsulated matrices of the drug in biodegradable polymers such as polylactide-polyglycolide. Depending upon the ratio of drug to polymer and the nature of the particular polymer employed, the rate of drug release can be controlled. Examples of other biodegradable polymers include poly(orthoesters) and poly(anhydrides). Depot injectable formulations are prepared by entrapping the drug in liposomes or microemulsions that are compatible with body tissues.

Rectal and Vaginal Administration

In some aspects, pharmaceutical or therapeutic compositions of the present disclosure may be administered rectally and/or vaginally. Compositions for rectal or vaginal administration are typically suppositories that can be prepared by mixing compositions with suitable non-irritating excipients such as cocoa butter, polyethylene glycol, or a suppository wax which are solid at ambient temperature but liquid at body temperature and therefore melt in the rectum or vaginal cavity and release the active ingredient.

Oral Administration

In some aspects, pharmaceutical or therapeutic compositions of the present disclosure may be administered orally. Solid dosage forms for oral administration include capsules, tablets, pills, powders, and granules. In such solid dosage forms, an active ingredient is mixed with at least one inert, pharmaceutically acceptable excipient such as sodium citrate or dicalcium phosphate and/or fillers or extenders (e.g., starches, lactose, sucrose, glucose, mannitol, and silicic acid), binders (e.g., carboxymethylcellulose, alginates, gelatin, polyvinylpyrrolidinone, sucrose, and acacia), humectants (e.g., glycerol), disintegrating agents (e.g., agar, calcium carbonate, potato or tapioca starch, alginic acid, certain silicates, and sodium carbonate), solution retarding agents (e.g., paraffin), absorption accelerators (e.g., quaternary ammonium compounds), wetting agents (e.g., cetyl alcohol and glycerol monostearate), absorbents (e.g., kaolin and bentonite clay), lubricants (e.g., talc, calcium stearate, magnesium stearate, solid polyethylene glycols, sodium lauryl sulfate), and mixtures thereof. In the case of capsules, tablets, and pills, the dosage form may comprise buffering agents.

Topical or Transdermal Administration

As described herein, pharmaceutical or therapeutic compositions of the present disclosure may be formulated for administration topically. The skin may be an ideal target site for delivery as it is readily accessible. Three routes are commonly considered to deliver pharmaceutical or therapeutic compositions of the present disclosure to the skin: (i) topical application (e.g., for local/regional treatment and/or cosmetic applications); (ii) intradermal injection (e.g., for local/regional treatment and/or cosmetic applications); and (iii) systemic delivery (e.g., for treatment of dermatologic diseases that affect both cutaneous and extracutaneous regions). Pharmaceutical compositions of the present disclosure can be delivered to the skin by several different approaches known in the art.

In some aspects, the disclosure provides for a variety of dressings (e.g., wound dressings) or bandages (e.g., adhesive bandages) for conveniently and/or effectively carrying out methods of the present disclosure. Typically dressing or bandages may comprise sufficient amounts of pharmaceutical or therapeutic compositions of the present disclosure described herein to allow users to perform multiple treatments.

Dosage forms for topical and/or transdermal administration may include ointments, pastes, creams, lotions, gels, powders, solutions, sprays, inhalants and/or patches. Generally, active ingredients are admixed under sterile conditions with pharmaceutically acceptable excipients and/or any needed preservatives and/or buffers. Additionally, the present disclosure contemplates the use of transdermal patches, which often have the added advantage of providing controlled delivery of pharmaceutical or therapeutic compositions of the present disclosure to the body. Such dosage forms may be prepared, for example, by dissolving and/or dispensing pharmaceutical or therapeutic compositions in the proper medium. Alternatively, or additionally, rates may be controlled by either providing rate controlling membranes and/or by dispersing pharmaceutical or therapeutic compositions in a polymer matrix and/or gel.

Formulations suitable for topical administration include, but are not limited to, liquid and/or semi liquid preparations such as liniments, lotions, oil in water and/or water in oil emulsions such as creams, ointments and/or pastes, and/or solutions and/or suspensions.

Topically administrable formulations may, for example, comprise from about 1% to about 10% (w/w) active ingredient, although the concentration of active ingredient may be as high as the solubility limit of the active ingredient in the solvent. Formulations for topical administration may further comprise one or more of the additional ingredients described herein.

Depot Administration

As described herein, in some aspects, pharmaceutical or therapeutic compositions of the present disclosure are formulated in depots for extended-release. Generally, specific organs or tissues (“target tissues”) are targeted for administration.

In some aspects of the disclosure, pharmaceutical or therapeutic compositions of the present disclosure are spatially retained within or proximal to target tissues. Disclosed are methods of providing pharmaceutical or therapeutic compositions to target tissues of mammalian subjects by contacting target tissues (which comprise one or more target cells) with pharmaceutical or therapeutic compositions under conditions such that they are substantially retained in target tissues, meaning that at least about 10—e.g., at least about 20, about 30, about 40, about 50, about 60, about 70, about 80, about 85, about 90, about 95, about 96, about 97, about 98, about 99, about 99.9, about 99.99, or greater, of the composition is retained in the target tissues. Advantageously, retention is determined by measuring the amount of pharmaceutical or therapeutic compositions that enter one or more target cells. For example, at least about 1%—e.g., about 5%, about 10%, about 20%, about 30%, about 40%, about 50%, about 60%, about 70%, about 80%, about 85%, about 90%, about 95%, about 96%, about 97%, about 98%, about 99%, about 99.9%, about 99.99% or greater than about 99.99% of pharmaceutical or therapeutic compositions administered to subjects are present intracellularly at a period of time following administration. For example, intramuscular injection to mammalian subjects may be performed using aqueous compositions comprising pharmaceutical or therapeutic compositions of the present disclosure and one or more transfection reagent, and retention is determined by measuring the amount of pharmaceutical or therapeutic compositions present in muscle cells.

Certain aspects of the disclosure are directed to methods of providing pharmaceutical or therapeutic compositions of the present disclosure to a target tissues of mammalian subjects, by contacting target tissues (comprising one or more target cells) with pharmaceutical or therapeutic compositions under conditions such that they are substantially retained in such target tissues. Pharmaceutical or therapeutic compositions comprise enough active ingredient(s) such that the effect of interest is produced in at least one target cell. In some aspects, pharmaceutical or therapeutic compositions generally comprise one or more cell penetration agents, although “naked” formulations (such as without cell penetration agents or other agents) are also contemplated, with or without pharmaceutically or therapeutically acceptable carriers.

In some aspects, formulations comprise a plurality of different pharmaceutical or therapeutic compositions. Optionally, formulations may also comprise cell penetration agents to assist in the intracellular delivery of pharmaceutical or therapeutic compositions. In such aspects, determinations are made of compound and/or composition dose required to target biomolecules of interest in substantial percentages of cells contained within predetermined volumes of the target tissue (generally, without targeting biomolecules of interest in adjacent or distal tissues.) Determined doses are then introduced directly into subject tissues.

Pulmonary Administration

In some aspects, pharmaceutical or therapeutic compositions of the present disclosure may be prepared, packaged, and/or sold in formulations suitable for pulmonary administration. In some aspects, such administration is via the buccal cavity. In some aspects, formulations may comprise dry particles comprising active ingredients. In such aspects, dry particles may have a diameter in the range from about 0.5 nm to about 7 nm or from about 1 nm to about 6 nm. In some aspects, formulations may be in the form of dry powders for administration using devices comprising dry powder reservoirs to which streams of propellant may be directed to disperse such powder. In some aspects, self-propelling solvent/powder dispensing containers may be used. In such aspects, active ingredients may be dissolved and/or suspended in low-boiling propellant in sealed containers. Such powders may comprise particles wherein at least 98% of the particles by weight have diameters greater than 0.5 nm and at least 95% of the particles by number have diameters less than about 7 nm. Alternatively, at least about 95% of the particles by weight have a diameter greater than about 1 nm and at least about 90% of the particles by number have a diameter less than about 6 nm. Dry powder compositions may include a solid fine powder diluent such as sugar and are conveniently provided in a unit dose form.

Low boiling propellants generally include liquid propellants having a boiling point of below 65° F. at atmospheric pressure. Generally, propellants may constitute about 50% to about 99.9% (w/w) of the composition, and active ingredient(s) may constitute about 0.1% to about 20% (w/w) of the composition. Propellants may further comprise additional ingredients such as liquid non-ionic and/or solid anionic surfactant and/or a solid diluent (which may have particle sizes of the same order as particles comprising active ingredients).

Pharmaceutical or therapeutic compositions formulated for pulmonary delivery may provide active ingredients in the form of droplets of a solution and/or suspension. Such formulations may be prepared, packaged, and/or sold as aqueous and/or dilute alcoholic solutions and/or suspensions, optionally sterile, comprising active ingredients, and may conveniently be administered using any nebulization and/or atomization device. Such formulations may further comprise one or more additional ingredients including, but not limited to, a flavoring agent such as saccharin sodium, a volatile oil, a buffering agent, a surface active agent, and/or a preservative such as methylhydroxybenzoate.

Intranasal, Nasal, and Buccal Administration

In some aspects, pharmaceutical or therapeutic compositions of the present disclosure may be administered nasally and/or intranasally. In some aspects, formulations described herein as being useful for pulmonary delivery may also be useful for intranasal delivery. In some aspects, formulations for intranasal administration comprise a coarse powder comprising the active ingredient and having an average particle from about 0.2 um to about 500 um. Such formulations are administered in the manner in which snuff is taken, i.e., by rapid inhalation through the nasal passage from a container of the powder held close to the nose.

Formulations suitable for nasal administration may, for example, comprise from about as little as about 0.1% (w/w) and as much as about 100% (w/w) of active ingredient(s), and may comprise one or more of the additional ingredients described herein. A pharmaceutical or therapeutic composition may be prepared, packaged, and/or sold in a formulation suitable for buccal administration. Such formulations may, for example, be in the form of tablets and/or lozenges made using conventional methods, and may, for example, about 0.1% to about 20% (w/w) active ingredient, the balance comprising an orally dissolvable and/or degradable composition and, optionally, one or more of the additional ingredients described herein. Alternately, formulations suitable for buccal administration may comprise powders and/or aerosolized and/or atomized solutions and/or suspensions comprising active ingredients. Such powdered, aerosolized, and/or aerosolized formulations, when dispersed, may comprise average particle and/or droplet sizes in the range of from about 0.1 nm to about 200 nm, and may further comprise one or more of any additional ingredients described herein.

Ophthalmic or Otic Administration

In some aspects, pharmaceutical or therapeutic compositions of the present disclosure may be prepared, packaged, and/or sold in formulations suitable for ophthalmic and/or otic administration. Such formulations may, for example, be in the form of eye and/or ear drops including, for example, a 0.1/1.0% (w/w) solution and/or suspension of the active ingredient in aqueous and/or oily liquid excipients. Such drops may further comprise buffering agents, salts, and/or one or more other of any additional ingredients described herein. Other ophthalmically-administrable formulations that are useful include those which comprise active ingredients in microcrystalline form and/or in liposomal preparations. Subretinal inserts may also be used as forms of administration.

Delivery Vehicle Molecule

Delivery Modalities

The pharmaceutical or therapeutic compositions may be delivered using one or more modalities. These include viral vectors and particles such as lentivirus, adenovirus, adeno-associated virus, herpes simplex virus, retrovirus, and the like. Other modalities may also be used such as mRNA, plasmids, and recombinant proteins.

Lentiviral Vehicles

In some aspects, lentiviral vehicles/particles may be used as delivery modalities. Lentiviruses are a subgroup of the Retroviridae family of viruses, named because reverse transcription of viral RNA genomes to DNA is required before integration into the host genome. As such, the most important features of lentiviral vehicles/particles are the integration of their genetic material into the genome of a target/host cell. Some examples of lentivirus include the Human Immunodeficiency Viruses: HIV-1 and HIV-2, the Simian Immunodeficiency Virus (SIV), feline immunodeficiency virus (FIV), bovine immunodeficiency virus (BIV), Jembrana Disease Virus (JDV), equine infectious anemia virus (EIAV), equine infectious anemia virus, visna-maedi and caprine arthritis encephalitis virus (CAEV).

Lentiviral particles making up the gene delivery vehicle may be replication defective on their own (also referred to as “self-inactivating”). Lentiviruses can infect both dividing and non-dividing cells by virtue of the entry mechanism through the intact host nuclear envelope (Naldini L et al., Curr. Opin. Biotechnol, 1998, 9: 457-463). Recombinant lentiviral vehicles/particles have been generated by multiply attenuating the HIV virulence genes, for example, the genes Env, Vif, Vpr, Vpu, Nef, and Tat are deleted making the vector biologically safe. Correspondingly, lentiviral vehicles, for example, derived from HIV-1/HIV-2 can mediate the efficient delivery, integration, and long-term expression of transgenes into non-dividing cells.

Lentiviral particles may be generated by co-expressing the virus packaging elements and the vector genome itself in a producer cell such as human HEK293T cells. These elements are usually provided in three or four separate plasmids. The producer cells are co-transfected with plasmids that encode lentiviral components, including the core (i.e., structural proteins) and enzymatic components of the virus, and the envelope protein(s) (referred to as the packaging systems), and a plasmid that encodes the genome including a foreign transgene, to be transferred to the target cell, the vehicle itself (also referred to as the transfer vector). In general, the plasmids or vectors are included in a producer cell line. The plasmids/vectors are introduced via transfection, transduction, or infection into the producer cell line. Methods for transfection, transduction, or infection are well known by those of skill in the art. As a non-limiting example, the packaging and transfer constructs can be introduced into producer cell lines by calcium phosphate transfection, lipofection, or electroporation, generally together with a dominant selectable marker, such as neo, DHFR, Gln synthetase, or ADA, followed by selection in the presence of the appropriate drug and isolation of clones.

The producer cell produces recombinant viral particles that contain the foreign gene, for example, the vaccine or vaccine cassette of the present disclosure. The recombinant viral particles are recovered from the culture media and titrated by standard methods used by those of skill in the art. The recombinant lentiviral vehicles can be used to infect target cells.

Cells that can be used to produce high-titer lentiviral particles may include, but are not limited to, HEK293T cells, 293G cells, STAR cells (Relander et al., Mol. Ther., 2005, 11: 452-459), and other HEK293T-based producer cell lines (e.g., Stewart et al., Hum Gene Ther. 2011, 22(3):357-369; Lee et al., Biotechnol Bioeng, 2012, 109(6): 1551-1560; Throm et al., Blood. 2009, 113(21): 5104-5110; the contents of each of which are incorporated herein by reference in their entirety).

In some aspects, the envelope proteins may be heterologous envelop proteins from other viruses, such as the G protein of vesicular stomatitis virus (VSV G) or baculoviral gp64 envelop proteins. The VSV-G glycoprotein may especially be chosen among species classified in the vesiculovirus genus: Carajas virus (CJSV), Chandipura virus (CHPV), Cocal virus (COCV), Isfahan virus (ISFV), Maraba virus (MARAV), Piry virus (PIRYV), Vesicular stomatitis Alagoas virus (VSAV), Vesicular stomatitis Indiana virus (VSIV) and Vesicular stomatitis New Jersey virus (VSNJV) and/or stains provisionally classified in the vesiculovirus genus as Grass carp rhabdovirus, BeAn 157575 virus (BeAn 157575), Boteke virus (BTKV), Calchaqui virus (CQIV), Eel virus American (EVA), Gray Lodge virus (GLOV), Jurona virus (JURY), Klamath virus (KLAV), Kwatta virus (KWAV), La Joya virus (LJV), Malpais Spring virus (MSPV), Mount Elgon bat virus (MEBV), Perinet virus (PERV), Pike fry rhabdovirus (PFRV), Porton virus (PORV), Radi virus (RADIV), Spring viremia of carp virus (SVCV), Tupaia virus (TUPV), Ulcerative disease rhabdovirus (UDRV) and Yug Bogdanovac virus (YBV). The gp64 or other baculoviral env protein can be derived from Autographa californica nucleopolyhedrovirus (AcMNPV), Anagrapha falcifera nuclear polyhedrosis virus, Bombyx mori nuclear polyhedrosis virus, Choristoneura fumiferana nucleopolyhedrovirus, Orgyia pseudotsugata single capsid nuclear polyhedrosis virus, Epiphyas postvittana nucleopolyhedrovirus, Hyphantria cunea nucleopolyhedrovirus, Galleria mellonella nuclear polyhedrosis virus, Dhori virus, Thogoto virus, Antheraea pemyi nucleopolyhedrovirus, or Batken virus.

Other elements provided in lentiviral particles may comprise retroviral LTR (long-terminal repeat) at either 5′ or 3′ terminus, a retroviral export element, optionally a lentiviral reverse response element (RRE), a promoter or active portion thereof, and a locus control region (LCR) or active portion thereof. The effector module is linked to the vector.

Methods for generating recombinant lentiviral particles are discussed in the art, for example, U.S. Pat. Nos. 8,846,385; 7,745,179; 7,629,153; 7,575,924; 7,179, 903; and 6,808,905; the contents of each of which are incorporated herein by reference in their entirety.

Lentiviral vehicles are plasmid-based or virus-based and are known in the art (See, U.S. Pat. Nos. 9,260,725; 9,068,199; 9,023,646; 8,900,858; 8,748,169; 8,709,799; 8,420,104; 8,329,462; 8,076,106; 6,013,516; and 5,994,136; the contents of each of which are incorporated herein by reference in their entirety.)

Adeno-Associated Viral Particles

Delivery of any of the pharmaceutical or therapeutic compositions of the present disclosure may be achieved using recombinant adeno-associated viral (rAAV) vectors. Such vectors or viral particles may be designed to utilize any of the known serotype capsids or combinations of serotype capsids. Capsids may include but not limited to AAV1, AAV2, AAV2G9, AAV3, AAV3a, AAV3b, AAV3-3, AAV4, AAV4-4, AAV5, AAV6, AAV6.1, AAV6.2, AAV6.1.2, AAV7, AAV7.2, AAV8, AAV9, AAV9.11, AAV9.13, AAV9.16, AAV9.24, AAV9.45, AAV9.47, AAV9.61, AAV9.68, AAV9.84, AAV9.9, AAV10, AAV11, AAV12, AAV16.3, AAV24.1, AAV27.3, AAV42.12, AAV42-1b, AAV42-2, AAV42-3a, AAV42-3b, AAV42-4, AAV42-5a, AAV42-5b, AAV42-6b, AAV42-8, AAV42-10, AAV42-11, AAV42-12, AAV42-13, AAV42-15, AAV42-aa, AAV43-1, AAV43-12, AAV43-20, AAV43-21, AAV43-23, AAV43-25, AAV43-5, AAV44.1, AAV44.2, AAV44.5, AAV223.1, AAV223.2, AAV223.4, AAV223.5, AAV223.6, AAV223.7, AAV1-7/rh.48, AAV1-8/rh.49, AAV2-15/rh.62, AAV2-3/rh.61, AAV2-4/rh.50, AAV2-5/rh.51, AAV3.1/hu.6, AAV3.1/hu.9, AAV3-9/rh.52, AAV3-11/rh.53, AAV4-8/r11.64, AAV4-9/rh.54, AAV4-19/rh.55, AAV5-3/rh.57, AAV5-22/rh.58, AAV7.3/hu.7, AAV16.8/hu.10, AAV16.12/hu.11, AAV29.3/bb.1, AAV29.5/bb.2, AAV106.1/hu.37, AAV114.3/hu.40, AAV127.2/hu.41, AAV127.5/hu.42, AAV128.3/hu.44, AAV130.4/hu.48, AAV145.1/hu.53, AAV145.5/hu.54, AAV145.6/hu.55, AAV161.10/hu.60, AAV161.6/hu.61, AAV33.12/hu.17, AAV33.4/hu.15, AAV33.8/hu.16, AAV52/hu.19, AAV52.1/hu.20, AAV58.2/hu.25, AAVA3.3, AAVA3.4, AAVA3.5, AAVA3.7, AAVC1, AAVC2, AAVC5, AAV-DJ, AAV-DJ8, AAVF3, AAVF5, AAVH2, AAVH6, AAVLK03, AAVH-1/hu.1, AAVH-5/hu.3, AAVLG-10/rh.40, AAVLG-4/rh.38, AAVLG-9/hu.39, AAVN721-8/rh.43, AAVCh.5, AAVCh.5R1, AAVcy.2, AAVcy.3, AAVcy.4, AAVcy.5, AAVCy.5R1, AAVCy.5R2, AAVCy.5R3, AAVCy.5R4, AAVcy.6, AAVhu.1, AAVhu.2, AAVhu.3, AAVhu.4, AAVhu.5, AAVhu.6, AAVhu.7, AAVhu.9, AAVhu.10, AAVhu.11, AAVhu.13, AAVhu.15, AAVhu.16, AAVhu.17, AAVhu.18, AAVhu.20, AAVhu.21, AAVhu.22, AAVhu.23.2, AAVhu.24, AAVhu.25, AAVhu.27, AAVhu.28, AAVhu.29, AAVhu.29R, AAVhu.31, AAVhu.32, AAVhu.34, AAVhu.35, AAVhu.37, AAVhu.39, AAVhu.40, AAVhu.41, AAVhu.42, AAVhu.43, AAVhu.44, AAVhu.44R1, AAVhu.44R2, AAVhu.44R3, AAVhu.45, AAVhu.46, AAVhu.47, AAVhu.48, AAVhu.48R1, AAVhu.48R2, AAVhu.48R3, AAVhu.49, AAVhu.51, AAVhu.52, AAVhu.54, AAVhu.55, AAVhu.56, AAVhu.57, AAVhu.58, AAVhu.60, AAVhu.61, AAVhu.63, AAVhu.64, AAVhu.66, AAVhu.67, AAVhu.14/9, AAVhu.t 19, AAVrh.2, AAVrh.2R, AAVrh.8, AAVrh.8R, AAVrh.10, AAVrh.12, AAVrh.13, AAVrh.13R, AAVrh.14, AAVrh.17, AAVrh.18, AAVrh.19, AAVrh.20, AAVrh.21, AAVrh.22, AAVrh.23, AAVrh.24, AAVrh.25, AAVrh.31, AAVrh.32, AAVrh.33, AAVrh.34, AAVrh.35, AAVrh.36, AAVrh.37, AAVrh.37R2, AAVrh.38, AAVrh.39, AAVrh.40, AAVrh.46, AAVrh.48, AAVrh.48.1, AAVrh.48.1.2, AAVrh.48.2, AAVrh.49, AAVrh.51, AAVrh.52, AAVrh.53, AAVrh.54, AAVrh.56, AAVrh.57, AAVrh.58, AAVrh.61, AAVrh.64, AAVrh.64R1, AAVrh.64R2, AAVrh.67, AAVrh.73, and/or AAVrh.74.

AAV vectors include not only single-stranded vectors but self-complementary AAV vectors (scAAVs). scAAV vectors contain DNA which anneals together to form double-stranded vector genome. By skipping second strand synthesis, scAAVs allow for rapid expression in the cell.

The rAAV vectors may be manufactured by standard methods in the art such as by triple transfection, in sf9 insect cells or in suspension cell cultures of human cells such as HEK293 cells.

The pharmaceutical or therapeutic compositions may be encoded in one or more viral genomes to be packaged in the AAV capsids taught herein.

Such vector or viral genomes may also include, in addition to at least one or two ITRs (inverted terminal repeats), certain regulatory elements necessary for expression from the vector or viral genome. Such regulatory elements are well known in the art and include, for example, promoters, introns, spacers, stuffer sequences, and the like.

The pharmaceutical or therapeutic compositions of the disclosure may be administered in one or more AAV particles.

In some aspects, the pharmaceutical or therapeutic compositions may be administered in one or more AAV particles. In some aspects, more than one pharmaceutical or therapeutic composition may be encoded in a viral genome.

Retroviral Vehicles/Particles (γ-Retroviral Vectors)

In some aspects, retroviral vehicles/particles may be used to deliver the pharmaceutical or therapeutic compositions. Retroviral vectors (RVs) allow the permanent integration of a transgene in target cells. In addition to lentiviral vectors based on complex HIV-1/2, retroviral vectors based on simple gamma-retroviruses have been widely used to deliver therapeutic genes and demonstrated clinically as one of the most efficient and powerful gene delivery systems capable of transducing a broad range of cell types. Example species of Gamma retroviruses include the murine leukemia viruses (MLVs) and the feline leukemia viruses (FeLV).

In some aspects, gamma-retroviral vectors derived from a mammalian gamma-retrovirus such as murine leukemia viruses (MLVs) are recombinant. The MLV families of gammaretroviruses include the ecotropic, amphotropic, xenotropic, and polytropic subfamilies. Ecotropic viruses can infect only murine cells using mCAT-1 receptor. Examples of ecotopic viruses are Moloney MLV and AKV. Amphotropic viruses infect murine, human, and other species through the Pit-2 receptor. One example of an amphotropic virus is the 4070A virus. Xenotropic and polytropic viruses utilize the same (Xpr1) receptor but differ in their species tropism. Xenotropic viruses such as NZB-9-1 infect humans and other species but not murine species, whereas polytropic viruses such as focus-forming viruses (MCF) infect murine, humans, and other species.

Gamma-retroviral vectors may be produced in packaging cells by co-transfecting the cells with several plasmids, including one encoding the retroviral structural and enzymatic (gag-pol) polyprotein, one encoding the envelope (env) protein, and one encoding the vector mRNA comprising at least one polynucleotide encoding the compositions of the present disclosure that is to be packaged in newly formed viral particles.

In some aspects, the recombinant gamma-retroviral vectors are pseudotyped with envelope proteins from other viruses. Envelope glycoproteins are incorporated in the outer lipid layer of the viral particles, which can increase/alter the cell tropism. Exemplary envelop proteins include the gibbon ape leukemia virus envelope protein (GALV) or vesicular stomatitis virus G protein (VSV-G), or Simian endogenous retrovirus envelop protein, or Measles Virus H and F proteins, or Human immunodeficiency virus gp120 envelop protein, or cocal vesiculovirus envelop protein (See, e.g., U.S. Application Publication No.: 2012/164118; the contents of which are incorporated herein by reference in its entirety). In other aspects, envelope glycoproteins may be genetically modified to incorporate targeting/binding ligands into gamma-retroviral vectors, binding ligands including, but not limited to, peptide ligands, single chain antibodies, and growth factors (Waehler et al., Nat. Rev. Genet. 2007, 8(8):573-587; the contents of which are incorporated herein by reference in its entirety). These engineered glycoproteins can retarget vectors to cells expressing their corresponding target moieties. In other aspects, a “molecular bridge” may be introduced to direct vectors to specific cells. The molecular bridge has dual specificities: one end can recognize viral glycoproteins, and the other end can bind to the molecular determinant on the target cell. Such molecular bridges, for example, ligand-receptor, avidin-biotin, and chemical conjugations, monoclonal antibodies, and engineered fusogenic proteins, can direct the attachment of viral vectors to target cells for transduction (Yang et al., Biotechnol. Bioeng., 2008, 101(2): 357-368; and Maetzig et al., Viruses, 2011, 3, 677-713; the contents of each of which are incorporated herein by reference in their entirety).

In some aspects, the recombinant gamma-retroviral vectors are self-inactivating (SIN) gammaretroviral vectors. The vectors are replication incompetent. SIN vectors may harbor a deletion within the 3′ U3 region initially comprising enhancer/promoter activity. Furthermore, the 5′ U3 region may be replaced with strong promoters (needed in the packaging cell line) derived from Cytomegalovirus or RSV, or an internal promotor of choice, and/or an enhancer element. The choice of the internal promotors may be made according to specific requirements of gene expression needed for a particular purpose of the disclosure.

In some aspects, polynucleotides encoding the pharmaceutical or therapeutic compositions are inserted within the recombinant viral genome. The other components of the viral mRNA of a recombinant gamma-retroviral vector may be modified by insertion or removal of naturally occurring sequences (e.g., insertion of an IRES, insertion of a heterologous polynucleotide encoding a polypeptide or inhibitory nucleic acid of interest, shuffling of a more effective promoter from a different retrovirus or virus in place of the wild-type promoter and the like). In some examples, the recombinant gamma-retroviral vectors may comprise modified packaging signal, and/or primer binding site (PBS), and/or 5′-enhancer/promoter elements in the U3-region of the 5′-long terminal repeat (LTR), and/or 3′-SIN elements modified in the U3-region of the 3′-LTR. These modifications may increase the titers and the ability of infection.

Gammaretroviral vectors suitable for pharmaceutical or therapeutic compositions, of the present disclosure, may be selected from those disclosed in U.S. Pat. Nos. 8,828,718; 7,585,676; 7,351,585; U.S. Application Publication No.: 2007/048285; PCT Application Publication Nos.: WO2010/113037; WO2014/121005; WO2015/056014; and EP Pat. Nos.: EP1757702; EP1757703 (the contents of each of which are incorporated herein by reference in their entirety).

Messenger RNA (mRNA)

In some aspects, the pharmaceutical or therapeutic compositions may be designed as a messenger RNA (mRNA). As used herein, the term “messenger RNA” (mRNA) refers to any polynucleotide that encodes a polypeptide of interest and is capable of being translated to produce the encoded polypeptide in vitro, in vivo, in situ, or ex vivo. Such mRNA molecules of the present disclosure may have the structural components or features of any of those taught in International Application Number PCT/US2013/030062, the contents of which are incorporated herein by reference in its entirety.

Pharmaceutical compositions, e.g., mRNA vaccines or mRNA vaccine cassettes of the present disclosure, may also be designed as taught in, for example, Ribostem Limited in United Kingdom patent application serial number 0316089.2 filed on Jul. 9, 2003, now abandoned, PCT application number PCT/GB2004/002981 filed on Jul. 9, 2004, published as WO2005005622, U.S. patent application Ser. No. 10/563,897 filed on Jun. 8, 2006, published as US20060247195 now abandoned, and European patent application national phase entry serial number EP2004743322 filed on Jul. 9, 2004, published as EP1646714 now withdrawn; Novozymes, Inc. in PCT application number PCT/US2007/88060 filed on Dec. 19, 2007, published as WO2008140615, United States patent application national phase entry Ser. No. 12/520,072 filed on Jul. 2, 2009, published as US20100028943 and European patent application number EP2007874376 filed on Jul. 7, 2009, published as EP2104739; University of Rochester in PCT application number PCT/US2006/46120 filed on Dec. 4, 2006, published as WO2007064952 and U.S. patent application Ser. No. 11/606,995 filed on Dec. 1, 2006, published as US20070141030; BioNTech AG in European patent application serial number EP2007024312 filed Dec. 14, 2007, now abandoned, PCT application number PCT/EP2008/01059 filed on Dec. 12, 2008, published as WO2009077134, European patent application number EP2008861423 filed on Jun. 2, 2010 published as EP2240572, U.S. patent application Ser. No. 12/735,060 filed Nov. 24, 2010, published as US20110065103, German patent application number DE 10 2005 046 490 filed Sep. 28, 2005, PCT application PCT/EP2006/0448 filed Sep. 28, 2006, published as WO2007036366, European patent EP1934345, published Mar. 21, 2012 and U.S. patent application Ser. No. 11/992,638 filed Aug. 14, 2009, published as 20100129877; Immune Disease Institute Inc. in U.S. patent application Ser. No. 13/088,009 filed Apr. 15, 2011, published as US20120046346 and PCT application PCT/US2011/32679 filed Apr. 15, 2011, published as WO20110130624; Shire Human Genetic Therapeutics in U.S. patent application Ser. No. 12/957,340 filed on Nov. 20, 2010, published as US20110244026; Sequitur Inc. in PCT application PCT/US1998/019492 filed on Sep. 18, 1998, published as WO1999014346; The Scripps Research Institute in PCT application number PCT/US2010/00567 filed on Feb. 24, 2010, published as WO2010098861, and U.S. patent application Ser. No. 13/203,229 filed Nov. 3, 2011, published as US20120053333; Ludwig-Maximillians University in PCT application number PCT/EP2010/004681 filed on Jul. 30, 2010, published as WO2011012316; Cellscript Inc. in U.S. Pat. No. 8,039,214 filed Jun. 30, 2008 and granted Oct. 18, 2011, U.S. patent application Ser. No. 12/962,498 filed on Dec. 7, 2010, published as US20110143436, Ser. No. 12/962,468 filed on Dec. 7, 2010, published as US20110143397, Ser. No. 13/237,451 filed on Sep. 20, 2011, published as US20120009649, and PCT applications PCT/US2010/59305 filed Dec. 7, 2010, published as WO2011071931 and PCT/US2010/59317 filed on Dec. 7, 2010, published as WO2011071936; The Trustees of the University of Pennsylvania in PCT application number PCT/US2006/32372 filed on Aug. 21, 2006, published as WO2007024708, and U.S. patent application Ser. No. 11/990,646 filed on Mar. 27, 2009, published as US20090286852; Curevac GMBH in German patent application serial numbers DE10 2001 027 283.9 filed Jun. 5, 2001, DE10 2001 062 480.8 filed Dec. 19, 2001, and DE 20 2006 051 516 filed Oct. 31, 2006 all abandoned, European patent numbers EP1392341 granted Mar. 30, 2005 and EP1458410 granted Jan. 2, 2008, PCT application numbers PCT/EP2002/06180 filed Jun. 5, 2002, published as WO2002098443, PCT/EP2002/14577 filed on Dec. 19, 2002, published as WO2003051401, PCT/EP2007/09469 filed on Dec. 31, 2007, published as WO2008052770, PCT/EP2008/03033 filed on Apr. 16, 2008, published as WO2009127230, PCT/EP2006/004784 filed on May 19, 2005, published as WO2006122828, PCT/EP2008/00081 filed on Jan. 9, 2007, published as WO2008083949, and U.S. patent application Ser. No. 10/729,830 filed on Dec. 5, 2003, published as US20050032730, Ser. No. 10/870,110 filed on Jun. 18, 2004, published as US20050059624, Ser. No. 11/914,945 filed on Jul. 7, 2008, published as US20080267873, Ser. No. 12/446,912 filed on Oct. 27, 2009, published as US2010047261 now abandoned, Ser. No. 12/522,214 filed on Jan. 4, 2010, published as US20100189729, Ser. No. 12/787,566 filed on May 26, 2010, published as US20110077287, Ser. No. 12/787,755 filed on May 26, 2010, published as US20100239608, Ser. No. 13/185,119 filed on Jul. 18, 2011, published as US20110269950, and Ser. No. 13/106,548 filed on May 12, 2011, published as US20110311472 all of which are herein incorporated by reference in their entirety.

Naked Delivery

Pharmaceutical or therapeutic compositions of the present disclosure may be delivered to cells, tissues, organs and/or organisms in naked form. As used herein in, the term “naked” refers to pharmaceutical or therapeutic compositions delivered free from agents or modifications which promote transfection or permeability. The naked pharmaceutical or therapeutic compositions may be delivered to the cells, tissues, organs and/or organisms using routes of administration known in the art and described herein. In some aspects, naked delivery may include formulation in a simple buffer such as saline or PBS.

Formulated Delivery

The compositions of the present disclosure may be formulated by any method known in the art.

In some aspects, pharmaceutical or therapeutic compositions of the present disclosure may be formulated using methods described herein.

Formulations may comprise pharmaceutical or therapeutic compositions which may be modified and/or unmodified.

Formulations may further include, but are not limited to, cell penetration agents, pharmaceutically acceptable carriers, delivery agents, bioerodible or biocompatible polymers, solvents, and/or sustained-release delivery depots. Formulations of the present disclosure may be delivered to cells using routes of administration known in the art and described herein.

Pharmaceutical or therapeutic compositions may also be formulated for direct delivery to organs or tissues in any of several ways in the art including, but not limited to, direct soaking or bathing, via a catheter, by gels, powder, ointments, creams, gels, lotions, and/or drops, by using substrates such as fabric or biodegradable materials coated or impregnated with compositions, and the like.

In one example, the composition described herein is an RNA-based (e.g., mRNA) nanoparticle pharmaceutical or therapeutic composition. The nanoparticle may comprise the polynucleotide described being encapsulated by a delivery vehicle molecule that has a formulation that may be, but not limited to, poly(lactic-co-glycolic acid)(PLGA) microspheres, lipidoids, lipoplex, liposome, polymers, carbohydrates (including simple sugars), cationic lipids, and combinations thereof.

In one aspect, the delivery vehicle molecule formulation may comprise at least one lipid. The lipid may be selected from, but is not limited to, DLin-DMA, DLin-K-DMA, 98N12-5, C12-200, DLin-MC3-DMA, DLin-KC2-DMA, DODMA, PLGA, PEG, PEG-DMG, and PEGylated lipids. In another aspect, the lipid may be a cationic lipid such as, but not limited to, DLin-DMA, DLin-D-DMA, DLin-MC3-DMA, DLin-KC2-DMA, and DODMA.

In one aspect, the delivery vehicle molecule may have a geometry of a nanoparticle. The delivery vehicle may be, for example, an amino lipidated peptide that may include tertiary amino lipidated cationic peptides, such as any of those described in PCT application, PCT/US19/53661, titled “LIPID NANOPARTICLE FORMULATIONS COMPRISING LIPIDATED CATIONIC PEPTIDE COMPOUNDS FOR NUCLEIC ACID DELIVERY”, filed on Sep. 27, 2019, and in PCT/US19/53655, titled “TERTIARY AMINO LIPIDATED CATIONIC PEPTIDES FOR NUCLEIC ACID DELIVERY” filed on Sep. 27, 2019, the contents of each of which are incorporated herein by reference in their entirety. The nanoparticle delivery vehicle may comprise additional lipids/components. For example, the amino lipidated peptides can include one or more phospholipids, e.g., MSPC or DSPC. The lipid composition can also comprise a quaternary amine compound such as DOTAP.

The pharmaceutical or therapeutic compositions may be formulated using any of the delivery vehicles taught in, for example, US Publication No. US20180028688, the contents of which are incorporated herein by reference in their entirety.

Detectable Agents and Labels

The pharmaceutical or therapeutic compositions of the present disclosure may be associated with or bound to one or more ratioactive agents or detectable agents. These agents include various organic small molecules, inorganic compounds, nanoparticles, enzymes or enzyme substrates, fluorescent materials, luminescent materials (e.g., luminol), bioluminescent materials (e.g., luciferase, luciferin, and aequorin), chemiluminescent materials, radioactive materials (e.g., ¹⁸F, ⁶⁷Ga, ^{81m}Kr, ⁸²Rb, ¹¹¹In, ¹²³I, ¹³³Xe, ²⁰¹Tl, ¹²⁵I, ³⁵⁵S, ¹⁴C, ³H, or ^{99m}Tc (e.g., as pertechnetate (technetate (VII), TcO₄⁻)), and contrast agents (e.g., gold (e.g., gold nanoparticles), gadolinium (e.g., chelated Gd), iron oxides (e.g., superparamagnetic iron oxide (SPIO), monocrystalline iron oxide nanoparticles (MIONs), and ultrasmall superparamagnetic iron oxide (USPIO)), manganese chelates (e.g., Mn-DPDP), barium sulfate, iodinated contrast media (iohexol), microbubbles, or perfluorocarbons. Such optically-detectable labels include for example, without limitation, 4-acetamido-4′-isothiocyanatostilbene-2,2′disulfonic acid; acridine and derivatives (e.g., acridine and acridine isothiocyanate); 5-(2′-aminoethyl)aminonaphthalene-1-sulfonic acid (EDANS); 4-amino-N-[3-vinylsulfonyl)phenyl] naphthalimide-3,5 disulfonate; N-(4-anilino-1-naphthyl)maleimide; anthranilamide; BODIPY; Brilliant Yellow; coumarin and derivatives (e.g., coumarin, 7-amino-4-methylcoumarin (AMC, Coumarin 120), and 7-amino trifluoromethylcoumarin (Coumarin 151)); cyanine dyes; cyanosine; 4′,6-diaminidino-2-phenylindole (DAPI); 5′ 5″-dibromopyrogallol-sulfonaphthalein (Bromopyrogallol Red); 7-diethylamino-3-(4′-isothiocyanatophenyl)-4-methylcoumarin; diethylenetriamine pentaacetate; 4,4′-diisothiocyanatodihydro-stilbene-2,2′-disulfonic acid; 4,4′-diisothiocyanatostilbene-2,2 ‘-disulfonic acid; 5-[dimethylamino]-naphthalene-1-sulfonyl chloride (DNS, dansylchloride); 4-dimethylaminophenylazophenyl-4′-isothiocyanate (DAB ITC); eosin and derivatives (e.g., eosin and eosin isothiocyanate); erythrosin and derivatives (e.g., erythrosin B and erythrosin isothiocyanate); ethidium; fluorescein and derivatives (e.g., 5-carboxyfluorescein (FAM), 5-(4,6-dichlorotriazin-2-yl)aminofluorescein (DTAF), 2′,7′-dimethoxy-4′5′-dichloro-6-carboxyfluorescein, fluorescein, fluorescein isothiocyanate, X-rhodamine-5-(and-6)-isothiocyanate (QFITC or XRITC), and fluorescamine); 2-[2-[3-[[1,3-dihydro-1,1-dimethyl-3-(3-sulfopropyl)-2H-benz [e]indol-2-ylidene]ethylidene]-2-[4-(ethoxycarbonyl)-1-piperazinyl]-1-cyclopenten-1-yl]ethenyl]-1,1-dimethyl-3-(3-sulforpropyl)-1H-benz [e]indolium hydroxide, inner salt, compound with n,n-diethylethanamine (1:1) (IR144); 5-chloro-2-[2-[3-[(5-chloro-3-ethyl-2(3H)-benzothiazol-ylidene)ethylidene]-2-(diphenylamino)-1-cyclopenten-1-yl]ethenyl]-3-ethyl benzothiazolium perchlorate (IR140); Malachite Green isothiocyanate; 4-methylumbelliferone orthocresolphthalein; nitrotyrosine; pararosaniline; Phenol Red; B-phycoerythrin; o-phthaldialdehyde; pyrene and derivatives (e.g., pyrene, pyrene butyrate, and succinimidyl 1-pyrene); butyrate quantum dots; Reactive Red 4 (CIBACRON™ Brilliant Red 3B-A); rhodamine and derivatives (e.g., 6-carboxy-X-rhodamine (ROX), 6-carboxyrhodamine (R6G), lissamine rhodamine B sulfonyl chloride rhodarnine (Rhod), rhodamine B, rhodamine 123, rhodamine X isothiocyanate, sulforhodamine B, sulforhodamine 101, sulfonyl chloride derivative of sulforhodamine 101 (Texas Red), N,N,N′,N′tetramethyl-6-carboxyrhodamine (TAMRA) tetramethyl rhodamine, and tetramethyl rhodamine isothiocyanate (TRITC)); riboflavin; rosolic acid; terbium chelate derivatives; Cyanine-3 (Cy3); Cyanine-5 (Cy5); cyanine-5.5 (Cy5.5), Cyanine-7 (Cy7); IRD 700; IRD 800; Alexa 647; La Jolta Blue; phthalo cyanine; and naphthalo cyanine.

In some aspects, the detectable agent may be a non-detectable precursor that becomes detectable upon activation (e.g., fluorogenic tetrazine-fluorophore constructs (e.g., tetrazine-BODIPY FL, tetrazine-Oregon Green 488, or tetrazine-BODIPY TMR-X) or enzyme activatable fluorogenic agents (e.g., PROSENSE® (VisEn Medical))). In vitro assays in which the enzyme labeled compositions can be used include, but are not limited to, enzyme linked immunosorbent assays (ELISAs), immunoprecipitation assays, immunofluorescence, enzyme immunoassays (EIA), radioimmunoassays (RIA), and Western blot analysis.

Kits

The present disclosure includes a variety of kits for conveniently and/or effectively carrying out methods of the present disclosure. Typically, kits will comprise sufficient amounts and/or numbers of components to allow a user to perform one or multiple treatments of a subject(s) and/or to perform one or multiple experiments.

In one aspect, the present disclosure provides kits for inducing an immune response in a subject or patient, optionally in combination with any other suitable active agents.

The kit may further comprise packaging and instructions and/or a delivery agent to form a formulation composition. The delivery agent may comprise, for example, saline, a buffered solution.

In additional aspects, assay screening kits are provided. The kit includes a container for the screening assay. Instructions for the use of the assay and the information about the screening method are to be included in the kit.

### Terminology

Nucleotides are referred to by their commonly accepted single-letter codes. Unless otherwise indicated, nucleic acids are written left to right in 5′ to 3′ orientation. Nucleotides are referred to herein by their commonly known one-letter symbols recommended by the IUPAC-IUB Biochemical Nomenclature Commission. Accordingly, A represents adenine, C represents cytosine, G represents guanine, T represents thymine, and U represents uracil.

Amino acids are referred to herein by either their commonly known three letter symbols or by the one-letter symbols recommended by the IUPAC-IUB Biochemical Nomenclature Commission. Unless otherwise indicated, amino acid sequences are written left to right in amino to carboxy orientation.

About: The term “about” as used in connection with a numerical value throughout the specification and the claims denotes an interval of accuracy, familiar and acceptable to a person skilled in the art. In general, such interval of accuracy is +10%.

Where ranges are given, endpoints are included. Furthermore, unless otherwise indicated or otherwise evident from the context and understanding of one of ordinary skill in the art, values that are expressed as ranges can assume any specific value or subrange within the stated ranges in different aspects of the disclosure, to the tenth of the unit of the lower limit of the range, unless the context clearly dictates otherwise.

Administered in combination: As used herein, the term “administered in combination,” “concurrent administration,” combined administration,” or “combination therapy” means that two or more agents are administered to a subject at the same time or within an interval such that there can be an overlap of an effect of each agent on the patient. In some aspects, they are administered within about 60, 30, 15, 10, 5, or 1 minute of one another. In some aspects, the administrations of the agents are spaced sufficiently close together such that a combinatorial (e.g., a synergistic) effect is achieved.

Amino acid substitution: The term “amino acid substitution” refers to replacing an amino acid residue present in a parent or reference sequence (e.g., a wild type sequence) with another amino acid residue. An amino acid can be substituted in a parent or reference sequence (e.g., a wild type polypeptide sequence), for example, via chemical peptide synthesis or through recombinant methods known in the art. Accordingly, a reference to a “substitution at position X” refers to the substitution of an amino acid present at position X with an alternative amino acid residue. In some aspects, substitution patterns can be described according to the schema AnY, wherein A is the single letter code corresponding to the amino acid naturally or originally present at position n, and Y is the substituting amino acid residue. In other aspects, substitution patterns can be described according to the schema An(YZ), wherein A is the single letter code corresponding to the amino acid residue substituting the amino acid naturally or originally present at position X, and Y and Z are alternative substituting amino acid residue.

In the context of the present disclosure, substitutions (even when they referred to as an amino acid substitution) are conducted at the nucleic acid level, i.e., substituting an amino acid residue with an alternative amino acid residue is conducted by substituting the codon encoding the first amino acid with a codon encoding the second amino acid.

Animal: As used herein, the term “animal” refers to any member of the animal kingdom. In some aspects, “animal” refers to humans at any stage of development. In some aspects, “animal” refers to non-human animals at any stage of development. In certain aspects, the non-human animal is a mammal (e.g., a rodent, a mouse, a rat, a rabbit, a monkey, a dog, a cat, a sheep, cattle, a primate, or a pig). In some aspects, animals include, but are not limited to, mammals, birds, reptiles, amphibians, fish, and worms. In some aspects, the animal is a transgenic animal, genetically engineered animal, or a clone.

Antigens of interest or desired antigens: As used herein, the terms “antigens of interest” or “desired antigens” or “antigens” refers to those proteins and/or other biomolecules which elicit an immune response, e.g., the production of antibodies. In some aspects, antigens of interest may comprise any of the polypeptides or payloads or proteins described herein, or fragments or portions thereof.

Approximately: As used herein, the term “approximately,” as applied to one or more values of interest, refers to a value that is similar to a stated reference value. In certain aspects, the term “approximately” refers to a range of values that fall within 10%, 9%, 8%, 7%, 6%, 5%, 4%, 3%, 2%, 1%, or less in either direction (greater than or less than) of the stated reference value unless otherwise stated or otherwise evident from the context (except where such number would exceed 100% of a possible value).

Associated with: As used herein with respect to a disease, the term “associated with” means that the symptom, measurement, characteristic, or status in question is linked to the diagnosis, development, presence, or progression of that disease. As association may, but need not, be causatively linked to the disease.

The terms “associated with,” “conjugated,” “linked,” “attached,” and “tethered,” when used with respect to two or more moieties, means that the moieties are physically associated or connected with one another, either directly or via one or more additional moieties that serves as a linking agent, to form a structure that is sufficiently stable so that the moieties remain physically associated under the conditions in which the structure is used, e.g., physiological conditions. An “association” need not be strictly through direct covalent chemical bonding. It may also suggest ionic or hydrogen bonding or a hybridization-based connectivity sufficiently stable such that the “associated” entities remain physically associated.

Biocompatible: As used herein, the term “biocompatible” means compatible with living cells, tissues, organs, or systems posing little to no risk of injury, toxicity, or rejection by the immune system.

Biodegradable: As used herein, the term “biodegradable” means capable of being broken down into innocuous products by the action of living things.

Sequence Optimization: The term “sequence optimization” refers to a process or series of processes by which nucleobases in a reference nucleic acid sequence are replaced with alternative nucleobases, resulting in a nucleic acid sequence with improved properties, e.g., improved protein expression or immunogenicity.

In general, the goal in sequence optimization is to produce a synonymous nucleotide sequence that encodes the same polypeptide sequence encoded by the reference nucleotide sequence. Thus, there are no amino acid substitutions (as a result of codon optimization) in the polypeptide encoded by the codon optimized nucleotide sequence with respect to the polypeptide encoded by the reference nucleotide sequence.

Codon substitution: The terms “codon substitution” or “codon replacement” in the context of sequence optimization refer to replacing a codon present in a reference nucleic acid sequence with another codon. A codon can be substituted in a reference nucleic acid sequence, for example, via chemical peptide synthesis or through recombinant methods known in the art. Accordingly, references to a “substitution” or “replacement” at a certain location in a nucleic acid sequence (e.g., an mRNA) or within a certain region or subsequence of a nucleic acid sequence (e.g., an mRNA) refer to the substitution of a codon at such location or region with an alternative codon.

As used herein, the terms “coding region” and “region encoding” and grammatical variants thereof, refer to an Open Reading Frame (ORF) in a polynucleotide that upon expression yields a polypeptide or protein.

Compound: As used herein, the term “compound,” is meant to include all stereoisomers and isotopes of the structure depicted. As used herein, the term “stereoisomer” means any geometric isomer (e.g., cis- and trans-isomer), enantiomer, or diastereomer of a compound. The present disclosure encompasses any and all stereoisomers of the compounds described herein, including stereomerically pure forms (e.g., geometrically pure, enantiomerically pure, or diastereomerically pure) and enantiomeric and stereoisomeric mixtures, e.g., racemates. Enantiomeric and stereomeric mixtures of compounds and means of resolving them into their component enantiomers or stereoisomers are well-known. “Isotopes” refers to atoms having the same atomic number but different mass numbers resulting from a different number of neutrons in the nuclei. For example, isotopes of hydrogen include tritium and deuterium. Further, a compound, salt, or complex of the present disclosure can be prepared in combination with solvent or water molecules to form solvates and hydrates by routine methods.

Conservative amino acid substitution: A “conservative amino acid substitution” is one in which the amino acid residue is replaced with an amino acid residue having a similar side chain. Families of amino acid residues having similar side chains have been defined in the art, including basic side chains (e.g., lysine, arginine, or histidine), acidic side chains (e.g., aspartic acid or glutamic acid), uncharged polar side chains (e.g., glycine, asparagine, glutamine, serine, threonine, tyrosine, or cysteine), nonpolar side chains (e.g., alanine, valine, leucine, isoleucine, proline, phenylalanine, methionine, or tryptophan), beta-branched side chains (e.g., threonine, valine, isoleucine) and aromatic side chains (e.g., tyrosine, phenylalanine, tryptophan, or histidine). Thus, if an amino acid in a polypeptide is replaced with another amino acid from the same side chain family, the amino acid substitution is considered to be conservative. In another aspect, a string of amino acids can be conservatively replaced with a structurally similar string that differs in order and/or composition of side chain family members.

Non-conservative amino acid substitutions include those in which (i) a residue having an electropositive side chain (e.g., Arg, His or Lys) is substituted for, or by, an electronegative residue (e.g., Glu or Asp), (ii) a hydrophilic residue (e.g., Ser or Thr) is substituted for, or by, a hydrophobic residue (e.g., Ala, Leu, Ile, Phe or Val), (iii) a cysteine or proline is substituted for, or by, any other residue, or (iv) a residue having a bulky hydrophobic or aromatic side chain (e.g., Val, His, Be or Trp) is substituted for, or by, one having a smaller side chain (e.g., Ala or Ser) or no side chain (e.g., Gly).

Other amino acid substitutions can be readily identified by people of ordinary skill. For example, for the amino acid alanine, a substitution can be taken from any one of D-alanine, glycine, beta-alanine, L-cysteine, and D-cysteine. For lysine, a replacement can be any one of D-lysine, arginine, D-arginine, homo-arginine, methionine, D-methionine, ornithine, or D-ornithine. Generally, substitutions in functionally important regions that can be expected to induce changes in the properties of isolated polypeptides are those in which (i) a polar residue, e.g., serine or threonine, is substituted for (or by) a hydrophobic residue, e.g., leucine, isoleucine, phenylalanine, or alanine; (ii) a cysteine residue is substituted for (or by) any other residue; (iii) a residue having an electropositive side chain, e.g., lysine, arginine or histidine, is substituted for (or by) a residue having an electronegative side chain, e.g., glutamic acid or aspartic acid; or (iv) a residue having a bulky side chain, e.g., phenylalanine, is substituted for (or by) one not having such a side chain, e.g., glycine. The likelihood that one of the foregoing non-conservative substitutions can alter functional properties of the protein is also correlated to the position of the substitution with respect to functionally important regions of the protein. Some non-conservative substitutions can accordingly have little or no effect on biological properties.

Conserved: As used herein, the term “conserved” refers to nucleotides or amino acid residues of a polynucleotide sequence or polypeptide sequence, respectively, that are those that occur unaltered in the same position of two or more sequences being compared. Nucleotides or amino acids that are relatively conserved are those that are conserved amongst more related sequences than nucleotides or amino acids appearing elsewhere in the sequences.

In some aspects, two or more sequences are said to be “completely conserved” if they are 100% identical to one another. In some aspects, two or more sequences are said to be “highly conserved” if they are at least 70% identical, at least 80% identical, at least 90% identical, or at least 95% identical to one another. In some aspects, two or more sequences are said to be “highly conserved” if they are about 70% identical, about 80% identical, about 90% identical, about 95%, about 98%, or about 99% identical to one another. In some aspects, two or more sequences are said to be “conserved” if they are at least 30% identical, at least 40% identical, at least 50% identical, at least 60% identical, at least 70% identical, at least 80% identical, at least 90% identical, or at least 95% identical to one another. In some aspects, two or more sequences are said to be “conserved” if they are about 30% identical, about 40% identical, about 50% identical, about 60% identical, about 70% identical, about 80% identical, about 90% identical, about 95% identical, about 98% identical, or about 99% identical to one another. Conservation of sequence may apply to the entire length of a polynucleotide or polypeptide or may apply to a portion, region, or feature thereof.

Contacting: As used herein, the term “contacting” means establishing a physical connection between two or more entities. For example, contacting a mammalian cell with a composition means that the mammalian cell and the composition are made to share a physical connection. Methods of contacting cells with external entities both in vivo and ex vivo are well known in the biological arts. For example, contacting a composition and a mammalian cell disposed within a mammal may be performed by varied routes of administration (e.g., intravenous, intramuscular, intradermal, and subcutaneous) and may involve varying amounts of compositions. Moreover, more than one mammalian cell may be contacted by a composition.

Controlled Release: As used herein, the term “controlled release” refers to a pharmaceutical or therapeutic composition or compound release profile that conforms to a particular pattern of release to effect a desired, e.g., therapeutic outcome.

Covalent Derivative: The term “covalent derivative” when referring to polypeptides includes modifications of a native or starting protein with an organic proteinaceous or non-proteinaceous derivatizing agent and/or post-translational modifications. Covalent modifications are traditionally introduced by reacting targeted amino acid residues of the protein with an organic derivatizing agent that is capable of reacting with selected side-chains or terminal residues or by harnessing mechanisms of post-translational modifications that function in selected recombinant host cells. The resultant covalent derivatives are useful in programs directed at identifying residues important for biological activity, for immunoassays, or for the preparation of anti-protein antibodies for immunoaffinity purification of the recombinant glycoprotein. Such modifications are within the ordinary skill in the art and are performed without undue experimentation.

Cyclic or Cyclized: As used herein, the term “cyclic” refers to the presence of a continuous loop. Cyclic molecules need not be circular, only joined to form an unbroken chain of subunits. Cyclic molecules such as the engineered RNA or mRNA can be single units or multimers or comprise one or more components of a complex or higher order structure.

Cytotoxic: As used herein, “cytotoxic” refers to killing or causing injurious, toxic, or deadly effect on a cell (e.g., a mammalian cell (e.g., a human cell)), bacterium, virus, fungus, protozoan, parasite, prion, or a combination thereof.

Delivering: As used herein, the term “delivering” means providing an entity to a destination. For example, delivering a polynucleotide to a subject may involve administering a composition to the subject (e.g., by an intravenous, intramuscular, intradermal, or subcutaneous route). Administration of a composition to a mammal or mammalian cell may involve contacting one or more cells with the composition.

Delivery Vehicle: As used herein, “delivery vehicle” refers to any substance that facilitates, at least in part, the in vivo, in vitro, or ex vivo delivery of a polynucleotide to targeted cells or tissues (e.g., tumors, etc.). Referring to something as a delivery vehicle does not mean that it may not also have therapeutic effects.

Destabilized: As used herein, the term “destable,” “destabilize,” or “destabilizing region” means a region or molecule that is less stable than a starting, wild-type, or native form of the same region or molecule.

Detectable label: As used herein, “detectable label” refers to one or more markers, signals, or moieties that are attached, incorporated, or associated with another entity that is readily detected by methods known in the art, including radiography, fluorescence, chemiluminescence, enzymatic activity, absorbance and the like. Detectable labels include radioisotopes, fluorophores, chromophores, enzymes, dyes, metal ions, ligands such as biotin, avidin, streptavidin and haptens, quantum dots, and the like. Detectable labels can be located at any position in the peptides or proteins disclosed herein. They can be within the amino acids, the peptides, or proteins, or located at the N- or C-termini.

Diastereomer: As used herein, the term “diastereomer,” means stereoisomers that are not mirror images of one another and are non-superimposable on one another.

Digest: As used herein, the term “digest” means to break apart into smaller pieces or components. When referring to polypeptides or proteins, digestion results in the production of peptides.

Distal: As used herein, the term “distal” means situated away from the center or away from a point or region of interest.

Domain: As used herein, when referring to polypeptides, the term “domain” refers to a motif of a polypeptide having one or more identifiable structural or functional characteristics or properties (e.g., binding capacity, serving as a site for protein-protein interactions).

Dosing regimen: As used herein, a “dosing regimen” or a “dosing regimen” is a schedule of administration or physician determined regimen of treatment, prophylaxis, or palliative care.

Effective Amount: As used herein, the term “effective amount” of an agent is that amount sufficient to effect beneficial or desired results, for example, clinical results, and, as such, an “effective amount” depends upon the context in which it is being applied. The term “effective amount” can be used interchangeably with “effective dose,” “therapeutically effective amount,” or “therapeutically effective dose.”

Enantiomer: As used herein, the term “enantiomer” means each individual optically active form of a compound of the disclosure, having an optical purity or enantiomeric excess (as determined by methods standard in the art) of at least 80% (i.e., at least 90% of one enantiomer and at most 10% of the other enantiomer), at least 90%, or at least 98%.

Encapsulate: As used herein, the term “encapsulate” means to enclose, surround or encase.

Engineered: As used herein, aspects of the disclosure are “engineered” when they are designed to have a feature or property, whether structural or chemical, that varies from a starting point, wild type, or native molecule.

Enhanced Delivery: As used herein, the term “enhanced delivery” means delivery of more (e.g., at least 1.5 fold more, at least 2-fold more, at least 3-fold more, at least 4-fold more, at least 5-fold more, at least 6-fold more, at least 7-fold more, at least 8-fold more, at least 9-fold more, at least 10-fold more) of a composition to a target tissue of interest (e.g., mammalian liver) compared to the level of delivery by a control composition to a target tissue of interest. The level of delivery may be measured by comparing the amount of protein produced in a tissue to the weight of said tissue, comparing the amount of polynucleotide in a tissue to the weight of said tissue, comparing the amount of protein produced in a tissue to the amount of total protein in said tissue, or comparing the amount of polynucleotide in a tissue to the amount of total polynucleotide in said tissue. It will be understood that the enhanced delivery to a target tissue need not be determined in a subject being treated, it may be determined in a surrogate such as an animal model (e.g., a rat model).

Exosome: As used herein, “exosome” is a vesicle secreted by mammalian cells.

Expression: As used herein, “expression” of a nucleic acid sequence refers to one or more of the following events: (1) production of an RNA template from a DNA sequence (e.g., by transcription); (2) processing of an RNA transcript (e.g., by splicing, editing, 5′ cap formation, and/or 3′ end processing); (3) translation of an RNA into a polypeptide or protein; and (4) post-translational modification of a polypeptide or protein.

Ex Vivo: As used herein, the term “ex vivo” refers to events that occur outside of an organism (e.g., animal, plant, or microbe or cell or tissue thereof). Ex vivo events may take place in an environment minimally altered from a natural (e.g., in vivo) environment.

Feature: As used herein, a “feature” refers to a characteristic, a property, or a distinctive element. When referring to polypeptides, “features” are defined as distinct amino acid sequence-based components of a molecule. Features of the polypeptides encoded by the polynucleotides of the present disclosure include surface manifestations, local conformational shape, folds, loops, half-loops, domains, half-domains, sites, termini, or any combination thereof.

Formulation: As used herein, a “formulation” includes at least a polynucleotide or polypeptide and one or more of a carrier, an excipient, and a delivery agent or vehicle.

Forward scatter (FSC): As used herein, forward scatter or FSC is a flow cytometry measurement that detects the light scattered by cells along the path of the laser.

Fragment: A “fragment,” as used herein, refers to a portion. For example, fragments of proteins can comprise polypeptides obtained by digesting full-length protein isolated from cultured cells. In some aspects, a fragment is a subsequence of a full-length protein (e.g., one of the subunits of IL-23) wherein N-terminal, and/or C-terminal, and/or internal subsequences have been deleted. In some preferred aspects of the present disclosure, the fragments of a protein of the present disclosure are functional fragments.

Functional: As used herein, a “functional” biological molecule is a biological molecule in a form in which it exhibits a property and/or activity by which it is characterized.

Homology: As used herein, the term “homology” refers to the overall relatedness between polymeric molecules, e.g., between nucleic acid molecules (e.g., DNA molecules and/or RNA molecules) and/or between polypeptide molecules. Generally, the term “homology” implies an evolutionary relationship between two molecules. Thus, two molecules that are homologous will have a common evolutionary ancestor. In the context of the present disclosure, the term homology encompasses both identity and similarity.

In some aspects, polymeric molecules are considered to be “homologous” to one another if at least 25%, 30%, 35%, 40%, 45%, 50%, 55%, 60%, 65%, 70%, 75%, 80%, 85%, 90%, 95%, or 99% of the monomers in the molecule are identical (exactly the same monomer) or are similar (conservative substitutions). The term “homologous” necessarily refers to a comparison between at least two sequences (polynucleotide or polypeptide sequences).

Identity: As used herein, the term “identity” refers to the overall monomer conservation between polymeric molecules, e.g., between polynucleotide molecules (e.g., DNA molecules and/or RNA molecules) and/or between polypeptide molecules. Calculation of the percent identity of two polynucleotide sequences, for example, can be performed by aligning the two sequences for optimal comparison purposes (e.g., gaps can be introduced in one or both of a first and a second nucleic acid sequence for optimal alignment and non-identical sequences can be disregarded for comparison purposes). In certain aspects, the length of a sequence aligned for comparison purposes is at least 30%, at least 40%, at least 50%, at least 60%, at least 70%, at least 80%, at least 90%, at least 95%, or 100% of the length of the reference sequence. The nucleotides at corresponding nucleotide positions are then compared. When a position in the first sequence is occupied by the same nucleotide as the corresponding position in the second sequence, then the molecules are identical at that position. The percent identity between the two sequences is a function of the number of identical positions shared by the sequences, taking into account the number of gaps, and the length of each gap, which needs to be introduced for optimal alignment of the two sequences. The comparison of sequences and determination of percent identity between two sequences can be accomplished using a mathematical algorithm. When comparing DNA and RNA, thymine (T) and uracil (U) can be considered equivalent.

Suitable software programs are available from various sources and for alignment of both protein and nucleotide sequences. One suitable program to determine percent sequence identity is bl2seq, part of the BLAST suite of programs available from the U.S. government's National Center for Biotechnology Information BLAST website (blast.ncbi.nlm.nih.gov). Bl2seq performs a comparison between two sequences using either the BLASTN or BLASTP algorithm. BLASTN is used to compare nucleic acid sequences, while BLASTP is used to compare amino acid sequences. Other suitable programs are, e.g., Needle, Stretcher, Water, or Matcher, part of the EMBOSS suite of bioinformatics programs and also available from the European Bioinformatics Institute (EBI).

Sequence alignments can be conducted using methods known in the art such as MAFFT, Clustal (ClustalW, Clustal X or Clustal Omega), MUSCLE, etc.

Different regions within a single polynucleotide or polypeptide target sequence that aligns with a polynucleotide or polypeptide reference sequence can each have their own percent sequence identity.

In certain aspects, the percentage identity “% ID” of a first amino acid sequence (or nucleic acid sequence) to a second amino acid sequence (or nucleic acid sequence) is calculated as % ID=100×(Y/Z), where Y is the number of amino acid residues (or nucleobases) scored as identical matches in the alignment of the first and second sequences (as aligned by visual inspection or a particular sequence alignment program) and Z is the total number of residues in the second sequence. If the length of a first sequence is longer than the second sequence, the percent identity of the first sequence to the second sequence will be higher than the percent identity of the second sequence to the first sequence.

One skilled in the art will appreciate that the generation of a sequence alignment for the calculation of a percent sequence identity is not limited to binary sequence-sequence comparisons exclusively driven by primary sequence data. It will also be appreciated that sequence alignments can be generated by integrating sequence data with data from heterogeneous sources such as structural data (e.g., crystallographic protein structures), functional data (e.g., location of mutations), or phylogenetic data. A suitable program that integrates heterogeneous data to generate a multiple sequence alignment is T-Coffee, available at www.tcoffee.org, and alternatively available, e.g., from the EBI. It will also be appreciated that the final alignment used to calculate percent sequence identity can be curated either automatically or manually.

Immune response: The term “immune response” refers to the action of, for example, lymphocytes, antigen presenting cells, phagocytic cells, granulocytes, and soluble macromolecules produced by the above cells (including antibodies, cytokines, and complement) that results in selective damage to, destruction of, or elimination from the human body of invading pathogens, cells or tissues infected with pathogens, cancerous cells, or, in cases of autoimmunity or pathological inflammation, normal human cells or tissues.

Inflammatory response: “Inflammatory response” refers to immune responses involving specific and non-specific defense systems. A specific defense system reaction is a specific immune system reaction to an antigen. Examples of specific defense system reactions include antibody responses. A non-specific defense system reaction is an inflammatory response mediated by leukocytes generally incapable of immunological memory, e.g., macrophages, eosinophils, and neutrophils. In some aspects, an immune response includes the secretion of inflammatory cytokines, resulting in elevated inflammatory cytokine levels.

Inflammatory cytokines: The term “inflammatory cytokine” refers to cytokines that are elevated in an inflammatory response. Examples of inflammatory cytokines include interleukin-6 (IL-6), CXCL1 (chemokine (C-X-C motif) ligand 1; also known as GROc, interferon-γ (IFNγ), tumor necrosis factor α (TNFα), interferon γ-induced protein 10 (IP-10), or granulocyte-colony stimulating factor (G-CSF). The term inflammatory cytokines also include other cytokines associated with inflammatory responses known in the art, e.g., interleukin-1 (IL-1), interleukin-8 (IL-8), interleukin-12 (L-12), interleukin-13 (IL-13), interferon α (IFN-α), etc.

In Vitro: As used herein, the term “in vitro” refers to events that occur in an artificial environment, e.g., in a test tube or reaction vessel, in cell culture, in a Petri dish, etc., rather than within an organism (e.g., animal, plant, or microbe).

In Vivo: As used herein, the term “in vivo” refers to events that occur within an organism (e.g., animal, plant, or microbe or cell or tissue thereof).

Insertional and deletional variants: “Insertional variants” when referring to polypeptides are those with one or more amino acids inserted immediately adjacent to an amino acid at a particular position in a native or starting sequence. “Immediately adjacent” to an amino acid means connected to either the alpha-carboxy or alpha-amino functional group of the amino acid. “Deletional variants” when referring to polypeptides are those with one or more amino acids in the native or starting amino acid sequence removed. Ordinarily, deletional variants will have one or more amino acids deleted in a particular region of the molecule.

Intact: As used herein, in the context of a polypeptide, the term “intact” means retaining an amino acid corresponding to the wild type protein, e.g., not mutating or substituting the wild type amino acid. Conversely, in the context of a nucleic acid, the term “intact” means retaining a nucleobase corresponding to the wild type nucleic acid, e.g., not mutating or substituting the wild type nucleobase.

Isolated: As used herein, the term “isolated” refers to a substance or entity that has been separated from at least some of the components with which it was associated (whether in nature or in an experimental setting). Isolated substances (e.g., nucleotide sequence or protein sequence) can have varying levels of purity in reference to the substances from which they have been associated. Isolated substances and/or entities can be separated from at least about 10%, about 20%, about 30%, about 40%, about 50%, about 60%, about 70%, about 80%, about 90%, or more of the other components with which they were initially associated. In some aspects, isolated agents are more than about 80%, about 85%, about 90%, about 91%, about 92%, about 93%, about 94%, about 95%, about 96%, about 97%, about 98%, about 99%, or more than about 99% pure. As used herein, a substance is “pure” if it is substantially free of other components. The term “substantially isolated” means that the compound is substantially separated from the environment in which it was formed or detected. Partial separation can include, for example, a composition enriched in the compound of the present disclosure. Substantial separation can include compositions containing at least about 50%, at least about 60%, at least about 70%, at least about 80%, at least about 90%, at least about 95%, at least about 97%, or at least about 99% by weight of the compound of the present disclosure, or salt thereof.

A polynucleotide, vector, polypeptide, cell, or any composition disclosed herein which is “isolated” is a polynucleotide, vector, polypeptide, cell, or composition which is in a form not found in nature. Isolated polynucleotides, vectors, polypeptides, or compositions include those which have been purified to the degree that they are no longer in a form in which they are found in nature. In some aspects, a polynucleotide, vector, polypeptide, or composition which is isolated is substantially pure.

Isomer: As used herein, the term “isomer” means any tautomer, stereoisomer, enantiomer, or diastereomer of any compound of the disclosure. It is recognized that the compounds of the disclosure can have one or more chiral centers and/or double bonds and, therefore, exist as stereoisomers, such as double-bond isomers (i.e., geometric E/Z isomers) or diastereomers (e.g., enantiomers (i.e., (+) or (−)) or cis/trans isomers). According to the disclosure, the chemical structures depicted herein, and therefore the compounds of the disclosure, encompass all of the corresponding stereoisomers, that is, both the stereomerically pure form (e.g., geometrically pure, enantiomerically pure, or diastereomerically pure) and enantiomeric and stereoisomeric mixtures, e.g., racemates. Enantiomeric and stereoisomeric mixtures of compounds of the disclosure can typically be resolved into their component enantiomers or stereoisomers by well-known methods, such as chiral-phase gas chromatography, chiral-phase high performance liquid chromatography, crystallizing the compound as a chiral salt complex, or crystallizing the compound in a chiral solvent. Enantiomers and stereoisomers can also be obtained from stereomerically or enantiomerically pure intermediates, reagents, and catalysts by well-known asymmetric synthetic methods.

Linker: As used herein, a “linker” refers to a group of atoms, e.g., 10-1,000 atoms, and can be comprised of the atoms or groups such as, but not limited to, carbon, amino, alkylamino, oxygen, sulfur, sulfoxide, sulfonyl, carbonyl, and imine. The linker can be attached to a modified nucleoside or nucleotide on the nucleobase or sugar moiety at a first end, and to a payload, e.g., a detectable or therapeutic agent, at a second end. The linker can be of sufficient length as not to interfere with incorporation into a nucleic acid sequence. The linker can be used for any useful purpose, such as to form polynucleotide multimers (e.g., through linkage of two or more chimeric polynucleotides molecules or IVT polynucleotides) or polynucleotides conjugates, as well as to administer a payload, as described herein.

Monomers or multipers of polypeptides, e.g., amino acids or polynucleotides, e.g., nucleosides, may be utilized as linkers. For example, a short peptide may act as a linker between two proteins or polypeptides. Likewise, there may exist a series of nucleosides or nucleotides which serve as a linker between two polynucleotides.

Examples of chemical groups that can be incorporated into the linker include, but are not limited to, alkyl, alkenyl, alkynyl, amido, amino, ether, thioether, ester, alkylene, heteroalkylene, aryl, or heterocyclyl, each of which can be optionally substituted, as described herein. Examples of linkers include, but are not limited to, unsaturated alkanes, polyethylene glycols (e.g., ethylene or propylene glycol monomeric units, e.g., diethylene glycol, dipropylene glycol, triethylene glycol, tripropylene glycol, tetraethylene glycol, or tetraethylene glycol), and dextran polymers and derivatives thereof. Other examples include, but are not limited to, cleavable moieties within the linker, such as, for example, a disulfide bond (—S—S—) or an azo bond (—N═N—), which can be cleaved using a reducing agent or photolysis. Non-limiting examples of a selectively cleavable bond include an amido bond that can be cleaved, for example, by the use of tris(2-carboxyethyl)phosphine (TCEP), or other reducing agents, and/or photolysis, as well as an ester bond can be cleaved for example by acidic or basic hydrolysis.

Methods of Administration: As used herein, “methods of administration” may include intravenous, intramuscular, intradermal, subcutaneous, or other methods of delivering a composition to a subject. A method of administration may be selected to target delivery (e.g., to specifically deliver) to a specific region or system of a body.

Modified: As used herein, “modified” refers to a changed state or structure of a molecule of the disclosure. Molecules can be modified in many ways, including chemically, structurally, and functionally. In some aspects, the mRNA molecules of the present disclosure are modified by the introduction of non-natural nucleosides and/or nucleotides, e.g., as it relates to the natural ribonucleotides A, U, G, and C. Noncanonical nucleotides such as the cap structures are not considered “modified” although they differ from the chemical structure of the A, C, G, U ribonucleotides.

Naturally occurring: As used herein, “naturally occurring” means existing in nature without artificial aid.

Non-human vertebrate: As used herein, a “non-human vertebrate” includes all vertebrates except Homo sapiens, including wild and domesticated species. Examples of non-human vertebrates include, but are not limited to, mammals, such as alpaca, banteng, bison, camel, cat, cattle, deer, dog, donkey, gayal, goat, guinea pig, horse, llama, mule, pig, rabbit, reindeer, sheep water buffalo, and yak.

Nucleic acid sequence: The terms “nucleic acid sequence,” “nucleotide sequence,” or “polynucleotide sequence” are used interchangeably and refer to a contiguous nucleic acid sequence. The sequence can be either single stranded or double stranded DNA or RNA, e.g., an mRNA.

The term “nucleic acid,” in its broadest sense, includes any compound and/or substance that comprises a polymer of nucleotides. These polymers are often referred to as polynucleotides. Exemplary nucleic acids or polynucleotides of the disclosure include, but are not limited to, ribonucleic acids (RNAs), deoxyribonucleic acids (DNAs), threose nucleic acids (TNAs), glycol nucleic acids (GNAs), peptide nucleic acids (PNAs), locked nucleic acids (LNAs, including LNA having a β-D-ribo configuration, α-LNA having an α-L-ribo configuration (a diastereomer of LNA), 2′-amino-LNA having a 2′-amino functionalization, and 2′-amino-α-LNA having a 2′-amino functionalization), ethylene nucleic acids (ENA), cyclohexenyl nucleic acids (CeNA) or hybrids or combinations thereof.

The phrase “nucleotide sequence encoding” refers to the nucleic acid (e.g., an mRNA or DNA molecule) coding sequence which encodes a polypeptide. The coding sequence can further include initiation and termination signals operably linked to regulatory elements, including a promoter and polyadenylation signal capable of directing expression in the cells of an individual or mammal to which the nucleic acid is administered. The coding sequence can further include sequences that encode signal peptides.

Off-target: As used herein, “off-target” refers to any unintended effect on any one or more target, gene, or cellular transcript.

Open reading frame: As used herein, “open reading frame” or “ORF” refers to a sequence that does not contain a stop codon in a given reading frame.

Operably linked: As used herein, the phrase “operably linked” refers to a functional connection between two or more molecules, constructs, transcripts, entities, moieties, or the like.

Optionally substituted: Herein, a phrase of the form “optionally substituted X” (e.g., optionally substituted alkyl) is intended to be equivalent to “X, wherein X is optionally substituted” (e.g., “alkyl, wherein said alkyl is optionally substituted”). It is not intended to mean that the feature “X” (e.g., alkyl) per se is optional.

Part: As used herein, a “part” or “region” of a polynucleotide is defined as any portion of the polynucleotide that is less than the entire length of the polynucleotide Likewise, a “part” or “region” of a polypeptide is defined as any portion of the polypeptide that is less than the entire length of the polynucleotide.

Patient: As used herein, “patient” refers to a subject who may seek or be in need of treatment, requires treatment, is receiving treatment, will receive treatment, or a subject who is under care by a trained professional for a particular disease or condition.

Pharmaceutically acceptable: The phrase “pharmaceutically acceptable” is employed herein to refer to those compounds, materials, compositions, and/or dosage forms that are, within the scope of sound medical judgment, suitable for use in contact with the tissues of human beings and animals without excessive toxicity, irritation, allergic response, or other problem or complication, commensurate with a reasonable benefit/risk ratio.

Pharmaceutically acceptable excipients: The phrase “pharmaceutically acceptable excipient,” as used herein, refers to any ingredient other than the compounds described herein (for example, a vehicle capable of suspending or dissolving the active compound) and having the properties of being substantially nontoxic and non-inflammatory in a patient. Excipients can include, for example, antiadherents, antioxidants, binders, coatings, compression aids, disintegrants, dyes (colors), emollients, emulsifiers, fillers (diluents), film formers or coatings, flavors, fragrances, glidants (flow enhancers), lubricants, preservatives, printing inks, sorbents, suspending or dispersing agents, sweeteners, and waters of hydration. Exemplary excipients include, but are not limited to, butylated hydroxytoluene (BHT), calcium carbonate, calcium phosphate (dibasic), calcium stearate, croscarmellose, crosslinked polyvinyl pyrrolidone, citric acid, crospovidone, cysteine, ethylcellulose, gelatin, hydroxypropyl cellulose, hydroxypropyl methylcellulose, lactose, magnesium stearate, maltitol, mannitol, methionine, methylcellulose, methyl paraben, microcrystalline cellulose, polyethylene glycol, polyvinyl pyrrolidone, povidone, pregelatinized starch, propyl paraben, retinyl palmitate, shellac, silicon dioxide, sodium carboxymethyl cellulose, sodium citrate, sodium starch glycolate, sorbitol, starch (corn), stearic acid, sucrose, talc, titanium dioxide, vitamin A, vitamin E, vitamin C, and xylitol.

Pharmaceutically acceptable salts: The present disclosure also includes pharmaceutically acceptable salts of the compounds described herein. As used herein, “pharmaceutically acceptable salts” refers to derivatives of the disclosed compounds wherein the parent compound is modified by converting an existing acid or base moiety to its salt form (e.g., by reacting the free base group with a suitable organic acid). Examples of pharmaceutically acceptable salts include, but are not limited to, mineral or organic acid salts of basic residues such as amines; alkali or organic salts of acidic residues such as carboxylic acids; and the like. Representative acid addition salts include acetate, acetic acid, adipate, alginate, ascorbate, aspartate, benzenesulfonate, benzene sulfonic acid, benzoate, bisulfate, borate, butyrate, camphorate, camphorsulfonate, citrate, cyclopentanepropionate, digluconate, dodecylsulfate, ethanesulfonate, fumarate, glucoheptonate, glycerophosphate, hemisulfate, heptonate, hexanoate, hydrobromide, hydrochloride, hydroiodide, 2-hydroxy-ethanesulfonate, lactobionate, lactate, laurate, lauryl sulfate, malate, maleate, malonate, methanesulfonate, 2-naphthalenesulfonate, nicotinate, nitrate, oleate, oxalate, palmitate, pamoate, pectinate, persulfate, 3-phenylpropionate, phosphate, picrate, pivalate, propionate, stearate, succinate, sulfate, tartrate, thiocyanate, toluenesulfonate, undecanoate, valerate salts, and the like. Representative alkali or alkaline earth metal salts include sodium, lithium, potassium, calcium, magnesium, and the like, as well as nontoxic ammonium, quaternary ammonium, and amine cations, including, but not limited to ammonium, tetramethylammonium, tetraethylammonium, methylamine, dimethylamine, trimethylamine, triethylamine, ethylamine, and the like. The pharmaceutically acceptable salts of the present disclosure include the conventional non-toxic salts of the parent compound formed, for example, from non-toxic inorganic or organic acids. The pharmaceutically acceptable salts of the present disclosure can be synthesized from the parent compound that contains a basic or acidic moiety by conventional chemical methods. Generally, such salts can be prepared by reacting the free acid or base forms of these compounds with a stoichiometric amount of the appropriate base or acid in water or in an organic solvent, or in a mixture of the two; generally, nonaqueous media like ether, ethyl acetate, ethanol, isopropanol, or acetonitrile are used. Lists of suitable salts are found in Remington's Pharmaceutical Sciences, 17^{th} ed., Mack Publishing Company, Easton, Pa., 1985, p. 1418, Pharmaceutical Salts: Properties, Selection, and Use, P. H. Stahl and C. G. Wermuth (eds.), Wiley-VCH, 2008, and Berge et al., Journal of Pharmaceutical Science, 66, 1-19 (1977), each of which is incorporated herein by reference in its entirety.

Pharmaceutically acceptable solvate: The term “pharmaceutically acceptable solvate,” as used herein, means a compound of the disclosure wherein molecules of a suitable solvent are incorporated in the crystal lattice. A suitable solvent is physiologically tolerable at the dosage administered. For example, solvates can be prepared by crystallization, recrystallization, or precipitation from a solution that includes organic solvents, water, or a mixture thereof. Examples of suitable solvents are ethanol, water (for example, mono-, di-, and tri-hydrates), N-methylpyrrolidinone (NMP), dimethyl sulfoxide (DMSO), N,N′-dimethylformamide (DMF), N,N′-dimethylacetamide (DMAC), 1,3-dimethyl-2-imidazolidinone (DMEU), 1,3-dimethyl-3,4,5,6-tetrahydro-2-(1H)-pyrimidinone (DMPU), acetonitrile (ACN), propylene glycol, ethyl acetate, benzyl alcohol, 2-pyrrolidone, benzyl benzoate, and the like. When water is the solvent, the solvate is referred to as a “hydrate.”

Pharmacokinetic: As used herein, “pharmacokinetic” refers to any one or more properties of a molecule or compound as it relates to the determination of the fate of substances administered to a living organism. Pharmacokinetics is divided into several areas, including the extent and rate of absorption, distribution, metabolism, and excretion. This is commonly referred to as ADME where: (A) Absorption is the process of a substance entering the blood circulation; (D) Distribution is the dispersion or dissemination of substances throughout the fluids and tissues of the body; (M) Metabolism (or Biotransformation) is the irreversible transformation of parent compounds into daughter metabolites; and (E) Excretion (or Elimination) refers to the elimination of the substances from the body. In rare cases, some drugs irreversibly accumulate in body tissue.

Physicochemical. As used herein, “physicochemical” means of or relating to a physical and/or chemical property.

Polynucleotide: The term “polynucleotide” as used herein refers to polymers of nucleotides of any length, including ribonucleotides, deoxyribonucleotides, analogs thereof, or mixtures thereof. This term refers to the primary structure of the molecule. Thus, the term includes triple-, double- and single-stranded deoxyribonucleic acid (“DNA”), as well as triple-, double- and single-stranded ribonucleic acid (“RNA”). It also includes modified, for example by alkylation, and/or by capping, and unmodified forms of the polynucleotide. More particularly, the term “polynucleotide” includes polydeoxyribonucleotides (containing 2-deoxy-D-ribose), polyribonucleotides (containing D-ribose), including tRNA, rRNA, hRNA, siRNA, and mRNA, whether spliced or unspliced, any other type of polynucleotide which is an N- or C-glycoside of a purine or pyrimidine base, and other polymers containing normucleotidic backbones, for example, polyamide (e.g., peptide nucleic acids “PNAs”) and polymorpholino polymers, and other synthetic sequence-specific nucleic acid polymers providing that the polymers contain nucleobases in a configuration which allows for base pairing and base stacking, such as is found in DNA and RNA. In particular aspects, the polynucleotide comprises an mRNA. In other aspects, the mRNA is a synthetic mRNA. In some aspects, the synthetic mRNA comprises at least one unnatural nucleobase. In some aspects, all nucleobases of a certain class have been replaced with unnatural nucleobases (e.g., all uridines in a polynucleotide disclosed herein can be replaced with an unnatural nucleobase, e.g., 5-methoxyuridine). In some aspects, the polynucleotide (e.g., a synthetic RNA or a synthetic DNA) comprises only natural nucleobases, i.e., A,C, T, and U in the case of a synthetic DNA, or A, C, T, and U in the case of a synthetic RNA.

The skilled artisan will appreciate that the T bases in the codon maps disclosed herein are present in DNA, whereas the T bases would be replaced by U bases in corresponding RNAs. For example, a codon-nucleotide sequence disclosed herein in DNA form, e.g., a vector or an in-vitro translation (IVT) template, would have its T bases transcribed as U based in its corresponding transcribed mRNA. In this respect, both codon-optimized DNA sequences (comprising T) and their corresponding RNA sequences (comprising U) are considered codon-optimized nucleotide sequences of the present disclosure. A skilled artisan would also understand that equivalent codon-maps can be generated by replaced one or more bases with non-natural bases. Thus, e.g., a TTC codon (DNA map) would correspond to a UUC codon (RNA map), which in turn would correspond to a ‘P’C codon (RNA map in which U has been replaced with pseudouridine).

Standard A-T and G-C base pairs form under conditions that allow the formation of hydrogen bonds between the N3-H and C4-oxy of thymidine and the N1 and C6-NH₂, respectively, of adenosine and between the C2-oxy, N3, and C4-NH₂, of cytidine and the C2-NH₂, N′—H and C6-oxy, respectively, of guanosine. Thus, for example, guanosine (2-amino-6-oxy-9-β-D-ribofuranosyl-purine) can be modified to form isoguanosine (2-oxy-6-amino-9-β-D-ribofuranosyl-purine). Such modification results in a nucleoside base, which will no longer effectively form a standard base pair with cytosine. However, modification of cytosine (1-β-D-ribofuranosyl-2-oxy-4-amino-pyrimidine) to form isocytosine (1-β-D-ribofuranosyl-2-amino-4-oxy-pyrimidine-) results in a modified nucleotide which will not effectively base pair with guanosine but will form a base pair with isoguanosine (U.S. Pat. No. 5,681,702 to Collins et al.). Isocytosine is available from Sigma Chemical Co. (St. Louis, Mo.); isocytidine can be prepared by the method described by Switzer et al. (1993) Biochemistry 32:10489-10496 and references cited therein; 2′-deoxy-5-methyl-isocytidine can be prepared by the method of Tor et al. (1993) J. Am. Chem. Soc. 115:4461-4467, and references cited therein; and isoguanine nucleotides can be prepared using the method described by Switzer et al., 1993, supra, and Mantsch et al. (1993) Biochem. 14:5593-5601, or by the method described in U.S. Pat. No. 5,780,610 to Collins et al. Other nonnatural base pairs can be synthesized by the method described in Piccirilli et al. (1990) Nature 343:33-37, for the synthesis of 2,6-diaminopyrimidine and its complement (1-methylpyrazolo-[4,3]pyrimidine-5,7-(4H,6H)-dione. Other such modified nucleotide units which form unique base pairs are known, such as those described in Leach et al. (1992) J. Am. Chem. Soc. 114:3675-3683 and Switzer et al., supra.

Nucleic acid sequence: The terms “nucleic acid sequence,” “nucleotide sequence,” or “polynucleotide” are used interchangeably and refer to a contiguous nucleic acid sequence. The sequence can be either single stranded or double stranded DNA or RNA, e.g., an mRNA.

The phrase “nucleotide sequence encoding” and variants thereof refers to the nucleic acid (e.g., an mRNA or DNA molecule) coding sequence that comprises a nucleotide sequence that encodes a polypeptide or functional fragment thereof as set forth herein. The coding sequence can further include initiation and termination signals operably linked to regulatory elements, including a promoter and polyadenylation signal capable of directing expression in the cells of an individual or mammal to which the nucleic acid is administered. The coding sequence can further include sequences that encode signal peptides.

Polypeptide: The terms “polypeptide,” “peptide,” and “protein” are used interchangeably herein to refer to polymers of amino acids of any length. The polymer can comprise modified amino acids. The terms also encompass an amino acid polymer that has been modified naturally or by intervention; for example, disulfide bond formation, glycosylation, lipidation, acetylation, phosphorylation, or any other manipulation or modification, such as conjugation with a labeling component. Also included within the definition are, for example, polypeptides containing one or more analogs of an amino acid (including, for example, unnatural amino acids such as homocysteine, ornithine, p-acetylphenylalanine, D-amino acids, and creatine), as well as other modifications known in the art.

The term, as used herein, refers to proteins, polypeptides, and peptides of any size, structure, or function. Polypeptides include gene products, naturally occurring polypeptides, synthetic polypeptides, homologs, orthologs, paralogs, fragments and other equivalents, variants, and analogs of the foregoing. A polypeptide can be a single polypeptide or can be a multi-molecular complex such as a dimer, trimer, or tetramer. They can also comprise single chain or multichain polypeptides. Most commonly, disulfide linkages are found in multichain polypeptides. The term polypeptide can also apply to amino acid polymers in which one or more amino acid residues are an artificial chemical analogue of a corresponding naturally occurring amino acid. In some aspects, a “peptide” can be less than or equal to 50 amino acids long, e.g., about 5, 10, 15, 20, 25, 30, 35, 40, 45, or 50 amino acids long.

Polypeptide variant: As used herein, the term “polypeptide variant” refers to molecules that differ in their amino acid sequence from a native or reference sequence. The amino acid sequence variants can possess substitutions, deletions, and/or insertions at certain positions within the amino acid sequence, as compared to a native or reference sequence. Ordinarily, variants will possess at least about 50% identity, at least about 60% identity, at least about 70% identity, at least about 80% identity, at least about 90% identity, at least about 95% identity, at least about 99% identity to a native or reference sequence. In some aspects, they will be at least about 80%, or at least about 90% identical to a native or reference sequence.

Preventing: As used herein, the term “preventing” refers to partially or completely delaying the onset of an infection, disease, disorder and/or condition; partially or completely delaying the onset of one or more symptoms, features, or clinical manifestations of a particular infection, disease, disorder, and/or condition; partially or completely delaying the onset of one or more symptoms, features, or manifestations of a particular infection, disease, disorder, and/or condition; partially or completely delaying progression from an infection, a particular disease, disorder and/or condition; and/or decreasing the risk of developing pathology associated with the infection, the disease, disorder, and/or condition.

Prophylactic: As used herein, “prophylactic” refers to a therapeutic or course of action used to prevent the spread of disease.

Prophylaxis: As used herein, a “prophylaxis” refers to a measure taken to maintain health and prevent the spread of disease. An “immune prophylaxis”, e.g., a vaccine, refers to a measure to produce active or passive immunity to prevent the spread of disease.

Protein cleavage site: As used herein, “protein cleavage site” refers to a site where controlled cleavage of the amino acid chain can be accomplished by chemical, enzymatic or photochemical means.

Protein cleavage signal: As used herein, “protein cleavage signal” refers to at least one amino acid that flags or marks a polypeptide for cleavage.

Proteins of interest: As used herein, the terms “proteins of interest” or “desired proteins” include those provided herein and fragments, mutants, variants, and alterations thereof.

Proximal: As used herein, the term “proximal” means situated nearer to the center or to a point or region of interest.

Pseudouridine: As used herein, pseudouridine refers to the C-glycoside isomer of the nucleoside uridine. A “pseudouridine analog” is any modification, variant, isoform or derivative of pseudouridine. For example, pseudouridine analogs include but are not limited to 1-carboxymethyl-pseudouridine, 1-propynyl-pseudouridine, 1-taurinomethyl-pseudouridine, 1-taurinomethyl-4-thio-pseudouridine, 1-methylpseudouridine (m¹ψ), 1-methyl-4-thio-pseudouridine), 4-thio-1-methyl-pseudouridine, 3-methyl-pseudouridine (m³ψ), 2-thio-1-methyl-pseudouridine, 1-methyl-1-deaza-pseudouridine, 2-thio-1-methyl-1-deaza-pseudouridine, dihydropseudouridine, 2-thio-dihydropseudouridine, 2-methoxyuridine, 2-methoxy-4-thio-uridine, 4-methoxy-pseudouridine, 4-methoxy-2-thio-pseudouridine, N1-methyl-pseudouridine, 1-methyl-3-(3-amino-3-carboxypropyl)pseudouridine (acp³ψ), and 2′-O-methyl-pseudouridine (xm).

Purified: As used herein, “purify,” “purified,” “purification” means to make substantially pure or clear from unwanted components, material defilement, admixture, or imperfection.

Reference Nucleic Acid Sequence: The term “reference nucleic acid sequence” or “reference nucleic acid” or “reference nucleotide sequence” or “reference sequence” refers to a starting nucleic acid sequence (e.g., a RNA, e.g., an mRNA sequence) that can be sequence optimized. In some aspects, the reference nucleic acid sequence is a wild type nucleic acid sequence, a fragment or a variant thereof. In some aspects, the reference nucleic acid sequence is a previously sequence optimized nucleic acid sequence.

Salts: In some aspects, the pharmaceutical or therapeutic composition for intratumoral delivery is disclosed herein and comprises salts of some of their lipid constituents. The term “salt” includes any anionic and cationic complex. Non-limiting examples of anions include inorganic and organic anions, e.g., fluoride, chloride, bromide, iodide, oxalate (e.g., hemioxalate), phosphate, phosphonate, hydrogen phosphate, dihydrogen phosphate, oxide, carbonate, bicarbonate, nitrate, nitrite, nitride, bisulfite, sulfide, sulfite, bisulfate, sulfate, thiosulfate, hydrogen sulfate, borate, formate, acetate, benzoate, citrate, tartrate, lactate, acrylate, polyacrylate, fumarate, maleate, itaconate, glycolate, gluconate, malate, mandelate, tiglate, ascorbate, salicylate, polymethacrylate, perchlorate, chlorate, chlorite, hypochlorite, bromate, hypobromite, iodate, an alkylsulfonate, an arylsulfonate, arsenate, arsenite, chromate, dichromate, cyanide, cyanate, thiocyanate, hydroxide, peroxide, permanganate, and mixtures thereof.

Sample: As used herein, the term “sample” or “biological sample” refers to a subset of its tissues, cells, or component parts (e.g., body fluids, including but not limited to blood, mucus, lymphatic fluid, synovial fluid, cerebrospinal fluid, saliva, amniotic fluid, amniotic cord blood, urine, vaginal fluid, and semen). A sample further can include a homogenate, lysate or extract prepared from a whole organism or a subset of its tissues, cells or component parts, or a fraction or portion thereof, including but not limited to, for example, plasma, serum, spinal fluid, lymph fluid, the external sections of the skin, respiratory, intestinal, and genitourinary tracts, tears, saliva, milk, blood cells, tumors, organs. A sample further refers to a medium, such as a nutrient broth or gel, which may contain cellular components, such as proteins or nucleic acid molecules.

Side Scatter (SSC): Side scatter, or SSC is a flow cytometry measurement that measures the light scattered by cells at a ninety-degree angle relative to the laser.

Signal Sequence: As used herein, the phrases “signal sequence,” “signal peptide,” and “transit peptide” are used interchangeably and refer to a sequence that can direct the transport or localization of a protein to a certain organelle, cell compartment, or extracellular export. The term encompasses both the signal sequence polypeptide and the nucleic acid sequence encoding the signal sequence. Thus, references to a signal sequence in the context of a nucleic acid refer in fact to the nucleic acid sequence encoding the signal sequence polypeptide.

Similarity: As used herein, the term “similarity” refers to the overall relatedness between polymeric molecules, e.g., between polynucleotide molecules (e.g., DNA molecules and/or RNA molecules) and/or between polypeptide molecules. Calculation of percent similarity of polymeric molecules to one another can be performed in the same manner as a calculation of percent identity, except that calculation of percent similarity takes into account conservative substitutions as is understood in the art.

Specific delivery: As used herein, the term “specific delivery,” “specifically deliver,” or “specifically delivering” means delivery of more (e.g., at least 1.5 fold more, at least 2-fold more, at least 3-fold more, at least 4-fold more, at least 5-fold more, at least 6-fold more, at least 7-fold more, at least 8-fold more, at least 9-fold more, at least 10-fold more) of a polynucleotide to a target tissue of interest (e.g., mammalian liver) compared to an off-target tissue (e.g., mammalian spleen). The level of delivery to a particular tissue may be measured by comparing the amount of protein produced in a tissue to the weight of said tissue, comparing the amount of polynucleotide in a tissue to the weight of said tissue, comparing the amount of protein produced in a tissue to the amount of total protein in said tissue, or comparing the amount of polynucleotide in a tissue to the amount of total polynucleotide in said tissue.

Stable: As used herein, “stable” refers to a compound that is sufficiently robust to survive isolation to a useful degree of purity from a reaction mixture, and in some cases capable of formulation into an efficacious therapeutic agent.

Stabilized: As used herein, the term “stabilize,” “stabilized,” “stabilized region” means to make or become stable.

Stereoisomer: As used herein, the term “stereoisomer” refers to all possible different isomeric as well as conformational forms that a compound may possess (e.g., a compound of any formula described herein), in particular, all possible stereochemically and conformationally isomeric forms, all diastereomers, enantiomers and/or conformers of the basic molecular structure. Some compounds of the present disclosure may exist in different tautomeric forms, all of the latter being included within the scope of the present disclosure.

Subject: By “subject” or “individual” or “animal” or “patient” or “mammal,” is meant any subject, particularly a mammalian subject, for whom diagnosis, prognosis, or therapy is desired. Mammalian subjects include, but are not limited to, humans, domestic animals, farm animals, zoo animals, sport animals, pet animals such as dogs, cats, guinea pigs, rabbits, rats, mice, horses, cattle, cows; primates such as apes, monkeys, orangutans, and chimpanzees; canids such as dogs and wolves; felids such as cats, lions, and tigers; equids such as horses, donkeys, and zebras; bears, food animals such as cows, pigs, and sheep; ungulates such as deer and giraffes; rodents such as mice, rats, hamsters, and guinea pigs; and so on. In certain aspects, the mammal is a human subject. In other aspects, a subject is a human patient.

Substantially: As used herein, the term “substantially” refers to the qualitative condition of exhibiting total or near-total extent or degree of a characteristic or property of interest. One of ordinary skill in the biological arts will understand that biological and chemical phenomena rarely, if ever, go to completion and/or proceed to completeness or achieve or avoid an absolute result. The term “substantially” is therefore used herein to capture the potential lack of completeness inherent in many biological and chemical phenomena.

Substantially equal: As used herein as it relates to time differences between doses, the term means plus/minus 2%.

Substantially simultaneous: As used herein and as it relates to a plurality of doses, the term means within a few (e.g., 2) seconds.

Suffering from: An individual who is “suffering from” a disease, disorder, and/or condition has been diagnosed with or displays one or more symptoms of the disease, disorder, and/or condition.

Susceptible to: An individual who is “susceptible to” a disease, disorder, and/or condition has not been diagnosed with and/or may not exhibit symptoms of the disease, disorder, and/or condition but harbors a propensity to develop a disease or its symptoms. In some aspects, an individual who is susceptible to a disease, disorder, and/or condition (for example, cancer) can be characterized by one or more of the following: (1) a genetic mutation associated with the development of the disease, disorder, and/or condition; (2) a genetic polymorphism associated with the development of the disease, disorder, and/or condition; (3) increased and/or decreased expression and/or activity of a protein and/or nucleic acid associated with the disease, disorder, and/or condition; (4) habits and/or lifestyles associated with the development of the disease, disorder, and/or condition; (5) a family history of the disease, disorder, and/or condition; and (6) exposure to and/or infection with a microbe associated with the development of the disease, disorder, and/or condition. In some aspects, an individual who is susceptible to a disease, disorder, and/or condition will develop the disease, disorder, and/or condition. In some aspects, an individual who is susceptible to a disease, disorder, and/or condition will not develop the disease, disorder, and/or condition.

Sustained release: As used herein, the term “sustained release” refers to a pharmaceutical or therapeutic composition or compound release profile that conforms to a release rate over a specific period of time.

Synthetic: The term “synthetic” means produced, prepared, and/or manufactured by the hand of man. Synthesis of polynucleotides or other molecules of the present disclosure can be chemical or enzymatic.

Targeted cells: As used herein, “targeted cells” refers to any one or more cells of interest. The cells may be found in vitro, in vivo, in situ, or in the tissue or organ of an organism. The organism may be an animal, preferably a mammal, more preferably a human, and most preferably a patient.

Target tissue: As used herein, “target tissue” refers to any one or more tissue types of interest in which the delivery of a polynucleotide would result in a desired biological and/or pharmacological effect. Examples of target tissues of interest include specific tissues, organs, and systems or groups thereof. An “off-target tissue” refers to any one or more tissue types in which the expression of the encoded protein does not result in a desired biological and/or pharmacological effect.

Targeting sequence: As used herein, the phrase “targeting sequence” refers to a sequence that can direct the transport or localization of a protein or polypeptide.

Terminus: As used herein, the terms “termini” or “terminus,” when referring to polypeptides, refers to an extremity of a peptide or polypeptide. Such extremity is not limited only to the first or final site of the peptide or polypeptide but can include additional amino acids in the terminal regions. The polypeptide based molecules of the disclosure can be characterized as having both an N-terminus (terminated by an amino acid with a free amino group (NH₂)) and a C-terminus (terminated by an amino acid with a free carboxyl group (COOH)). Proteins of the disclosure are in some cases made up of multiple polypeptide chains brought together by disulfide bonds or by non-covalent forces (multimers, oligomers). These sorts of proteins will have multiple N- and C-termini. Alternatively, the termini of the polypeptides can be modified such that they begin or end, as the case can be, with a non-polypeptide-based moiety such as an organic conjugate.

Therapeutic Agent: The term “therapeutic agent” refers to an agent that, when administered to a subject, has a therapeutic, diagnostic, and/or prophylactic effect and/or elicits a desired biological and/or pharmacological effect.

Therapeutically effective amount: As used herein, the term “therapeutically effective amount” means an amount of an agent to be delivered (e.g., nucleic acid, drug, therapeutic agent, diagnostic agent, prophylactic agent, etc.) that is sufficient, when administered to a subject suffering from or susceptible to an infection, disease, disorder, and/or condition, to treat, improve symptoms of, diagnose, prevent, and/or delay the onset of the infection, disease, disorder, and/or condition.

Therapeutically effective outcome: As used herein, the term “therapeutically effective outcome” means an outcome that is sufficient in a subject suffering from or susceptible to an infection, disease, disorder, and/or condition, to treat, improve symptoms of, diagnose, prevent, and/or delay the onset of the infection, disease, disorder, and/or condition.

Transcription: As used herein, the term “transcription” refers to methods to introduce exogenous nucleic acids into a cell. Methods of transfection include, but are not limited to, chemical methods, physical treatments, and cationic lipids or mixtures.

Transfection: As used herein, “transfection” refers to the introduction of a polynucleotide into a cell wherein a polypeptide encoded by the polynucleotide is expressed (e.g., mRNA) or the polypeptide modulates a cellular function (e.g., siRNA, miRNA). As used herein, “expression” of a nucleic acid sequence refers to the translation of a polynucleotide (e.g., an mRNA) into a polypeptide or protein and/or post-translational modification of a polypeptide or protein.

Treating, treatment, therapy: As used herein, the term “treating” or “treatment” or “therapy” refers to partially or completely alleviating, ameliorating, improving, relieving, delaying the onset of, inhibiting the progression of, reducing the severity of, and/or reducing the incidence of one or more symptoms or features of a hyper-proliferative disease, e.g., cancer. For example, “treating” cancer can refer to inhibiting survival, growth, and/or spread of a tumor. Treatment can be administered to a subject who does not exhibit signs of a disease, disorder, and/or condition and/or to a subject who exhibits only early signs of a disease, disorder, and/or condition for the purpose of decreasing the risk of developing pathology associated with the disease, disorder, and/or condition.

Unmodified: As used herein, “unmodified” refers to any substance, compound or molecule prior to being changed in any way. Unmodified can, but does not always, refer to the wild type or native form of a biomolecule. Molecules can undergo a series of modifications whereby each modified molecule can serve as the “unmodified” starting molecule for a subsequent modification.

Variant: The term variant as used in the present disclosure refers to both natural variants (e.g., polymorphisms, isoforms, etc.) and artificial variants in which at least one amino acid residue in a native or starting sequence (e.g., a wild type sequence) has been removed and a different amino acid inserted in its place at the same position. These variants can be described as “substitutional variants.” The substitutions can be single, where only one amino acid in the molecule has been substituted, or they can be multiple, where two or more amino acids have been substituted in the same molecule. If amino acids are inserted or deleted, the resulting variant would be an “insertional variant” or a “deletional variant”, respectively.

The details of one or more aspects of the disclosure are set forth in the accompanying description below. Although any materials and methods similar or equivalent to those described herein can be used in the practice or testing of the present disclosure, the preferred materials and methods are now described. Other features, objects, and advantages of the disclosure will be apparent from the description. In the description, the singular forms also include the plural unless the context clearly dictates otherwise. Unless defined otherwise, all technical and scientific terms used herein have the same meaning as commonly understood by one of ordinary skill in the art to which this disclosure belongs. In the case of conflict, the present description will control.

The present disclosure is further illustrated by the following non-limiting examples.

### EXAMPLES

### Example 1. Flow Cytometric Analysis of SIINFEKL-MHCI Presentation

Murine dendritic cells (JAWSII) were seeded 100,000 cells/well in a 24 well plate (500 μL volume) and treated with a final dose of 200 ng mRNA (encoding CD1 vaccine cassettes including murine CD1d, human CD1d, and human CD1b) per formulation per well.

The mRNA vaccine comprising the murine CD1d cassette has the sequence:

ctagcGAGAGAAAAGAAGAGUAAGAAGAAAUAUAAGAGCCACCAUGCGCU
ACCUGCCUUGGCUGCUGCUGUGGGCUUUUCUGCAAGUGUGGGGCCAGUCU
GAGGCCCUGGAAUCCAUCAUCAACUUCGAGAAGCUGACCGAGCUGAUCGU
GUUCAUCGUGCUGAUCAUGCUGGUGGUCGUGGGCGCCGUGGUGUACUACA
UUUGGAGAAGAAGAAGCGCCUACCAGGACAUCAGAUGAGUUAAUUAAGCU
GCCUUCUGCGGGGCUUGCCUUCUGGCCAUGCCCUUCUUCUCUCCCUUGCA
CCUGUACCUCUUGGUCUUUGAAUAAAGCCUGAGUAGGAAGcccgggcgga
ttAAAAAAAAAAAAAAAAAAAAAAAAAAAAAAAAAAAAAAAAAAAAAAAA
AAAAAAAAAAAAAAAAAAAAAAAAAAAAAAAAAAAAAAAAAAAAAAAAAA
AAAAAAAAAAAAAAAAAAAAAAAAAAAAAAAAAAAAAAAAAAAAAAAAAA
AAAAAAAAAAAAAAAAAAAAAAAAAAAAAAAAAAAAAAAAAAAAAAAAAA
AAAAAAAAAAAAAAAAAAAAAAAAAAAAAAAAAAAAAAAAAAAAAAAAAA
AAAAAAAAAAAAAAAAAAAAAAAAAAAAAAAAAAAAAAAAAAAAAAAAAA
AAAAAAAAAAAAAAAAAAAAAAAAAAAAAAAAAAAAAAAAAAAAAAAAAA
AAAAAAAAAA.

The mRNA vaccine comprising the human CD1d cassette has the sequence:

ctagcGAGAGAAAAGAAGAGUAAGAAGAAAUAUAAGAGCCACCAUGGGCU
GCCUGCUGUUUCUGCUGCUUUGGGCUCUGCUGCAGGCCUGGGGAUCUGCC
CUGGAAUCCAUCAUCAACUUCGAGAAGCUGACCGAGAUGGGCCUGAUCGC
UCUGGCUGUUCUGGCCUGUCUGCUGUUCCUCCUGAUCGUGGGCUUCACCA
GCAGAUUCAAGAGACAGACCAGCUACCAGGGCGUGCUCUGAGUUAAUUAA
GCUGCCUUCUGCGGGGCUUGCCUUCUGGCCAUGCCCUUCUUCUCUCCCUU
GCACCUGUACCUCUUGGUCUUUGAAUAAAGCCUGAGUAGGAAGcccgggc
ggattAAAAAAAAAAAAAAAAAAAAAAAAAAAAAAAAAAAAAAAAAAAAA
AAAAAAAAAAAAAAAAAAAAAAAAAAAAAAAAAAAAAAAAAAAAAAAAAA
AAAAAAAAAAAAAAAAAAAAAAAAAAAAAAAAAAAAAAAAAAAAAAAAAA
AAAAAAAAAAAAAAAAAAAAAAAAAAAAAAAAAAAAAAAAAAAAAAAAAA
AAAAAAAAAAAAAAAAA.

The mRNA vaccine comprising the human CD1b cassette has the sequence:

ctagcGAGAGAAAAGAAGAGUAAGAAGAAAUAUAAGAGCCACCAUGCUGC
UGCUGCCCUUCCAGCUGCUGGCUGUUCUUUUUCCUGGCGGCAACCUGGAA
UCCAUCAUCAACUUCGAGAAGCUGACCGAGAUCGUGCUGGCCAUCAUCGU
GCCUUCUCUGCUGCUCCUGCUGUGUCUGGCCCUGUGGUACAUGAGAAGAA
GAAGCUACCAGAACAUCCCCUGAGUUAAUUAAGCUGCCUUCUGCGGGGCU
UGCCUUCUGGCCAUGCCCUUCUUCUCUCCCUUGCACCUGUACCUCUUGGU
CUUUGAAUAAAGCCUGAGUAGGAAGcccgggcggattAAAAAAAAAAAAA
AAAAAAAAAAAAAAAAAAAAAAAAAAAAAAAAAAAAAAAAAAAAAAAAAA
AAAAAAAAAAAAAAAAAAAAAAAAAAAAAAAAAAAAAAAAAAAAAAAAAA
AAAAAAAAAAAAAAAAAAAAAAAAAAAAAAAAAAAAAAAAAAAAAAAAAA
AAAAAAAAAAAAAAAAAAAAAAAAAAAAAAAAAAAAAAAAAAAAAAAAAA
AAAAAAAAAAAAAAAAAAA.

Included in each of the CD1 scaffolds is a stuffer sequence (ctagc) just after the T7 promoter sequence.

Cells were incubated at 37° C./5% CO₂ overnight. After incubation, cells were aliquoted into a 96-well plate (approximately 250 μL), and cells were blocked with Fc-block (100 μL/sample) and washed with FACS buffer (dPBS pH 7.5 with 5% Fetal Bovine Serum) before staining with PE-Cy5 conjugated anti-murine CD11c antibody and PE conjugated anti-mouse H-2 kb bound to antigen (SIINFEKL). In addition, each sample was stained with Zombie Near-infrared stain for discriminating dead cells from live cells.

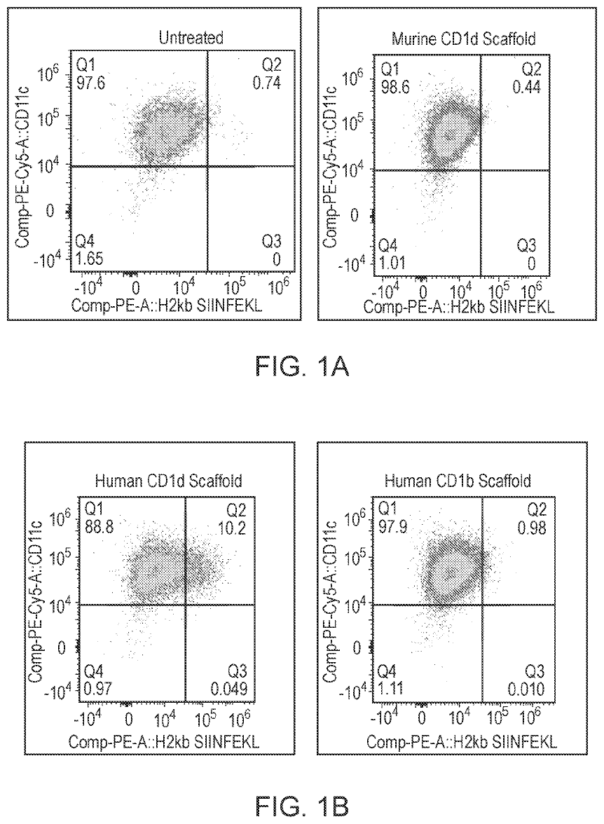

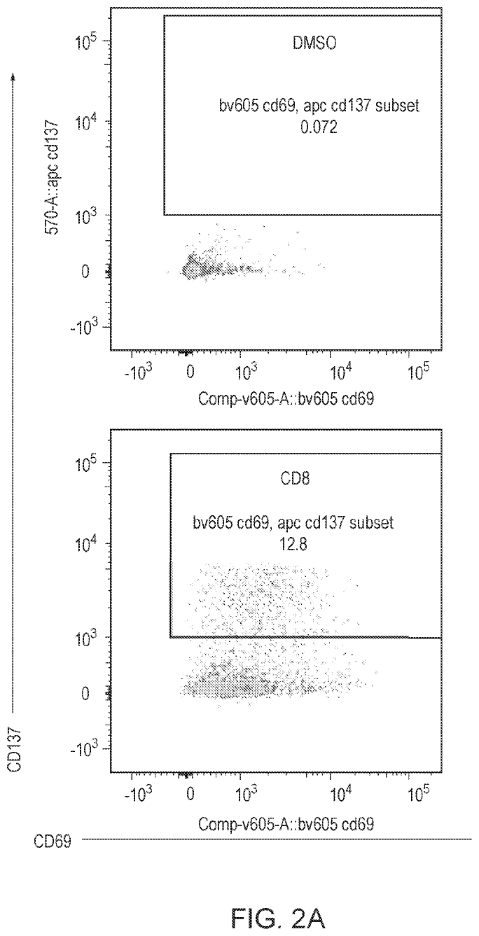

These data, comparing antigen presentation in the JAWS dendritic cell model for an epitope in the context of different scaffolds, revealed that the hCD1d scaffold had the best performance with 10.2% of antigen presenting cells showing specific epitope presentation as measured by flow cytometry compared to untreated sample. See FIG. 1.

### Example 2. In Vivo Studies

In in vivo studies, vaccines described herein are evaluated against commercially available materials. In this study, mRNA vaccines are evaluated for levels of SIINFEKL presentation on MHC-I compared to the commercial control. Reproducibility is likewise demonstrated, with multiple batches) resulting in similar levels of SIINFEKL+JAWSII cells.

In this study, mRNA vaccines are evaluated as vaccine candidates in a murine in vivo experiment. C57BL/6 mice are injected (IV) with commercial or mRNA vaccines described herein formulated in a delivery vehicle. Seven days post-injection, peripheral blood is isolated and stained using a fluorescent MHC-I tetramer specific for T-cells recognizing the OVA epitope. The fraction of OVA-specific CD8+ T-cells are then quantified by flow cytometry. In this experiment, mRNA vaccines are expected to result in an increase in the fraction of OVA-specific T-cells in peripheral blood relative to the commercial control, indicating the strength of these molecules as vaccines.

### Example 3. Ex-Vivo Stimulation in Healthy Donor: Pp65

### Example 3A

Cryopreserved Human Cytomegaly Virus (CMV) sero-positive Healthy Donor (CMV+) Peripheral blood mononuclear cells were thawed and resuspended in 14 mL RPMI1640. Cells were pelleted by centrifugation at 1200 rpm for 10 minutes. Supernatant was aspired and cells were re-suspended in and counted in appropriate volume of culture media (1:1 AIM-V/RPMI 1640+10% filtered human AB Serum+50 μM B-mercaptoethanol (TC grade)). Cells were rested overnight at 37 degrees Celsius in CO₂ incubator (5% CO₂).

After incubation, cells were treated with 50 ng mRNA encoding native CMV pp65 protein, 50 ng mRNA encoding pp65 with MHC presentation enhancing sequences, 2 ug/mL CMV pp65 peptide pool covering the entire pp65 molecule, or non-coding mRNA. Cells were incubated at 37 degrees Celsius in CO₂ incubator (5% CO₂) for 24 hrs.

After 24 hrs cells were harvested and washed twice in phosphate buffered saline (PBS) pH 7.2. Washed cells were then stained with Zombie Near Infrared live dead stain (NIR)(BioLegend) in PBS for 15 minutes at room temperature (RT).

Cells were then washed and re-suspended in 100 ul FACS buffer (PBS+0.5% BSA+0.02% Sodium Azid) containing fluorochrome conjugated a-CD8, a-CD4, a-CD137, and a-CD69 (BioLegend). Cells were then incubated for 20 minutes at room temperature. After staining, cells were washed twice with 200 ul PBS followed by 10 minutes centrifugation at 1200 rpm. After the final wash, the supernatant was discarded, and cells were resuspended in 200 ul PBS. Resuspended cells were then analyzed on a flow cytometer (Cytek).

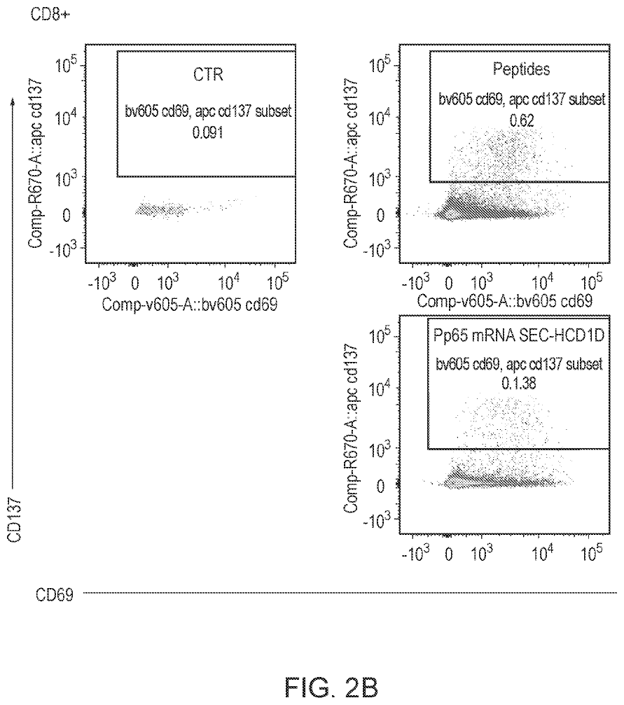

Results: Percentage of activated cells are indicated by in black square in FIGS. 2A and 2B. The control groups include DMSO (negative control), CD3 (positive control), and CTR (cell treated with non-coding mRNA nanoparticles). The treatment groups include Peptides (cells treated with 2 uM of CMV pp65 peptide pool covering the entire pp65 protein in overlapping sequences) and Pp65 Sec-hCD1d (Cells treated with Sec-pp65-hCD1d mRNA nanoparticles).

Observation: Compared to controls and peptide treated cells, Sec-pp65-hCD1d mRNA nanoparticle treated cells showed two-times more activated cells. This indicates that treatment of cells with hCD1d enhanced mRNA encoding for the entire pp65 protein enables effective antigen processing and presentation. This enhancement results in better and broader T cell activation.

### Example 3B

Cryopreserved Human Cytomegaly Virus (CMV) sero positive Healthy Donor (CMV+) Peripheral blood mononuclear cells were thawed and resuspended in 14 mL RPMI1640. Cells were pelleted by centrifugation at 1200 rpm for 10 minutes. Supernatant was aspired and cells were re-suspended in and counted in appropriate volume of culture media (1:1 AIM-V/RPMI 1640+10% filtered human AB Serum+50 μM B-mercaptoethanol (TC grade)). Cells were rested overnight at 37 degrees Celsius in CO₂ incubator (5% CO₂).

After incubation, cells were treated with 50 ng mRNA encoding native CMV pp65 protein, 50 ng mRNA encoding pp65 with MHC presentation enhancing sequences, 2 ug/mL CMV pp65 peptide pool covering the entire pp65 molecule, or non-coding mRNA. Cells were incubated at 37 degrees Celsius in CO₂ incubator (5% CO₂) for 24 hrs.

After 24 hrs, cells and cell culture supernatant was harvested, and the cells were washed twice in phosphate buffered saline (PBS) pH 7.2. Washed cells were then stained with Zombie Near Infrared live dead stain (NIR)(BioLegend) in PBS for 15 minutes at room temperature (RT). Cells were then washed and re-suspended in 100 ul FACS buffer (PBS+0.5% BSA+0.02% Sodium Azid) containing fluorochrome conjugated a-CD8, a-CD4, a-CD137, and a-CD69 (BioLegend). Cells were then incubated for 20 minutes at room temperature.

After staining, cells were washed twice with 200 ul PBS followed by 10 minutes centrifugation at 1200 rpm. After the final wash, the supernatant was discarded, and cells were resuspended in 200 ul PBS. Resuspended cells were then analyzed on a flow cytometer (Cytek). The supernatant was used for measuring secreted Interferon gamma (IFNg) using standardized commercially available human IFNg ELISA kits and protocol (Thermo Scientific).

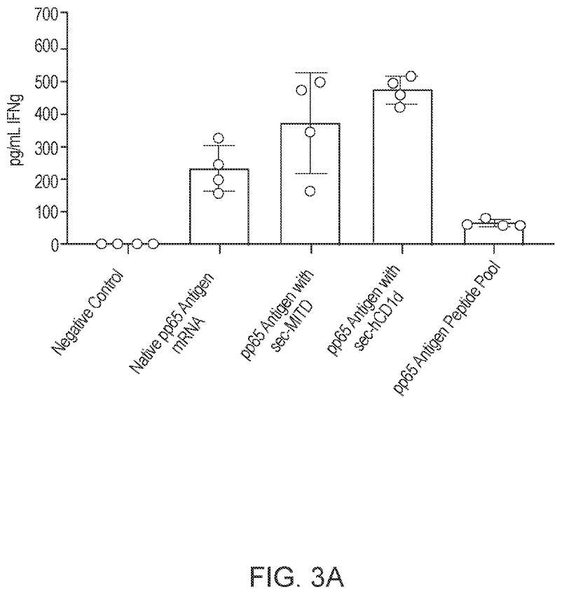

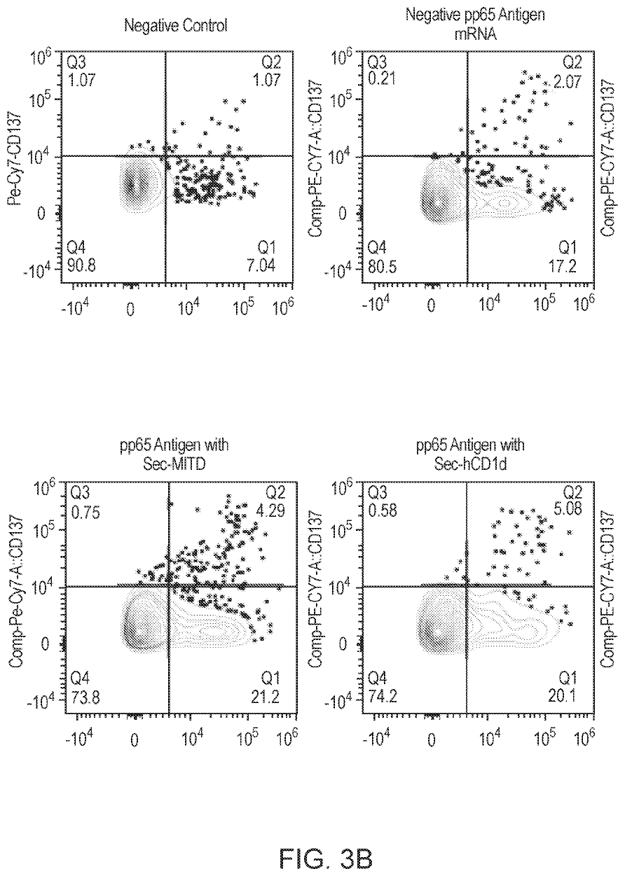

Results: As depicted in FIG. 3A, improved IFNg T cell responses were observed with Sec-hCD1d MHC-sorting sequences over peptides, native pp65 mRNA, and pp65 mRNA Sec-MITD. More activated CD8 T cells were observed in samples treated with Sec-hCD1d pp65 mRNA nanoparticles compared to native pp65 mRNA and pp65 mRNA Sec-MITD. (FIG. 3B). By introducing MHC presentation enhancing sequences, the antigen-presentation and CD8 T cell activation in these PBMC samples were improved.

### Example 3C

Cryopreserved Human Cytomegaly Virus (CMV) sero positive Healthy Donor (CMV+) Peripheral blood mononuclear cells were thawed and resuspended in 14 mL RPMI1640. Cells were pelleted by centrifugation at 1200 rpm for 10 minutes. The supernatant was aspired, and cells were re-suspended in and counted in appropriate volume of culture media (1:1 AIM-V/RPMI 1640+10% filtered human AB Serum+50 μM B-mercaptoethanol (TC grade)). Cells were rested overnight at 37 degrees Celsius in CO₂ incubator (5% CO₂).

After incubation 8×10⁶ cells were treated with either 1 μg mRNA encoding pp65 with Sec-hCD1d MHC presentation enhancing sequences, 2 μM CMV pp65 peptide pool covering the entire pp65 molecule, or non-coding mRNA. Cells were incubated at 37 degrees Celsius in CO₂ incubator (5% CO₂) in culture media without additional cytokines to support T cell growth for 6 days.

After 6 days, cells were harvested and CD8 T cells were isolated using a human CD8 isolation kit (STEMCELL). Viability was measured, cells were counted, and 50 000 isolated CD8 T cells from either peptide or mRNA treated samples were seeded in 8 replicates in 100 μl complete media in a 96-well U-bottom plate.

HLA-A2:01 expressing T2 cells (ATCC) cells were then labeled with cell tracer violet dye according to manufacturer protocol (Thermo Scientific). After labeling, cells were washed twice in pre-heated media before viability and cell number were assessed.

Half of the T2 cells were pulsed, while the other half was left un-pulsed, with CMV pp65 peptide at 37 degrees Celsius for 1 hr. After 1 hr, the cells were washed 2 times in pre-heated complete media before 10000 Pulsed or un-pulsed T2 cells were added to the wells containing the isolated CD8 T cells. Isolated CD8 T cells and pulsed or un-pulsed T2 cells were co-incubated for 4 hrs at 37 degrees Celsius. After 4 hrs, 50 propidium iodide (PI) was added to each well, and CD8 mediated T2 killing was analyzed by flow cytometry (Cytek).

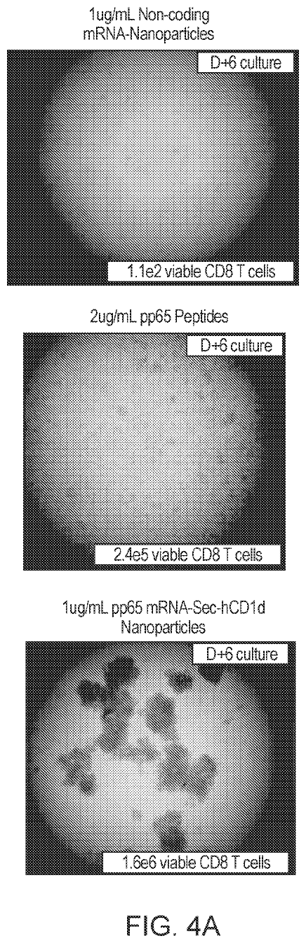

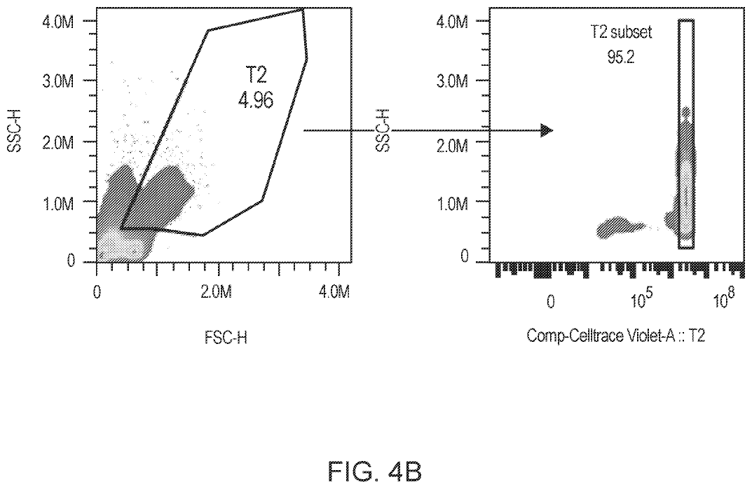

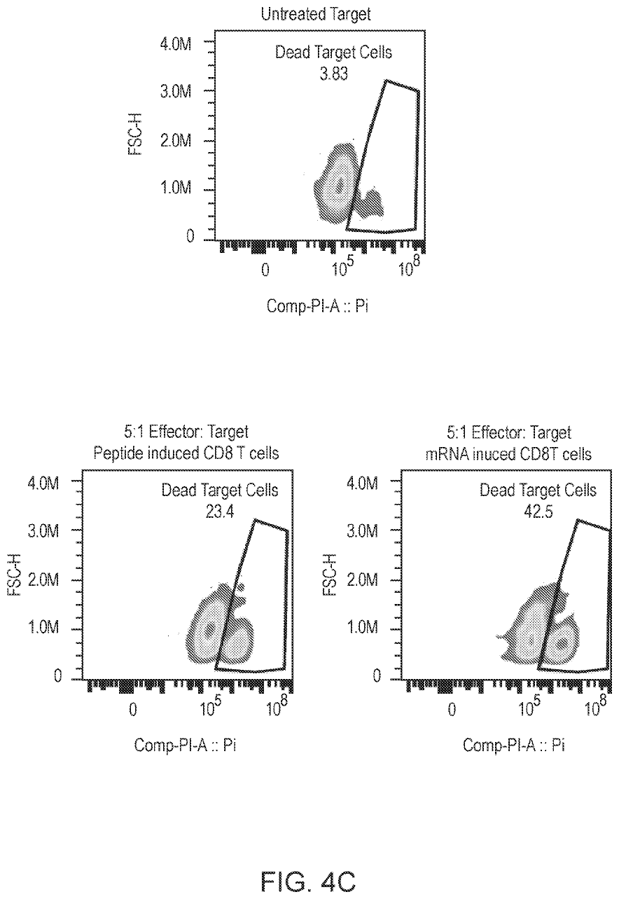

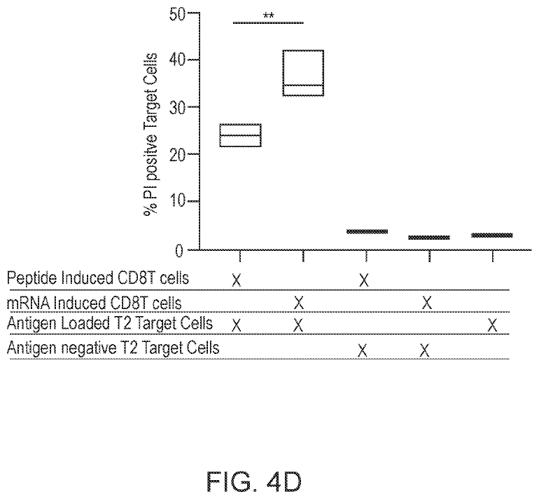

Results: After 6 days of passive expansion, robust CD8 T cell growth in cultures treated with 1 ug mRNA encoding pp65 with Sec-hCD1d was observed. Both viability and cell numbers were superior compared to cells treated with peptide. (FIG. 4A) The activated and expanded CD8 T cells were able to recognize and kill a T2 target cell only when the T2 target cell was pulsed with CMV-pp65 antigens. Significantly better killing efficacy in CD8 T cells isolated from cultures that were treated with mRNA compared to peptides was observed (FIGS. 4B-4D). This shows that large numbers of functional (i.e., able to kill target cells) CD8 T cells can be generated by treating whole PBMC populations with nanoparticles containing mRNAs encoding antigens and an MHC trafficking signal Sec-hCD1d.

### Example 3D

Cryopreserved Human Cytomegaly Virus (CMV) sero positive Healthy Donor (CMV+) Peripheral blood mononuclear cells were thawed and resuspended in 14 mL RPMI1640. Cells were pelleted by centrifugation at 1200 rpm for 10 minutes. The supernatant was aspired and cells were re-suspended in and counted in an appropriate volume of culture media (1:1 AIM-V/RPMI 1640+10% filtered human AB Serum+50 μM B-mercaptoethanol (TC grade)). Cells were rested overnight at 37 degrees Celsius in CO₂ incubator (5% CO₂).

After incubation 8×10⁶ cells were treated with either 1 μg mRNA encoding pp65 with Sec-hCD1d MHC presentation enhancing sequences (in duplicate), 2 uM CMV pp65 peptide pool covering the entire pp65 molecule, or non-coding mRNA. Cells were incubated at 37 degrees Celsius in CO₂ incubator (5% CO₂) in culture media for 24 hr. After 24 hrs, cells and cell culture supernatant was harvested, and cells were washed twice in phosphate buffered saline (PBS) pH 7.2. Washed cells were then stained with Zombie Near Infrared live dead stain (NIR)(BioLegend) in PBS for 15 minutes at room temperature (RT). Cells were then washed and re-suspended in 100 ul FACS buffer (PBS+0.5% BSA+0.02% Sodium Azid) containing fluorochrome conjugated a-CD8, a-CD4, a-CD137, and a-CD69 (BioLegend). Cells were then incubated for 20 minutes at room temperature.

After staining, cells were washed twice with 200 ul PBS followed by 10 minutes centrifugation at 1200 rpm. After the final wash, the supernatant was discarded, and cells were resuspended in 200 ul PBS. Resuspended cells were then single cell sorted based on the expression of CD137 and CD69 on a flow sorter (Aria BD biosciences). CD137 and CD69 double positive cells were sorted in Takara 10× buffer (Takara biosciences). Sorted cells were submitted to MedGenome (Medgenome) for T cell receptor sequencing.

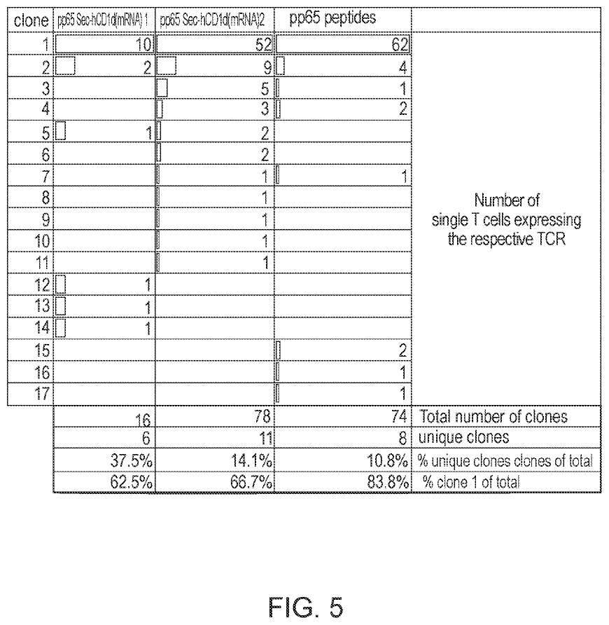

As depicted in FIG. 5, a higher clonal diversity among CD8 T cells sorted from pp65 Sec-hCD1d mRNA nanoparticle treated PBMCs compared to pp65 peptides treated was observed.

### Example 4. HPV16 E7 Protein Expression in HEK293 (Sec mRNA402 vs hCD1d mRNA416)

A culture of 50K HEK293 cells was treated overnight with 50 ng mRNA. The supernatant and cell lysate (freeze and thaw×3) was collected after 24 hr. transfection. The HPV17 E7 protein was measured by ELISA, using HPV16/18 E7 ELISA kits (CellBioLabs) or in-House method by direct sample coating on plates.

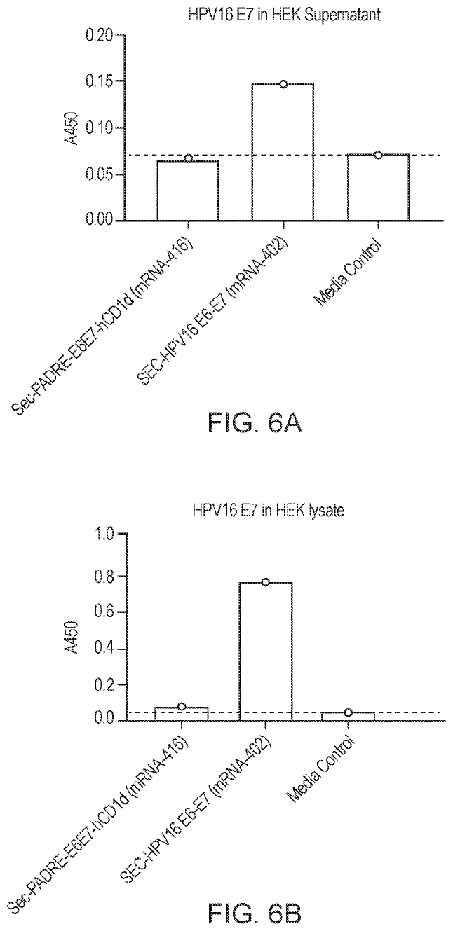

Results. As depicted in FIGS. 6A and 6B, transfection of HEK293 with SEC-HPV E6-E7 (mRNA 402) generated robust E7 protein comparing to the hCD1d mRNA −416.

### Equivalents and Scope

Those skilled in the art will recognize or be able to ascertain using no more than routine experimentation, many equivalents to the specific aspects in accordance with the disclosure described herein. The scope of the present disclosure is not intended to be limited to the above Description, but rather is as set forth in the appended claims.

In the claims, articles such as “a,” “an,” and “the” may mean one or more than one unless indicated to the contrary or otherwise evident from the context. Claims or descriptions that include “or” between one or more members of a group are considered satisfied if one, more than one, or all of the group members are present in, employed in, or otherwise relevant to a given product or process unless indicated to the contrary or otherwise evident from the context. The disclosure includes aspects in which exactly one member of the group is present in, employed in, or otherwise relevant to a given product or process. The disclosure includes aspects in which more than one, or the entire group members are present in, employed in, or otherwise relevant to a given product or process.

It is also noted that the term “comprising” is intended to be open and permits but does not require the inclusion of additional elements or steps. When the term “comprising” is used herein, the term “consisting of” is thus also encompassed and disclosed.

Where ranges are given, endpoints are included. Furthermore, it is to be understood that unless otherwise indicated or otherwise evident from the context and understanding of one of ordinary skill in the art, values that are expressed as ranges can assume any specific value or subrange within the stated ranges in different aspects of the disclosure, to the tenth of the unit of the lower limit of the range, unless the context clearly dictates otherwise.

In addition, it is to be understood that any particular aspect of the present disclosure that falls within the prior art may be explicitly excluded from any one or more of the claims. Since such aspects are deemed to be known to one of ordinary skill in the art, they may be excluded even if the exclusion is not set forth explicitly herein. Any particular aspect of the compositions of the disclosure (e.g., any antibiotic, therapeutic or active ingredient; any method of production; any method of use; etc.) can be excluded from any one or more claims, for any reason, whether or not related to the existence of prior art.

It is to be understood that the words which have been used are words of description rather than limitation and that changes may be made within the purview of the appended claims without departing from the true scope and spirit of the disclosure in its broader aspects.

While the present disclosure has been described at some length and with some particularity with respect to the several described aspects, it is not intended that it should be limited to any such particulars or aspects or any particular aspect, but it is to be construed with references to the appended claims so as to provide the broadest possible interpretation of such claims in view of the prior art and, therefore, to effectively encompass the intended scope of the disclosure.

Furthermore, “and/or” where used herein is to be taken as specific disclosure of each of the two specified features or components with or without the other. Thus, the term “and/or” as used in a phrase such as “A and/or B” herein is intended to include “A and B,” “A or B,” “A” (alone), and “B” (alone). Likewise, the term “and/or” as used in a phrase such as “A, B, and/or C” is intended to encompass each of the following aspects: A, B, and C; A, B, or C; A or C; A or B; B or C; A and C; A and B; B and C; A (alone); B (alone); and C (alone).

Unless defined otherwise, all technical and scientific terms used herein have the same meaning as commonly understood by one of ordinary skill in the art to which this disclosure is related. For example, the Concise Dictionary of Biomedicine and Molecular Biology, Juo, Pei-Show, 2nd ed., 2002, CRC Press; The Dictionary of Cell and Molecular Biology, 3rd ed., 1999, Academic Press; and the Oxford Dictionary Of Biochemistry And Molecular Biology, Revised, 2000, Oxford University Press, provide one of skill with a general dictionary of many of the terms used in this disclosure.

Wherever aspects are described herein with the language “comprising,” otherwise analogous aspects described in terms of “consisting of” and/or “consisting essentially of” are also provided.

Units, prefixes, and symbols are denoted in their Systeme International de Unites (SI) accepted form. Numeric ranges are inclusive of the numbers defining the range. Where a range of values is recited, it is to be understood that each intervening integer value, and each fraction thereof, between the recited upper and lower limits of that range is also specifically disclosed, along with each subrange between such values. The upper and lower limits of any range can independently be included in or excluded from the range, and each range where either, neither, or both limits are included is also encompassed within the disclosure. Where a value is explicitly recited, it is to be understood that values which are about the same quantity or amount as the recited value are also within the scope of the disclosure. Where a combination is disclosed, each subcombination of the elements of that combination is also specifically disclosed and is within the scope of the disclosure. Conversely, where different elements or groups of elements are individually disclosed, combinations thereof are also disclosed. Where any element of a disclosure is disclosed as having a plurality of alternatives, examples of that disclosure in which each alternative is excluded singly or in any combination with the other alternatives are also hereby disclosed; more than one element of a disclosure can have such exclusions, and all combinations of elements having such exclusions are hereby disclosed.

1-11. (canceled)

12. A polynucleotide having the formula:
Signal/Leader-payload-PRM
wherein
the Signal/Leader encodes a signal sequence, a leader sequence, or a sorting sequence, in frame with and upstream of a payload;
the payload is selected from the group consisting of an antigenic payload region, a detectable agent, and a therapeutic agent; and
the PRM encodes all or a portion of at least one parental receptor molecule region from one or more proteins or isoforms selected from the group consisting of CD1d, CD1e, LDLR, LDLRP, and LRP1 proteins.

13. The polynucleotide of claim 12, wherein the parental receptor molecule region is selected from the group consisting of an extracellular region, a transmembrane region, and a cytoplasmic region.

14. A host cell comprising the polynucleotide of claim 12.

15. A pharmaceutical composition comprising a polynucleotide of claim 12.

16. The pharmaceutical composition of claim 15, wherein the pharmaceutical composition is in the form of a vaccine.

17. The pharmaceutical composition of claim 15, further comprising one or more pharmaceutically acceptable excipients or one or more additional pharmaceutically active ingredients.

18. The pharmaceutical composition of claim 17, wherein the pharmaceutically acceptable excipients are selected from the group consisting of antiadherents, antioxidants, binders, coatings, compression aids, disintegrants, dyes, emollients, emulsifiers, fillers, film formers or coatings, flavors, fragrances, glidants, lubricants, preservatives, printing inks, sorbents, suspending or dispersing agents, sweeteners, and waters of hydration.

19. A therapeutic polynucleotide comprising a polynucleotide of claim 12 formulated with a delivery vehicle.

20. The therapeutic polynucleotide of claim 19, wherein the polynucleotide is encapsulated with the delivery vehicle.

21. The therapeutic polynucleotide of claim 19, wherein the delivery vehicle is selected from the group consisting of amphipathic molecules, amino-lipidated peptides, and tertiary amino lipidated cationic peptides.

22. A therapeutic composition comprising the therapeutic polynucleotide of claim 19.

23. The therapeutic composition of claim 22, wherein the therapeutic composition is in the form of a vaccine.

24. The therapeutic composition of claim 22, further comprising one or more therapeutically acceptable excipients or one or more additional therapeutically active ingredients.

25. The therapeutic composition of claim 24, wherein the therapeutically acceptable excipients are selected from the group consisting of antiadherents, antioxidants, binders, coatings, compression aids, disintegrants, dyes, emollients, emulsifiers, fillers, film formers or coatings, flavors, fragrances, glidants, lubricants, preservatives, printing inks, sorbents, suspending or dispersing agents, sweeteners, and waters of hydration.

26. The therapeutic composition of claim 22, wherein a therapeutically effective dose, prophylactically effective dose, or appropriate imaging dose of the or therapeutic composition is administered to a subject in need thereof.

27-29. (canceled)

30. The polynucleotide of claim 12, wherein the polynucleotide is to perform one of the following:
a) enable antigen processing and presentation;
b) traffic protein to the antigen presentation pathway;
c) improve T cell activation;
d) increase clonal diversity; or
e) any combination thereof.

31. The polynucleotide of claim 12, wherein the PRM encodes all or a portion of a transmembrane region from one or more proteins or isoforms selected from the group consisting of CD1d, CD1e, LDLR, LDLRP, and LRP1 proteins and all or a portion of a cytoplasmic region from one or more proteins or isoforms selected from the group consisting of CD1d, CD1e, LDLR, LDLRP, and LRP1 proteins.

32. The polynucleotide of claim 12, wherein the payload is an antigenic payload region having the formula (An1)n-Xo-(An2)p comprising:
(a) a first encoded antigenic payload (An1), wherein n is an integer from 1 to 10
(b) an encoded linker region (X), wherein o is an integer from 0 to 10, and
(c) a second encoded antigenic payload (An2), wherein p is an integer from 0 to 10.

33. The polynucleotide of claim 32, wherein the first encoded or second encoded antigenic payload encodes all or a portion of a tumor antigen or an infectious agent antigen.

34. The polynucleotide of claim 12, wherein the payload is a detectable agent selected from the group consisting of organic small molecules, inorganic compounds, nanoparticles, enzymes or enzyme substrates, fluorescent materials, luminescent materials, bioluminescent materials, chemiluminescent materials, radioactive materials, contrast agents, gadolinium, iron oxides, monocrystalline iron oxide nanoparticles, ultrasmall superparamagnetic iron oxide, manganese chelates, barium sulfate, iodinated contrast media, microbubbles, and perfluorocarbons.

35. The therapeutic composition of claim 26, wherein administration of the therapeutic composition results in the generation of functional T cells in the subject.

36. The therapeutic composition of claim 26, wherein administration of the therapeutic composition results in improved IFNg T cell responses in the subject.

37. The therapeutic composition of claim 26, wherein administration of the therapeutic composition results increased antigen processing and presentation in the subject.


SEQUENCE LISTING (26 sequences)

SEQ ID NO: 1 - protein, 8 aa, HOMO SAPIENSs
SIINFEKL

SEQ ID NO: 2 - protein, 21 aa, HOMO SAPIENS
MGLIALAVLACLLFLLIVGFT

SEQ ID NO: 3 - protein, 13 aa, HOMO SAPIENS
SRFKRQTSYQGVL

SEQ ID NO: 4 - protein, 19 aa, HOMO SAPIENS
MGCLLFLLLWALLQAWGSA

SEQ ID NO: 5 - protein, 16 aa, HOMO SAPIENS
MLFLLLPLLAVLPGDG

SEQ ID NO: 6 - protein, 17 aa, HOMO SAPIENS
MLLLPFQLLAVLFPGGN

SEQ ID NO: 7 - protein, 17 aa, HOMO SAPIENS
MLFLQFLLLALLLPGGD

SEQ ID NO: 8 - protein, 19 aa, HOMO SAPIENS
MLLLFLLFEGLCCPGENTA

SEQ ID NO: 9 - protein, 21 aa, HOMO SAPIENS
MGPWGWKLRWTVALLLAAAGT

SEQ ID NO: 10 - protein, 20 aa, HOMO SAPIENS
MLTPPLLLLLPLLSALVAAM

SEQ ID NO: 11 - protein, 21 aa, HOMO SAPIENS
GFIILAVIVPLLLLIGLALWF

SEQ ID NO: 12 - protein, 21 aa, HOMO SAPIENS
IVLAIIVPSLLLLLCLALWYM

SEQ ID NO: 13 - protein, 21 aa, HOMO SAPIENS
NWIALVVIVPLVILIVLVLWF

SEQ ID NO: 14 - protein, 21 aa, HOMO SAPIENS
SIFLILICLTVIVTLVILVVV

SEQ ID NO: 15 - protein, 22 aa, HOMO SAPIENS
ALSIVLPIVLLVFLCLGVFLLW

SEQ ID NO: 16 - protein, 25 aa, HOMO SAPIENS
HIASILIPLLLLLLLVLVAGVVFWY

SEQ ID NO: 17 - protein, 6 aa, HOMO SAPIENS
RKRCFC

SEQ ID NO: 18 - protein, 9 aa, HOMO SAPIENS
RRRSYQNIP

SEQ ID NO: 19 - protein, 10 aa, HOMO SAPIENS
KKHCSYQDIL

SEQ ID NO: 20 - protein, 63 aa, HOMO SAPIENS
DSRLKKQSSNKNILSPHTPSPVFLMGANTQDTKNSRHQFCLAQVSWIKNRVLKKWKTRLNQLW

SEQ ID NO: 21 - protein, 50 aa, HOMO SAPIENS
KNWRLKNINSINFDNPVYQKTTEDEVHICHNQDGYSYPSRQMVSLEDDVA

SEQ ID NO: 22 - protein, 100 aa, HOMO SAPIENS
KRRVQGAKGFQHQRMTNGAMNVEIGNPTYKMYEGGEPDDVGGLLDADFALDPDKPTNFTNPVYATLYMGGHGSRHSLASTDEKRELLGRGPEDEIGDPLA

SEQ ID NO: 23 - protein, 12 aa, HOMO SAPIENS
APQALQSYHLAA

SEQ ID NO: 24 - DNA, 710 bp, murine
ctagcgagagaaaagaagaguaagaagaaauauaagagccaccaugcgcuaccugccuuggcugcugcugugggcuuuucugcaaguguggggccagucugaggcccuggaauccaucaucaacuucgagaagcugaccgagcugaucguguucaucgugcugaucaugcugguggucgugggcgccgugguguacuacauuuggagaagaagaagcgccuaccaggacaucagaugaguuaauuaagcugccuucugcggggcuugccuucuggccaugcccuucuucucucccuugcaccuguaccucuuggucuuugaauaaagccugaguaggaagcccgggcggattaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaa

SEQ ID NO: 25 - DNA, 567 bp, HOMO SAPIENS
ctagcgagagaaaagaagaguaagaagaaauauaagagccaccaugggcugccugcuguuucugcugcuuugggcucugcugcaggccuggggaucugcccuggaauccaucaucaacuucgagaagcugaccgagaugggccugaucgcucuggcuguucuggccugucugcuguuccuccugaucgugggcuucaccagcagauucaagagacagaccagcuaccagggcgugcucugaguuaauuaagcugccuucugcggggcuugccuucuggccaugcccuucuucucucccuugcaccuguaccucuuggucuuugaauaaagccugaguaggaagcccgggcggattaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaa

SEQ ID NO: 26 - DNA, 569 bp, HOMO SAPIENS
ctagcgagagaaaagaagaguaagaagaaauauaagagccaccaugcugcugcugcccuuccagcugcuggcuguucuuuuuccuggcggcaaccuggaauccaucaucaacuucgagaagcugaccgagaucgugcuggccaucaucgugccuucucugcugcuccugcugugucuggcccugugguacaugagaagaagaagcuaccagaacauccccugaguuaauuaagcugccuucugcggggcuugccuucuggccaugcccuucuucucucccuugcaccuguaccucuuggucuuugaauaaagccugaguaggaagcccgggcggattaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaa